SEQUENCE LISTING (202 sequences)

SEQ ID NO: 1 - protein, 118 aa, synthetic construct
EVQLVESGGGSVQAGESLRLSCAASGSIFSSNAMAWYRQAPGKQRDLVAVINSVGITKYADSVKGRFTISGDNAKNTVYLQMNSLKPEDTAVYYCTSDARRGWDTRYWGQGTQVTVSS

SEQ ID NO: 2 - protein, 118 aa, synthetic construct
EVQLVESGGGSVQPGGSLRLSCAASGSIFSSTAMAWYRQAPGKQRDLVAVISTVGITKYADSVKGRFTISRDNAKNTVYLQMNSLRPEDTALYYCTSDARRGWDTRYWGQGTLVTVSS

SEQ ID NO: 3 - protein, 118 aa, synthetic construct
DVQLVESGGGSVQPGGSLRLSCAASGSIFSSTAMAWYRQAPGKQRDLVAVISTVGITKYADSVKGRFTISRDNAKNTVYLQMNSLRPEDTALYYCTSDARRGWDTRYWGQGTLVTVSS

SEQ ID NO: 4 - protein, 118 aa, synthetic construct
EVQLVESGGGSVQAGESLRLSCAASGSIFSSNAMAWYRQAPGKRRDLVAAINSVGVTKYADSVKGRFTISRDNAKNTVYLQMNSLKPEDTAVYYCTSDPRRGWDTRYWGQGTQVTVSS

SEQ ID NO: 5 - protein, 118 aa, synthetic construct
EVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSS

SEQ ID NO: 6 - protein, 118 aa, synthetic construct
DVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSS

SEQ ID NO: 7 - protein, 118 aa, synthetic construct
DVQLVESGGGLVQPGGSLRLSCAASGSIFSSTAMAWYRQAPGKRRDLVAAISTVGVTKYADSVKGRFTISRDNSKNTVYLQMNSLRPEDTAVYYCTSDPRRGWDTRYWGQGTLVTVSS

SEQ ID NO: 8 - protein, 118 aa, synthetic construct
EVQLVESGGGLVQPGGSLRLSCAASGSIFSSTAMAWYRQAPGKRRDLVAAISTVGVTKYADSVKGRFTISRDNSKNTVYLQMNSLRPEDTAVYYCTSDPRRGWDTRYWGQGTLVTVSS

SEQ ID NO: 9 - protein, 25 aa, synthetic construct
EVQLVESGGGSVQPGGSLRLSCAAS

SEQ ID NO: 10 - protein, 10 aa, synthetic construct
GSIFSSTAMA

SEQ ID NO: 11 - protein, 14 aa, synthetic construct
WYRQAPGKQRDLVA

SEQ ID NO: 12 - protein, 9 aa, synthetic construct
VISTVGITK

SEQ ID NO: 13 - protein, 39 aa, synthetic construct
YADSVKGRFTISRDNAKNTVYLQMNSLRPEDTALYYCTS

SEQ ID NO: 14 - protein, 10 aa, synthetic construct
DARRGWDTRY

SEQ ID NO: 15 - protein, 11 aa, synthetic construct
WGQGTLVTVSS

SEQ ID NO: 16 - protein, 25 aa, synthetic construct
EVQLVESGGGSVQAGESLRLSCAAS

SEQ ID NO: 17 - protein, 10 aa, synthetic construct
GSIFSSNAMA

SEQ ID NO: 18 - protein, 14 aa, synthetic construct
WYRQAPGKRRDLVA

SEQ ID NO: 19 - protein, 9 aa, synthetic construct
AINTVGVTK

SEQ ID NO: 20 - protein, 9 aa, synthetic construct
VINSVGITK

SEQ ID NO: 21 - protein, 10 aa, synthetic construct
DPRRGWDTRY

SEQ ID NO: 22 - skipped

SEQ ID NO: 23 - protein, 30 aa, synthetic construct
EVQLVESGGGSVQPGGSLRLSCAASGSIFS

SEQ ID NO: 24 - protein, 5 aa, synthetic construct
STAMA

SEQ ID NO: 25 - protein, 39 aa, synthetic construct
YADSVKGRFTISGDNAKNTVYLQMNSLKPEDTAVYYCTS

SEQ ID NO: 26 - protein, 16 aa, synthetic construct
VISTVGITKYADSVKG

SEQ ID NO: 27 - protein, 32 aa, synthetic construct
RFTISRDNAKNTVYLQMNSLRPEDTALYYCTS

SEQ ID NO: 28 - protein, 11 aa, synthetic construct
WGQGTQVTVSS

SEQ ID NO: 29 - protein, 25 aa, synthetic construct
DVQLVESGGGSVQPGGSLRLSCAAS

SEQ ID NO: 30-32 - skipped (3 entries)

SEQ ID NO: 33 - protein, 16 aa, synthetic construct
AINTVGVTKYADSVKG

SEQ ID NO: 34-36 - skipped (3 entries)

SEQ ID NO: 37 - protein, 115 aa, synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 38 - protein, 115 aa, synthetic construct [sequence omitted]

SEQ ID NO: 39 - protein, 115 aa, synthetic construct [sequence omitted]

SEQ ID NO: 40 - protein, 116 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTATYYCTIGGSLSRSSQGTLVTVSSA

SEQ ID NO: 41 - protein, 116 aa, synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTATYYCTIGGSLSRSSQGTLVTVSSA

SEQ ID NO: 42 - protein, 115 aa, synthetic construct [sequence omitted]

SEQ ID NO: 43 - protein, 116 aa, synthetic construct
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVKVSSA

SEQ ID NO: 44 - protein, 115 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSS

SEQ ID NO: 45 - protein, 116 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSA

SEQ ID NO: 46 - protein, 117 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSAA

SEQ ID NO: 47 - protein, 118 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSAAA

SEQ ID NO: 48 - protein, 116 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSG

SEQ ID NO: 49 - protein, 117 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSGG

SEQ ID NO: 50 - protein, 118 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSGGG

SEQ ID NO: 51 - protein, 116 aa, synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 52 - protein, 25 aa, synthetic construct
EVQLVESGGGVVQPGGSLRLSCAAS

SEQ ID NO: 53 - protein, 10 aa, synthetic construct
GFTFRSFGMS

SEQ ID NO: 54 - protein, 14 aa, synthetic construct
WVRQAPGKGPEWVS

SEQ ID NO: 55 - protein, 10 aa, synthetic construct
SISGSGSDTL

SEQ ID NO: 56 - protein, 39 aa, synthetic construct
YADSVKGRFTISRDNSKNTLYLQMNSLRPEDTALYYCTI

SEQ ID NO: 57 - protein, 6 aa, synthetic construct
GGSLSR

SEQ ID NO: 58 - protein, 11 aa, synthetic construct
SSQGTLVTVSS

SEQ ID NO: 59 - protein, 30 aa, synthetic construct
EVQLVESGGGVVQPGGSLRLSCAASGFTFR

SEQ ID NO: 60 - protein, 5 aa, synthetic construct
SFGMS

SEQ ID NO: 61 - protein, 30 aa, synthetic construct
EVQLVESGGGLVQPGGSLRLSCAASGSIFS

SEQ ID NO: 62 - protein, 17 aa, synthetic construct
SISGSGSDTLYADSVKG

SEQ ID NO: 63 - protein, 32 aa, synthetic construct
RFTISRDNSKNTLYLQMNSLRPEDTALYYCTI

SEQ ID NO: 64 - protein, 16 aa, synthetic construct
AISTVGVTKYADSVKG

SEQ ID NO: 65 - protein, 32 aa, synthetic construct
RFTISRDNSKNTVYLQMNSLRPEDTAVYYCTS

SEQ ID NO: 66 - protein, 5 aa, synthetic construct
GGGGS

SEQ ID NO: 67 - protein, 7 aa, synthetic construct
SGGSGGS

SEQ ID NO: 68 - protein, 8 aa, synthetic construct
GGGGSGGS

SEQ ID NO: 69 - protein, 9 aa, synthetic construct
GGGGSGGGS

SEQ ID NO: 70 - protein, 10 aa, synthetic construct
GGGGSGGGGS

SEQ ID NO: 71 - protein, 15 aa, synthetic construct
GGGGSGGGGSGGGGS

SEQ ID NO: 72 - protein, 18 aa, synthetic construct
GGGGSGGGGSGGGGSGGS

SEQ ID NO: 73 - protein, 20 aa, synthetic construct
GGGGSGGGGSGGGGSGGGGS

SEQ ID NO: 74 - protein, 25 aa, synthetic construct
GGGGSGGGGSGGGGSGGGGSGGGGS

SEQ ID NO: 75 - protein, 30 aa, synthetic construct
GGGGSGGGGSGGGGSGGGGSGGGGSGGGGS

SEQ ID NO: 76 - protein, 35 aa, synthetic construct
GGGGSGGGGSGGGGSGGGGSGGGGSGGGGSGGGGS

SEQ ID NO: 77 - protein, 40 aa, synthetic construct
GGGGSGGGGSGGGGSGGGGSGGGGSGGGGSGGGGSGGGGS

SEQ ID NO: 78 - protein, 15 aa, synthetic construct
EPKSCDKTHTCPPCP

SEQ ID NO: 79 - protein, 24 aa, synthetic construct
GGGGSGGGSEPKSCDKTHTCPPCP

SEQ ID NO: 80 - protein, 12 aa, synthetic construct
EPKTPKPQPAAA

SEQ ID NO: 81 - protein, 62 aa, synthetic construct
ELKTPLGDTTHTCPRCPEPKSCDTPPPCPRCPEPKSCDTPPPCPRCPEPKSCDTPPPCPRCP

SEQ ID NO: 82 - protein, 370 aa, synthetic construct
DVQLVESGGGSVQPGGSLRLSCAASGSIFSSTAMAWYRQAPGKQRDLVAVISTVGITKYADSVKGRFTISRDNAKNTVYLQMNSLRPEDTALYYCTSDARRGWDTRYWGQGTLVTVSSGGGGSGGGSEVQLVESGGGSVQPGGSLRLSCAASGSIFSSTAMAWYRQAPGKQRDLVAVISTVGITKYADSVKGRFTISRDNAKNTVYLQMNSLRPEDTALYYCTSDARRGWDTRYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 83 - protein, 369 aa, synthetic construct
DVQLVESGGGLVQPGGSLRLSCAASGSIFSSTAMAWYRQAPGKRRDLVAAISTVGVTKYADSVKGRFTISRDNSKNTVYLQMNSLRPEDTAVYYCTSDPRRGWDTRYWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGSIFSSTAMAWYRQAPGKRRDLVAAISTVGVTKYADSVKGRFTISRDNSKNTVYLQMNSLRPEDTAVYYCTSDPRRGWDTRYWGQGTLVTVSS

SEQ ID NO: 84 - protein, 355 aa, Homo sapiens
MDQFPESVTENFEYDDLAEACYIGDIVVFGTVFLSIFYSVIFAIGLVGNLLVVFALTNSKKPKSVTDIYLLNLALSDLLFVATLPFWTHYLINEKGLHNAMCKFTTAFFFIGFFGSIFFITVISIDRYLAIVLAANSMNNRTVQHGVTISLGVWAAAILVAAPQFMFTKQKENECLGDYPEVLQEIWPVLRNVETNFLGFLLPLLIMSYCYFRIIQTLFSCKNHKKAKAIKLILLVVIVFFLFWTPYNVMIFLETLKLYDFFPSCDMRKDLRLALSVTETVAFSHCCLNPLIYAFAGEKFRRYLYHLYGKCLAVLCGRSVHVDFSSSESQRSRHGSVLSSNFTYHTSDGDALLLL

SEQ ID NO: 85 - protein, 355 aa, Macaca fascicularis
MDPFPESVTENFEYDDSAEACYIGDIVAFGTVFLSIFYSVVFAIGLVGNLLVVFALTNSKKPKSVTDIYLLNLALSDLLFVATLPFWTHYVINEEGLQNAMCKFTTAFFFIGFFGSIFFITIISIDRYLAIVLAANSMNNRTVQHGVTISLGVWAAAILVAAPQFMFTKQKENECLGDYPEVLQEIWPVLRNVEANFLGFLLPLLIMSYCYFRIIQTLFSCKNHKKAKAIKLILLVVVVFFLFWTPYNVMIFLETLKLYDFFPSCDMRRDLRLALSVTETVAFSHCCLNPLIYAFAGEKFRRYLYHLYGKCLAVLCGRSVHVDFSPSESQRSRQGSVLSSNFTYHTSDGDASLLL

SEQ ID NO: 86 - protein, 4 aa, synthetic construct
KERE

SEQ ID NO: 87 - protein, 4 aa, synthetic construct
KQRE

SEQ ID NO: 88 - protein, 4 aa, synthetic construct
GLEW

SEQ ID NO: 89 - protein, 5 aa, synthetic construct
KEREL

SEQ ID NO: 90 - protein, 5 aa, synthetic construct
KEREF

SEQ ID NO: 91 - protein, 5 aa, synthetic construct
KQREL

SEQ ID NO: 92 - protein, 5 aa, synthetic construct
KQREF

SEQ ID NO: 93 - protein, 5 aa, synthetic construct
KEREG

SEQ ID NO: 94 - protein, 5 aa, synthetic construct
KQREW

SEQ ID NO: 95 - protein, 5 aa, synthetic construct
KQREG

SEQ ID NO: 96 - protein, 4 aa, synthetic construct
TERE

SEQ ID NO: 97 - protein, 5 aa, synthetic construct
TEREL

SEQ ID NO: 98 - protein, 4 aa, synthetic construct
TQRE

SEQ ID NO: 99 - protein, 5 aa, synthetic construct
TQREL

SEQ ID NO: 100 - protein, 4 aa, synthetic construct
KECE

SEQ ID NO: 101 - protein, 5 aa, synthetic construct
KECEL

SEQ ID NO: 102 - protein, 5 aa, synthetic construct
KECER

SEQ ID NO: 103 - protein, 4 aa, synthetic construct
KQCE

SEQ ID NO: 104 - protein, 5 aa, synthetic construct
KQCEL

SEQ ID NO: 105 - protein, 4 aa, synthetic construct
RERE

SEQ ID NO: 106 - protein, 5 aa, synthetic construct
REREG

SEQ ID NO: 107 - protein, 4 aa, synthetic construct
RQRE

SEQ ID NO: 108 - protein, 5 aa, synthetic construct
RQREL

SEQ ID NO: 109 - protein, 5 aa, synthetic construct
RQREF

SEQ ID NO: 110 - protein, 5 aa, synthetic construct
RQREW

SEQ ID NO: 111 - protein, 4 aa, synthetic construct
QERE

SEQ ID NO: 112 - protein, 5 aa, synthetic construct
QEREG

SEQ ID NO: 113 - protein, 4 aa, synthetic construct
QQRE

SEQ ID NO: 114 - protein, 5 aa, synthetic construct
QQREW

SEQ ID NO: 115 - protein, 5 aa, synthetic construct
QQREL

SEQ ID NO: 116 - protein, 5 aa, synthetic construct
QQREF

SEQ ID NO: 117 - protein, 4 aa, synthetic construct
KGRE

SEQ ID NO: 118 - protein, 5 aa, synthetic construct
KGREG

SEQ ID NO: 119 - protein, 4 aa, synthetic construct
KDRE

SEQ ID NO: 120 - protein, 5 aa, synthetic construct
KDREV

SEQ ID NO: 121 - protein, 5 aa, synthetic construct
DECKL

SEQ ID NO: 122 - protein, 5 aa, synthetic construct
NVCEL

SEQ ID NO: 123 - protein, 4 aa, synthetic construct
GVEW

SEQ ID NO: 124 - protein, 4 aa, synthetic construct
EPEW

SEQ ID NO: 125 - protein, 4 aa, synthetic construct
GLER

SEQ ID NO: 126 - protein, 4 aa, synthetic construct
DQEW

SEQ ID NO: 127 - protein, 4 aa, synthetic construct
DLEW

SEQ ID NO: 128 - protein, 4 aa, synthetic construct
GIEW

SEQ ID NO: 129 - protein, 4 aa, synthetic construct
ELEW

SEQ ID NO: 130 - protein, 4 aa, synthetic construct
GPEW

SEQ ID NO: 131 - protein, 4 aa, synthetic construct
EWLP

SEQ ID NO: 132 - protein, 4 aa, synthetic construct
GPER

SEQ ID NO: 133 - protein, 4 aa, synthetic construct
GLER

SEQ ID NO: 134 - protein, 4 aa, synthetic construct
ELEW

SEQ ID NO: 135 - protein, 5 aa, synthetic construct
VTVSS

SEQ ID NO: 136 - protein, 15 aa, synthetic construct
GGGSSGGGSSGGGSS

SEQ ID NO: 137 - protein, 370 aa, synthetic construct
DVQLVESGGGLVQPGGSLRLSCAASGSIFSSTAMAWYRQAPGKRRDLVAAISTVGVTKYADSVKGRFTISRDNSKNTVYLQMNSLRPEDTAVYYCTSDPRRGWDTRYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGSEVQLVESGGGLVQPGGSLRLSCAASGSIFSSTAMAWYRQAPGKRRDLVAAISTVGVTKYADSVKGRFTISRDNSKNTVYLQMNSLRPEDTAVYYCTSDPRRGWDTRYWGQGTLVTVSSA

SEQ ID NO: 138 - protein, 10 aa, synthetic construct
  SITE 2: note = X1=P or A
DXRRGWDTRY

SEQ ID NO: 139 - protein, 9 aa, synthetic construct
AINSVGVTK

SEQ ID NO: 140 - protein, 39 aa, synthetic construct
YADSVKGRFTISRDNAKNTVYLQMNSLKPEDTAVYYCTS

SEQ ID NO: 141 - protein, 25 aa, synthetic construct
DVQLVESGGGLVQPGGSLRLSCAAS

SEQ ID NO: 142 - protein, 9 aa, synthetic construct
AISTVGVTK

SEQ ID NO: 143 - protein, 39 aa, synthetic construct
YADSVKGRFTISRDNSKNTVYLQMNSLRPEDTAVYYCTS

SEQ ID NO: 144 - protein, 25 aa, synthetic construct [sequence omitted]

SEQ ID NO: 145 - protein, 30 aa, synthetic construct
EVQLVESGGGSVQAGESLRLSCAASGSIFS

SEQ ID NO: 146 - protein, 5 aa, synthetic construct
SNAMA

SEQ ID NO: 147 - protein, 16 aa, synthetic construct
VINSVGITKYADSVKG

SEQ ID NO: 148 - protein, 32 aa, synthetic construct
RFTISGDNAKNTVYLQMNSLKPEDTAVYYCTS

SEQ ID NO: 149 - protein, 30 aa, synthetic construct
DVQLVESGGGSVQPGGSLRLSCAASGSIFS

SEQ ID NO: 150 - protein, 16 aa, synthetic construct
AINSVGVTKYADSVKG

SEQ ID NO: 151 - protein, 32 aa, synthetic construct
RFTISRDNAKNTVYLQMNSLKPEDTAVYYCTS

SEQ ID NO: 152 - protein, 30 aa, synthetic construct
DVQLVESGGGLVQPGGSLRLSCAASGSIFS

SEQ ID NO: 153 - protein, 370 aa, synthetic construct
DVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGSEVQLVESGGGSVQPGGSLRLSCAASGSIFSSTAMAWYRQAPGKQRDLVAVISTVGITKYADSVKGRFTISRDNAKNTVYLQMNSLRPEDTALYYCTSDARRGWDTRYWGQGTLVTVSSA

SEQ ID NO: 154 - protein, 370 aa, synthetic construct
DVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGSEVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 155 - protein, 370 aa, synthetic construct
DVQLVESGGGSVQPGGSLRLSCAASGSIFSSTAMAWYRQAPGKQRDLVAVISTVGITKYADSVKGRFTISRDNAKNTVYLQMNSLRPEDTALYYCTSDARRGWDTRYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGSEVQLVESGGGSVQPGGSLRLSCAASGSIFSSTAMAWYRQAPGKQRDLVAVISTVGITKYADSVKGRFTISRDNAKNTVYLQMNSLRPEDTALYYCTSDARRGWDTRYWGQGTLVTVSSA

SEQ ID NO: 156 - protein, 392 aa, synthetic construct
DVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSGGGGSEVQLVESGGGSVQPGGSLRLSCAASGSIFSSTAMAWYRQAPGKQRDLVAVISTVGITKYADSVKGRFTISRDNAKNTVYLQMNSLRPEDTALYYCTSDARRGWDTRYWGQGTLVTVSSA

SEQ ID NO: 157 - protein, 381 aa, synthetic construct
DVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSGGGGSEVQLVESGGGSVQPGGSLRLSCAASGSIFSSTAMAWYRQAPGKQRDLVAVISTVGITKYADSVKGRFTISRDNAKNTVYLQMNSLRPEDTALYYCTSDARRGWDTRYWGQGTLVTVSSA

SEQ ID NO: 158 - protein, 396 aa, synthetic construct
DVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSGGGGSGGGGSGGGGSGGGGSEVQLVESGGGSVQPGGSLRLSCAASGSIFSSTAMAWYRQAPGKQRDLVAVISTVGITKYADSVKGRFTISRDNAKNTVYLQMNSLRPEDTALYYCTSDARRGWDTRYWGQGTLVTVSSA

SEQ ID NO: 159 - protein, 381 aa, synthetic construct
DVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGSEVQLVESGGGSVQPGGSLRLSCAASGSIFSSTAMAWYRQAPGKQRDLVAVISTVGITKYADSVKGRFTISRDNAKNTVYLQMNSLRPEDTALYYCTSDARRGWDTRYWGQGTLVTVSSA

SEQ ID NO: 160 - protein, 407 aa, synthetic construct
DVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSGGGGSGGGGSGGGGSGGGGSEVQLVESGGGSVQPGGSLRLSCAASGSIFSSTAMAWYRQAPGKQRDLVAVISTVGITKYADSVKGRFTISRDNAKNTVYLQMNSLRPEDTALYYCTSDARRGWDTRYWGQGTLVTVSSA

SEQ ID NO: 161 - protein, 370 aa, synthetic construct; identical to SEQ ID NO: 153

SEQ ID NO: 162 - protein, 396 aa, synthetic construct
DVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGSEVQLVESGGGSVQPGGSLRLSCAASGSIFSSTAMAWYRQAPGKQRDLVAVISTVGITKYADSVKGRFTISRDNAKNTVYLQMNSLRPEDTALYYCTSDARRGWDTRYWGQGTLVTVSSA

SEQ ID NO: 163 - protein, 407 aa, synthetic construct
DVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSGGGGSEVQLVESGGGSVQPGGSLRLSCAASGSIFSSTAMAWYRQAPGKQRDLVAVISTVGITKYADSVKGRFTISRDNAKNTVYLQMNSLRPEDTALYYCTSDARRGWDTRYWGQGTLVTVSSA

SEQ ID NO: 164 - protein, 370 aa, synthetic construct; identical to SEQ ID NO: 155

SEQ ID NO: 165 - protein, 381 aa, synthetic construct
DVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSEVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 166 - protein, 407 aa, synthetic construct
DVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSEVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 167 - protein, 396 aa, synthetic construct
DVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSGGGGSGGGGSGGGGSEVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 168 - protein, 407 aa, synthetic construct
DVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSGGGGSGGGGSGGGGSEVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 169 - protein, 370 aa, synthetic construct
DVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGSEVQLVESGGGSVQPGGSLRLSCAASGSIFSSTAMAWYRQAPGKQRDLVAVISTVGITKYADSVKGRFTISRDNAKNTVYLQMNSLRPEDTALYYCTSDARRGWDTRYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 170 - protein, 370 aa, synthetic construct
DVQLVESGGGSVQPGGSLRLSCAASGSIFSSTAMAWYRQAPGKQRDLVAVISTVGITKYADSVKGRFTISRDNAKNTVYLQMNSLRPEDTALYYCTSDARRGWDTRYWGQGTLVTVSSGGGGSGGGSEVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 171 - protein, 392 aa, synthetic construct
DVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSEVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 172 - protein, 422 aa, synthetic construct
DVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSGGGGSGGGGSGGGGSEVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 173 - protein, 381 aa, synthetic construct
DVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGSEVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 174 - protein, 396 aa, synthetic construct
DVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGSEVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 175 - protein, 422 aa, synthetic construct
DVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSGGGGSGGGGSGGGGSGGGGSEVQLVESGGGSVQPGGSLRLSCAASGSIFSSTAMAWYRQAPGKQRDLVAVISTVGITKYADSVKGRFTISRDNAKNTVYLQMNSLRPEDTALYYCTSDARRGWDTRYWGQGTLVTVSSA

SEQ ID NO: 176 - protein, 370 aa, synthetic construct
EVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGSEVQLVESGGGSVQPGGSLRLSCAASGSIFSSTAMAWYRQAPGKQRDLVAVISTVGITKYADSVKGRFTISRDNAKNTVYLQMNSLRPEDTALYYCTSDARRGWDTRYWGQGTLVTVSSA

SEQ ID NO: 177 - protein, 370 aa, synthetic construct
EVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGSEVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 178 - protein, 370 aa, synthetic construct
EVQLVESGGGSVQPGGSLRLSCAASGSIFSSTAMAWYRQAPGKQRDLVAVISTVGITKYADSVKGRFTISRDNAKNTVYLQMNSLRPEDTALYYCTSDARRGWDTRYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGSEVQLVESGGGSVQPGGSLRLSCAASGSIFSSTAMAWYRQAPGKQRDLVAVISTVGITKYADSVKGRFTISRDNAKNTVYLQMNSLRPEDTALYYCTSDARRGWDTRYWGQGTLVTVSSA

SEQ ID NO: 179 - protein, 392 aa, synthetic construct
EVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSGGGGSEVQLVESGGGSVQPGGSLRLSCAASGSIFSSTAMAWYRQAPGKQRDLVAVISTVGITKYADSVKGRFTISRDNAKNTVYLQMNSLRPEDTALYYCTSDARRGWDTRYWGQGTLVTVSSA

SEQ ID NO: 180 - protein, 381 aa, synthetic construct
EVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSGGGGSEVQLVESGGGSVQPGGSLRLSCAASGSIFSSTAMAWYRQAPGKQRDLVAVISTVGITKYADSVKGRFTISRDNAKNTVYLQMNSLRPEDTALYYCTSDARRGWDTRYWGQGTLVTVSSA

SEQ ID NO: 181 - protein, 396 aa, synthetic construct
EVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSGGGGSGGGGSGGGGSGGGGSEVQLVESGGGSVQPGGSLRLSCAASGSIFSSTAMAWYRQAPGKQRDLVAVISTVGITKYADSVKGRFTISRDNAKNTVYLQMNSLRPEDTALYYCTSDARRGWDTRYWGQGTLVTVSSA

SEQ ID NO: 182 - protein, 381 aa, synthetic construct
EVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGSEVQLVESGGGSVQPGGSLRLSCAASGSIFSSTAMAWYRQAPGKQRDLVAVISTVGITKYADSVKGRFTISRDNAKNTVYLQMNSLRPEDTALYYCTSDARRGWDTRYWGQGTLVTVSSA

SEQ ID NO: 183 - protein, 407 aa, synthetic construct
EVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSGGGGSGGGGSGGGGSGGGGSEVQLVESGGGSVQPGGSLRLSCAASGSIFSSTAMAWYRQAPGKQRDLVAVISTVGITKYADSVKGRFTISRDNAKNTVYLQMNSLRPEDTALYYCTSDARRGWDTRYWGQGTLVTVSSA

SEQ ID NO: 184 - protein, 370 aa, synthetic construct; identical to SEQ ID NO: 176

SEQ ID NO: 185 - protein, 396 aa, synthetic construct
EVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGSEVQLVESGGGSVQPGGSLRLSCAASGSIFSSTAMAWYRQAPGKQRDLVAVISTVGITKYADSVKGRFTISRDNAKNTVYLQMNSLRPEDTALYYCTSDARRGWDTRYWGQGTLVTVSSA

SEQ ID NO: 186 - protein, 407 aa, synthetic construct
EVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSGGGGSEVQLVESGGGSVQPGGSLRLSCAASGSIFSSTAMAWYRQAPGKQRDLVAVISTVGITKYADSVKGRFTISRDNAKNTVYLQMNSLRPEDTALYYCTSDARRGWDTRYWGQGTLVTVSSA

SEQ ID NO: 187 - protein, 370 aa, synthetic construct; identical to SEQ ID NO: 178

SEQ ID NO: 188 - protein, 370 aa, synthetic construct
EVQLVESGGGSVQPGGSLRLSCAASGSIFSSTAMAWYRQAPGKQRDLVAVISTVGITKYADSVKGRFTISRDNAKNTVYLQMNSLRPEDTALYYCTSDARRGWDTRYWGQGTLVTVSSGGGGSGGGSEVQLVESGGGSVQPGGSLRLSCAASGSIFSSTAMAWYRQAPGKQRDLVAVISTVGITKYADSVKGRFTISRDNAKNTVYLQMNSLRPEDTALYYCTSDARRGWDTRYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 189 - protein, 381 aa, synthetic construct
EVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSEVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 190 - protein, 407 aa, synthetic construct
EVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSEVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 191 - protein, 396 aa, synthetic construct
EVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSGGGGSGGGGSGGGGSEVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 192 - protein, 407 aa, synthetic construct
EVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSGGGGSGGGGSGGGGSEVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 193 - protein, 370 aa, synthetic construct
EVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGSEVQLVESGGGSVQPGGSLRLSCAASGSIFSSTAMAWYRQAPGKQRDLVAVISTVGITKYADSVKGRFTISRDNAKNTVYLQMNSLRPEDTALYYCTSDARRGWDTRYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 194 - protein, 370 aa, synthetic construct
EVQLVESGGGSVQPGGSLRLSCAASGSIFSSTAMAWYRQAPGKQRDLVAVISTVGITKYADSVKGRFTISRDNAKNTVYLQMNSLRPEDTALYYCTSDARRGWDTRYWGQGTLVTVSSGGGGSGGGSEVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 195 - protein, 392 aa, synthetic construct
EVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSEVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 196 - protein, 422 aa, synthetic construct
EVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSGGGGSGGGGSGGGGSEVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 197 - protein, 381 aa, synthetic construct
EVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGSEVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 198 - protein, 396 aa, synthetic construct
EVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGSEVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 199 - protein, 422 aa, synthetic construct
EVQLVESGGGSVQPGGSLRLSCAASGSIFSSTAMAWYRQAPGKRRDLVAAINTVGVTKYADSVKGRFTISRDNAKNTVYLQMNSLRPEDTALYYCTSDPRRGWDTRYWGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSGGGGSGGGGSGGGGSGGGGSEVQLVESGGGSVQPGGSLRLSCAASGSIFSSTAMAWYRQAPGKQRDLVAVISTVGITKYADSVKGRFTISRDNAKNTVYLQMNSLRPEDTALYYCTSDARRGWDTRYWGQGTLVTVSSA

SEQ ID NO: 200 - protein, 370 aa, synthetic construct
EVQLVESGGGLVQPGGSLRLSCAASGSIFSSTAMAWYRQAPGKRRDLVAAISTVGVTKYADSVKGRFTISRDNSKNTVYLQMNSLRPEDTAVYYCTSDPRRGWDTRYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGSEVQLVESGGGLVQPGGSLRLSCAASGSIFSSTAMAWYRQAPGKRRDLVAAISTVGVTKYADSVKGRFTISRDNSKNTVYLQMNSLRPEDTAVYYCTSDPRRGWDTRYWGQGTLVTVSSA

SEQ ID NO: 201 - protein, 369 aa, synthetic construct
EVQLVESGGGLVQPGGSLRLSCAASGSIFSSTAMAWYRQAPGKRRDLVAAISTVGVTKYADSVKGRFTISRDNSKNTVYLQMNSLRPEDTAVYYCTSDPRRGWDTRYWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGSIFSSTAMAWYRQAPGKRRDLVAAISTVGVTKYADSVKGRFTISRDNSKNTVYLQMNSLRPEDTAVYYCTSDPRRGWDTRYWGQGTLVTVSS

SEQ ID NO: 202 - protein, 9 aa, synthetic construct
  SITE 1: note = X1 = A or V
  SITE 3: note = X3= N or S
  SITE 7: note = X7= I or V
XIXTVGXTK

(19) United States
(12) Patent Application Publication
(10) Pub. No.: US 2024/0117038 A1
(43) Pub. Date: Apr. 11, 2024

(54) CX3CR1-BINDING COMPOUNDS, METHODS AND USES THEREOF

(71) Applicants: Ablynx N.V., Zwijnaarde (BE); Sanofi, Paris (FR)

(72) Inventors: Judith Verhelst, Zwijnaarde (BE); Paolo Meoni, Zwijnaarde (BE); Eric Lorent, Zwijnaarde (BE)

(73) Assignees: Ablynx N.V., Zwijnaarde (BE); Sanofi, Paris (FR)

(21) Appl. No.: 18/353,981

(22) Filed: Jul. 18, 2023

(30) Foreign Application Priority Data
Jul. 18, 2022 (EP) 22306067.4

(51) Int. Cl. C07K 16/28; A61P 29/00; G01N 33/68

(52) U.S. Cl. CPC C07K 16/28; A61P 29/00; G01N 33/6872; C07K 2317/565; C07K 2317/567; C07K 2317/569

(57) ABSTRACT

The present technology relates to a polypeptide comprising at least three immunoglobulin single variable domains (ISVDs). In particular, the present technology relates to a polypeptide comprising an optimized ISVD binding to fractalkine receptor CX3CR1 and an optimized ISVD binding to albumin. The present technology also relates to a composition of a polypeptide; a polynucleotide encoding a polypeptide; a host or host cell comprising a polynucleotide; a method for producing a polypeptide or composition; and a polypeptide, polynucleotide or composition for use in prevention, therapy and diagnosis of a disease or disorder.

### 1. TECHNICAL FIELD

The present technology relates to a polypeptide comprising immunoglobulin single variable domains (ISVDs) binding to CX3CR1. In particular, the present technology relates to a polypeptide comprising an optimized ISVD binding to fractalkine receptor CX3CR1 and an optimized ISVD binding to albumin. The present technology also relates to a composition of a polypeptide; a polynucleotide encoding a polypeptide; a host or host cell comprising a polynucleotide; a method for producing a polypeptide or composition; and a polypeptide, polynucleotide or composition for use in prevention, therapy and diagnosis of a disease or disorder.

### 2. BACKGROUND ART

C-X3-C motif chemokine receptor 1 (CX3CR1), also known as the fractalkine receptor or G-protein coupled receptor 13 (GPR13), is a transmembrane protein and a receptor for the chemokine fractalkine (also known as CX3CL1). CX3CR1-CX3CL1 signalling exerts distinct functions in different tissue compartments, such as immune response, inflammation, cell adhesion and chemotaxis (see e.g., Imai et al. 1997, Cell 91: 521-30). CX3CR1-CX3CL1 signalling is involved in the pathogenesis of various clinical disease states or processes, such as atherosclerosis, glomerulonephritis, cardiac allograft rejection, and rheumatoid arthritis (see e.g., Umehara et al. 2004, Arterioscler. Thromb. Vasc. Biol. 24: 34-40).

In addition, polymorphisms in CX3CR1 have been shown to be of clinical significance. For example, functional CX3CR1 analysis showed that fractalkine binding is reduced among patients homozygous for CX3CR1 variant (V2491/T280M), a variant haplotype affecting two amino acids (isoleucine-249 and methionine-280). CX3CR1 variant (V2491/T280M) has been associated with interindividual differences in susceptibility to HIV infection (see e.g., Faure et al. 2000, Science 287: 2274-7), atherosclerotic diseases and stroke (see e.g., McDermott et al. 2001, Circ. Res. 89: 401-7; Moatti et al. 2001, Blood 97: 1925-8). While HIV-infected patients homozygous for CX3CR1 variant (V2491/T280M) progressed to AIDS more rapidly than those with other haplotypes (see e.g., Faure et al. 2000, Science 287: 2274-7), CX3CR1 variant (V2491/T280M) has also been shown to be associated with a lower risk of cardiovascular disease (see e.g., McDermott et al. 2001, Circ. Res. 89: 401-7; McDermott et al. 2003, J. Clin. Invest. 111: 1241; Ghilardi et al. 2004, Stroke 35: 1276).

Hence, modulation of CX3CR1's activity could provide promising therapies. For example, several independent mouse genetic studies have shown a beneficial effect of CX3CR1 deficiency on atherosclerosis (Combadiere et al. 2003, Circulation 107: 1009; Lesnik et al. 2003, J. Clin. Invest. 111: 333).

A small molecule modulator of CX3CR1 activity, E-6130, was developed by Eisai Co. (see e.g., Wakita et al. 2017, Mol. Pharmacol. 92: 502) and entered clinical trials in 2016. However, in 2018, Eisai announced the discontinuation of these clinical studies. KAND-145 and KAND-567 are other small molecule antagonists of CX3CR1, being developed by Kancera in different diseases.

Quetmolimab (also known as E 6011), a humanized, anti-fractalkine (CX3CL1) monoclonal antibody was developed by Eisai (Tanaka et al. 2021, Modern Rheumatol. 31: 783). Also for this biological, clinical development has been discontinued.

Anti-CX3CR1 VHHs are described in WO 2013/130381. On this basis, BI 655088 (designated as A041600087 in the present specification) was developed as a therapeutic compound for the treatment artherosclerosis (see https://adisinsight.springer.com/drugs/800044864; Low et al. 2020, Mabs, 12: 1709322). However, as of today no therapeutic biologicals binding CX3CR1 are available and/or being developed for treating diseases associated with a fractalkine receptor CX3CR1 or a single nucleotide polymorphism (SNP) variant of CX3CR1.

Disadvantages of available CX3CR1 modulators, e.g., anti-CX3CR1 antibodies, include their manufacturing process and storage (e.g., low efficiency of production, low product quality) as well as their therapeutic applications (e.g., low half-life of CX3CR1 modulators, high immunogenicity, off-target binding).

Accordingly, there is a need in the art for improved CX3CR1-binding compounds to modulate CX3CR1 activity to prevent, treat and/or diagnose diseases.

### 3. SUMMARY

The present inventors developed polypeptides targeting CX3CR1 and comprising a half-life extending moiety that exhibited increased potency and/or efficacy of modulating CX3CR1 activity as compared to available anti-CX3CR1 therapeutic compounds. The polypeptides could be effectively produced (e.g. high expression yield in microbial hosts; better recovery after purification) and exhibited improved (long-term) stability under storage conditions (e.g. as measured by turbidity or opalescence of concentrations of 100 mg/mL of polypeptide). Furthermore, the polypeptides were shown to have limited reactivity to pre-existing antibodies (i.e., antibodies present in the subject before the first treatment with the polypeptide) in the subject to be treated. In certain embodiments, such polypeptides exhibit a half-life in a subject in need thereof (e.g., to prevent, treat or diagnose a disease or disorder associated with a fractalkine receptor CX3CR1 or a single nucleotide polymorphism (SNP) variant of CX3CR1) that is long enough such that consecutive treatments can be conveniently spaced apart. Moreover, such polypeptides could be shown to have improved product quality (e.g., reduced amount of low molecular weight species and/or reduced amount of low molecular high species and/or aggregation) after production and/or further purification.

In some embodiments, the present technology provides a polypeptide comprising or consisting of at least two immunoglobulin single variable domains (ISVDs) that specifically bind to CX3CR1 and/or a single nucleotide polymorphism (SNP) variant of CX3CR1 and a half-life extending moiety that specifically binds to a serum protein such as albumin. In a further embodiment, the polypeptide comprises or consists of two ISVDs that specifically bind to CX3CR1 and/or a single nucleotide polymorphism (SNP) variant of CX3CR1 and one ISVD that specifically binds to albumin, wherein the three ISVDs are optionally linked directly or via a peptidic linker.

In another aspect, a polynucleotide capable of expressing the polypeptide of the present technology, a vector comprising the polynucleotide, and a composition comprising the polypeptide, the polynucleotide or the vector are provided. In some embodiments, the composition is a pharmaceutical composition.

Also provided is a host or host cell comprising the polynucleotide or comprising a vector that encodes the polypeptide according to the present technology. Also provided are methods for producing the polypeptide and its use as a medicament in the prevention, therapy and diagnosis of a disease or disorder (e.g., an inflammatory disease, an atherosclerotic diseases or stroke).

The present technology meets or addresses at least some of the above needs and aims at solving the above problems in the art by providing improved products and methods which are defined by the independent claims. Particular embodiments are set out in the respective dependent claims, as well as in the specific embodiments described below. The present technology provides:
Item 1. A polypeptide comprising or consisting of at least three immunoglobulin single variable domains (ISVDs), wherein each of the at least three ISVDs has the structure FR1-CDR1-FR2-CDR2-FR3-CDR3-FR4 in which FR1 to FR4 refer to framework regions and CDR1 to CDR3 refer to complementarity determining regions; and wherein the polypeptide comprises
(a) a first ISVD specifically binding to fractalkine receptor CX3CR1 and/or a single nucleotide polymorphism (SNP) variant of CX3CR1 and comprising (according to AbM numbering)
a CDR1 as shown in SEQ ID NO: 10;
a CDR2 as shown in SEQ ID NO: 12; and
a CDR3 as shown in SEQ ID NO: 14; and
(b) a second ISVD specifically binding to fractalkine receptor CX3CR1 and/or a single nucleotide polymorphism (SNP) variant of CX3CR1 and comprising (according to AbM numbering)
a CDR1 as shown in SEQ ID NO: 10;
a CDR2 as shown in SEQ ID NO: 12; and
a CDR3 as shown in SEQ ID NO: 14; and
(c) a third ISVD specifically binding to albumin and comprising (according to AbM numbering)
a CDR1 as shown in SEQ ID NO: 53 or having two or one amino acid difference(s) with SEQ ID NO: 53;
a CDR2 as shown in SEQ ID NO: 55 or having two or one amino acid difference(s) with SEQ ID NO: 55; and
a CDR3 as shown in SEQ ID NO: 57 or having two or one amino acid difference(s) with SEQ ID NO: 57; and
wherein (a), (b), and (c) are linked directly or via a peptidic linker.
Item 2. The polypeptide according to the previous item, wherein the C-terminal ISVD of the polypeptide carries a C-terminal extension of FR4 comprising 1, 2, 3, 4, 5, 6, 7, 8, 9 or 10 amino acids in length.
Item 3. The polypeptide according to any one of the previous items, wherein the order of (a), (b), and (c) indicates their relative position to each other within the polypeptide, considered from the N-terminus to the C-terminus of said polypeptide.
Item 4. The polypeptide according to any one of the previous items, wherein the ISVD in (a), (b) and (c) is selected from a VH domain and a VHH domain.
Item 5. The polypeptide according to the previous item, wherein the ISVD in (a), (b) and (c) is selected from a camelized VH domain, a humanized VHH domain, a domain antibody and a dAb.
Item 6. The polypeptide according to the previous item, wherein the ISVD in (a), (b) and (c) is a humanized VHH domain.
Item 7. The polypeptide according to any one of the previous items, wherein in (a) and/or in (b), the ISVD comprises one or more of an aspartic acid (D) at amino acid position 1 according to Kabat numbering, a proline (P) at amino acid position 14 according to Kabat numbering, a glycine (G) at amino acid position 16 according to Kabat numbering, an arginine (R) at amino acid position 83 according to Kabat numbering, a leucine (L) at amino acid position 89 according to Kabat numbering and a leucine (L) at amino acid position 108 according to Kabat numbering.
Item 8. The polypeptide according to any one of the previous items, wherein in (a) and/or in (b), the ISVD comprises one or more of a serine (S) at amino acid position 11 according to Kabat numbering, and a glutamine (Q) at position 44 according to Kabat numbering.
Item 9. The polypeptide according to any one of the previous items, wherein in (a) and/or in (b), the ISVD comprises one or more of a threonine (T) at amino acid positions 32 according to Kabat numbering, a serine (S) at amino acid position 52 according to Kabat numbering and a threonine (T) at amino acid position 53 according to Kabat numbering.
Item 10. The polypeptide according to any one of the previous items, wherein in (a) and/or in (b), the ISVD comprises one or more of a valine (V) at amino acid position 50 according to Kabat numbering, an isoleucine (I) at amino acid position 56 according to Kabat numbering, and an alanine (A) at amino acid position 96 according to Kabat numbering.
Item 11. The polypeptide according to any one of the previous items, wherein in (a) and/or (b) the framework regions FR1 to FR4 comprise or consist of a FR1 as shown in SEQ ID NO: 9;
a FR2 as shown in SEQ ID NO: 11;
a FR3 as shown in SEQ ID NO: 13; and a FR4 as shown in SEQ ID NO: 15.
Item 12. The polypeptide according to any one of the previous items, wherein in (a) and/or in (b), the ISVD comprises an amino acid sequence having a sequence identity of more than 90% identity or more than 95% identity with SEQ ID NO: 2, wherein for the purpose of determining the % sequence identity, the amino acid sequence of the CDR regions are disregarded.
Item 13. The polypeptide according to any one of the previous items, wherein in (a) and/or in (b), the ISVD comprises an amino acid sequence having a sequence identity of more than 90% identity or more than 95% identity with SEQ ID NO: 2.
Item 14. The polypeptide according to any one of the previous items, wherein in (a) the ISVD consists of an amino acid sequence as shown in SEQ ID NO: 3 or an amino acid that has at least 90% sequence identity with SEQ ID NO:3 and in (b) the ISVD consists of an amino acid sequence as shown in SEQ ID NO: 2 or an amino acid that has at least 90% sequence identity with SEQ ID NO: 2, wherein for the purpose of determining the % sequence identity, the amino acid sequence of the CDR regions are disregarded.
Item 15. The polypeptide according to any one of the previous items, wherein in (a) the ISVD consists of an amino acid sequence as shown in SEQ ID NO: 3 or an amino acid that has at least 90% sequence identity with SEQ ID NO:3 and in (b) the ISVD consists of an amino acid sequence as shown in SEQ ID NO: 2 or an amino acid that has at least 90% sequence identity with SEQ ID NO: 2.
Item 16. The polypeptide according to any one of the previous items, wherein in (a) the ISVD consists of an amino acid sequence as shown in SEQ ID NO: 3 or an amino acid sequence having two or one amino acid difference(s) with SEQ ID NO: 2 and in (b) the ISVD consists of an amino acid sequence as shown in SEQ ID NO: 2 or an amino acid sequence having two or one amino acid difference(s) with SEQ ID NO: 2.
Item 17. The polypeptide according to any one of the previous items, wherein in (a) and/or in (b), the ISVD comprises an amino acid sequence as shown in SEQ ID NO: 2 or SEQ ID NO: 3.
Item 18. The polypeptide according to any one of the previous items, wherein in (a) the ISVD consists of an amino acid sequence as shown in SEQ ID NO: 3 and in (b) the ISVD consists of an amino acid sequence as shown in SEQ ID NO: 2.
Item 19. The polypeptide according to any one of the previous items, wherein in (a) the ISVD consists of an amino acid sequence as shown in SEQ ID NO: 3 and in (b) the ISVD consists of an amino acid sequence as shown in SEQ ID NO: 2, and wherein the order of (a), (b), and (c) indicates their relative position to each other within the polypeptide, considered from the N-terminus to the C-terminus of said polypeptide.
Item 20. The polypeptide according to any one of the previous items, wherein in (c) the ISVD specifically binds to human serum albumin.
Item 21. The polypeptide according to any one of the previous items, wherein in (c) the framework regions FR1 to FR4 comprise a FR1 as shown in SEQ ID NO: 52;
a FR2 as shown in SEQ ID NO: 54;
a FR3 as shown in SEQ ID NO: 56; and a FR4 as shown in SEQ ID NO: 58.
Item 22. The polypeptide according to any one of the previous items, wherein in (c) the ISVD comprises or consists of an amino acid sequence as shown in SEQ ID NO: 37 or an amino acid sequence having a sequence identity of more than 90% identity or more than 95% identity with SEQ ID NO: 37, wherein for the purpose of determining the % sequence identity the amino acid sequence of the CDR regions are disregarded.
Item 23. The polypeptide according to any one of the previous items, wherein in (c) the ISVD comprises or consists of an amino acid sequence as shown in SEQ ID NO: 37 or an amino acid sequence having a sequence identity of more than 90% identity or more than 95% identity with SEQ ID NO: 37.
Item 24. The polypeptide according to any one of the previous items, wherein in (c) the ISVD comprises or consists of an amino acid sequence as shown in SEQ ID NO: 37.
Item 25. The polypeptide according to any one of the previous items, wherein in (a) and in (b) the ISVD consists of an amino acid sequence as shown in SEQ ID NO: 2 or an amino acid sequence having two or one amino acid difference(s) with SEQ ID NO: 2 and in (c) the ISVD consists of an amino acid sequence as shown in SEQ ID NO: 37.
Item 26. The polypeptide according to any one of the previous items, wherein in (a) the ISVD consists of an amino acid sequence as shown in SEQ ID NO: 3, in (b) the ISVD consists of an amino acid sequence as shown in SEQ ID NO: 2 and in (c) the ISVD consists of an amino acid sequence as shown in SEQ ID NO: 37.
Item 27. The polypeptide according to any one of the previous items, wherein the ISVD that is located at the C-terminus of the polypeptide carries the C-terminal extension on the C-terminus of FR4, wherein said C-terminus of FR4 is SEQ ID NO: 135 and the C-terminal extension of FR4 comprises 1, 2, 3, 4 or 5 amino acids.
Item 28. The polypeptide according to the previous item, wherein the C-terminal extension of FR4 comprises or consists of one, two or three alanine (Ala) residue(s).
Item 29. The polypeptide according to the previous item, wherein the C-terminal extension of FR4 is a single Ala residue.
Item 30. The polypeptide according to any one of the previous items, wherein the peptidic linker comprises 3 to 62 amino acids.
Item 31. The polypeptide according to the previous item, wherein the amino acids in the peptidic linker are selected from alanine (A), glycine (G) and serine (S).
Item 32. The polypeptide according to the previous item, wherein the peptidic linker is selected from a 9 GS linker, a 20 GS linker and a 35 GS linker.
Item 33. The polypeptide according to the previous item, wherein the peptidic linker comprises or consists of a 9 GS linker.
Item 34. The polypeptide according to any one of the previous items, wherein the polypeptide comprises two peptidic linkers, wherein each peptidic linker is located between two ISVDs within the polypeptide;
wherein the two peptidic linkers comprise or consist of an amino acid sequence as shown in SEQ ID NO: 69.
Item 35. The polypeptide according to any one of the previous items, wherein the polypeptide comprises an amino acid sequence having a sequence identity of more than 90% identity or more than 95% identity with SEQ ID NO: 82.
Item 36. The polypeptide according to any one of the previous items, wherein the polypeptide comprises an amino acid sequence having a sequence identity of more than 90% identity or more than 95% identity with SEQ ID NO: 82, wherein for the purpose of determining the sequence identity, the CDR regions are disregarded.
Item 37. The polypeptide according to the previous item, wherein the polypeptide comprises an amino acid sequence as shown in SEQ ID NO: 82.
Item 38. The polypeptide according to the previous item, wherein the polypeptide consists of the amino acid sequence as shown in SEQ ID NO: 82.
Item 39. Polypeptide that consists of the amino acid sequence as shown in SEQ ID NO: 82
Item 40. The polypeptide according to any one of the previous items, wherein the single nucleotide polymorphism (SNP) variant of CX3CR1 comprises at least one SNP selected from the group comprising or consisting of SNPs rs938203, rs2669849, rs1050592, rs3732379, and rs3732378.
Item 41. The polypeptide according to any one of the previous items, wherein in (a) the first ISVD and/or in (b) the second ISVD are characterized by reduced antigenicity as compared to an ISVD consisting of an amino acid sequence as shown in SEQ ID NO: 1;
optionally wherein the antigenicity is assessed by the binding on Human Leukocyte Antigen DR isotype (HLA-DR) alleles.
Item 42. The polypeptide according to any one of the previous items, wherein the polypeptide is characterized by an increased yield after expression in yeast Pichia pastoris as compared to a reference polypeptide consisting of an amino acid sequence as shown in SEQ ID NO: 83.
Item 43. The polypeptide according to any one of the previous items, wherein the polypeptide is characterized by at least one improved biophysical property as compared to a reference polypeptide consisting of an amino acid sequence as shown in SEQ ID NO: 83;
wherein the biophysical property is selected from
   i. improved stability after purification of the polypeptide, e.g., as measured by turbidity/opalescence of concentrations of 100 mg/mL of polypeptide;
   ii. reduced amount of low molecular weight species after expression of the polypeptide in a suitable host, e.g., as measured by pre-peaks on reverse phase chromatography (e.g. RP-UPLC), e.g., after expression of the polypeptide in Pichia pastoris;
   iii. reduced amount of low molecular weight species after purification of the polypeptide e.g., as measured by capillary gel electrophoresis (CGE);
   iv. reduced amount of high molecular weight species after expression of the polypeptide in a suitable host, e.g., as measured by size exclusion high performance liquid chromatography (SE-HPLC); e.g., after expression of the polypeptide in Pichia pastoris;
   v. reduced amount of high molecular weight species after purification of the polypeptide, e.g., as measured by size exclusion high performance liquid chromatography (SE-HPLC); and
   vi. combinations of two or more of said biophysical properties.
Item 44. The polypeptide according to any one of the previous items, wherein the polypeptide is characterized by at least one improved biophysical property as compared to a reference polypeptide consisting of an amino acid sequence as shown in SEQ ID NO: 83;
wherein the biophysical property is selected from
   i. improved stability after purification of the polypeptide, as measured by turbidity/opalescence of concentrations of 100 mg/mL of polypeptide;
   ii. reduced amount of low molecular weight species after expression of the polypeptide in Pichia pastoris, as measured by pre-peaks on reverse phase chromatography (e.g. RP-UPLC);
   iii. reduced amount of low molecular weight species after purification of the polypeptide as measured by capillary gel electrophoresis (CGE);
   iv. reduced amount of high molecular weight species after expression of the polypeptide in Pichia pastoris, as measured by size exclusion high performance liquid chromatography (SE-HPLC);
   v. reduced amount of high molecular weight species after purification of the polypeptide, as measured by size exclusion high performance liquid chromatography (SE-HPLC); and
   vi. combinations of two or more of said biophysical properties.
Item 45. The polypeptide according to any one of the previous items, wherein the polypeptide is characterized by a higher recovery after a purification step as compared to the recovery of a reference polypeptide consisting of an amino acid sequence as shown in SEQ ID NO: 83, wherein the purification step is selected from:
   i. a capture step on a resin;
   ii. a pH adjustment step after the capture step on a resin;
   iii. a polish step;
   iv. an ultrafiltration/diafiltration/ultrafiltration step; and
   v. combinations of two or more of said purification steps.
Item 46. The polypeptide according to the previous item, wherein the capture step (i) is a purification step on protein A chromatography.
Item 47. The polypeptide according to any one of the previous items, wherein the polish step (iii) is an ion exchange chromatography step.
Item 48. The polypeptide according to any one of the previous items, wherein the polypeptide has a reduced amount of missing disulfide bridges after expression of the polypeptide in a suitable host compared to a reference polypeptide consisting of an amino acid sequence as shown in SEQ ID NO: 83, wherein the amount of polypeptides with the reduced amount of missing disulfide bridges is measured by reverse phase high performance liquid chromatography (RP-HPLC).
Item 49. The polypeptide according to the previous item, wherein the host is Pichia.
Item 50. The polypeptide according to any one of the previous items, wherein the polypeptide binds cell surface glycoprotein MUC18 with a lower affinity compared to the binding by a reference polypeptide consisting of an amino acid sequence as shown in SEQ ID NO: 83.
Item 51. A polynucleotide encoding a polypeptide according to any one of the previous items.
Item 52. A (non-human) host or host cell comprising a polynucleotide according to the previous item.
Item 53. A composition comprising a polypeptide or polynucleotide according to any one of the previous items; optionally wherein the composition is a pharmaceutical composition; and further optionally wherein the composition comprises at least one pharmaceutically acceptable carrier, diluent or excipient and/or adjuvant.
Item 54. A method for producing a polypeptide or composition according to any one of the previous items comprising steps of:
   expressing the polypeptide in a suitable host cell or host organism or in another suitable expression system;
   and further optionally
   isolating and/or purifying the polypeptide.
Item 55. A polypeptide, polynucleotide or composition according to any one of the previous items for use in inhibiting the binding of CX3CR1 to fractalkine in a mammalian cell.
Item 56. A polypeptide, polynucleotide or composition according to any one of the previous items for use as a medicament.
Item 57. A polypeptide, polynucleotide or composition according to any one of the previous items for use in prevention, therapy and/or diagnosis of a disease or disorder.
Item 58. The polypeptide, polynucleotide or composition for use according to the previous item, wherein the disease or disorder is a disease or disorder associated with a fractalkine receptor CX3CR1 or a single nucleotide polymorphism (SNP) variant of CX3CR1.
Item 59. The polypeptide, polynucleotide or composition for use according to the previous item, wherein the disease or disorder is selected from inflammatory disease, cardio- and cerebrovascular atherosclerotic disorder, peripheral artery disease, myocardial infarction, restenosis, diabetic nephropathy, glomerulonephritis, human crescentic glomerulonephritis, IgA nephropathy, membranous nephropathy, lupus nephritis, vasculitis including Henoch-Schonlein purpura and Wegener's granulomatosis, rheumatoid arthritis, graft versus host disease, atopic dermatitis, inflammatory bowel disease, Crohn's disease, osteoarthritis, allograft rejection, systemic sclerosis, neurodegenerative disorder and demyelinating disease, multiple sclerosis (MS), Alzheimer's disease, pulmonary disease such as COPD, asthma, neuroinflammation, neuropathic pain, inflammatory pain, and cancer, including ovarian cancer.
Item 60. A method for inhibiting the binding of CX3CR1 to fractalkine in a mammalian cell, comprising administering to the cell a polypeptide according to any one of the previous items, whereby signalling mediated by the fractalkine is inhibited.
Item 61. A method of treating a disease or disorder, wherein the disease or disorder is selected from inflammatory disease, cardio- and cerebrovascular atherosclerotic disorder, peripheral artery disease, myocardial infarction, restenosis, diabetic nephropathy, glomerulonephritis, human crescentic glomerulonephritis, IgA nephropathy, membranous nephropathy, lupus nephritis, vasculitis including Henoch-Schonlein purpura and Wegener's granulomatosis, rheumatoid arthritis, graft versus host disease, atopic dermatitis, inflammatory bowel disease, Crohn's disease, osteoarthritis, allograft rejection, systemic sclerosis, neurodegenerative disorder and demyelinating disease, multiple sclerosis (MS), Alzheimer's disease, pulmonary disease such as COPD, asthma, neuroinflammation, neuropathic pain, inflammatory pain, and cancer, including ovarian cancer, and wherein said method comprises administering, to a subject in need thereof, a pharmaceutically active amount of the polypeptide, polynucleotide or composition according to any one of the previous items.
Item 62. Use of the polypeptide, polynucleotide or composition according to any one of the previous items in the preparation of a pharmaceutical composition for inhibiting the binding of CX3CR1 to fractalkine in a mammalian cell.
Item 63. Use of the polypeptide, polynucleotide or composition according to any one of the previous items in the preparation of a pharmaceutical composition for treating a disease or disorder.
Item 64. Use of the polypeptide, polynucleotide or composition according to any one of the previous items in the preparation of a pharmaceutical composition for treating a disease or disorder, wherein the disease or disorder is a disease or disorder associated with a fractalkine receptor CX3CR1 or a single nucleotide polymorphism (SNP) variant of CX3CR1.
Item 65. Use of the polypeptide, polynucleotide or composition according to any one of the previous items in the preparation of a pharmaceutical composition for treating a disease or disorder selected from inflammatory disease, cardio- and cerebrovascular atherosclerotic disorder, peripheral artery disease, myocardial infarction, restenosis, diabetic nephropathy, glomerulonephritis, human crescentic glomerulonephritis, IgA nephropathy, membranous nephropathy, lupus nephritis, vasculitis including Henoch-Schonlein purpura and Wegener's granulomatosis, rheumatoid arthritis, graft versus host disease, atopic dermatitis, inflammatory bowel disease, Crohn's disease, osteoarthritis, allograft rejection, systemic sclerosis, neurodegenerative disorder and demyelinating disease, multiple sclerosis (MS), Alzheimer's disease, pulmonary disease such as COPD, asthma, neuroinflammation, neuropathic pain, inflammatory pain, and cancer, including ovarian cancer.

### 4. DESCRIPTION OF THE DRAWINGS

FIG. 1. Alignment of sequences of the ISVD building block described in WO 2013/130381 (66B02, 54A12 and 307) and exemplary ISVD building blocks of the present technology (66B02_SO and 54A12_SO).

FIG. 2. Expression profiling of exemplary multivalent ISVD constructs as described in Example 4. The amount of expressed protein was quantified using ProtA biosensors on Octet RED384.

FIG. 3(A-B). Binding by exemplary multivalent ISVD constructs (A041600025, A041600034, A041600035, A041600041, A041600085, A041600087) to CHO K1 cells expressing (FIG. 3A) human CX3CR1 and (FIG. 3B) cyno CX3CR1.

FIG. 4(A-B). CX3CL1 competition assay of huCX3CL1-AF647 with exemplary multivalent ISVD constructs (A041600025, A041600034, A041600035, A041600041, A041600085) and negative control (IRR00163; VHH reference) on CHO K1 cells expressing (FIG. 4A) human CX3CR1 and (FIG. 4B) cyno CX3CR1.

FIG. 5. Inhibition of CX3CL1 induced chemotaxis by exemplary multivalent ISVD constructs (A041600025, A041600034, A041600035, A041600041, A041600085, A041600087), as measured by luminescence (RLU).

FIG. 6(A-D). Binding by exemplary multivalent ISVD constructs (A041600025, A041600034, A041600035, A041600041, A041600085) (see top panel) and human CX3CL1/Fractalkine (full length)-Alexa647 (see bottom panel) to HEK293T cells transfected with different human CX3CR1 variants: (FIG. 6A) human CX3CR1 wildtype (WT); (FIG. 6B) human CX3CR1 V2491; (FIG. 6C) human CX3CR1 T280M; (FIG. 6D) human CX3CR1 V2491 and T280M.

FIG. 7. Binding by exemplary multivalent ISVD constructs (A041600025, A041600034, A041600035, A041600041, A041600085, A041600087) (see top panel) and purified anti-human CCR2 (see bottom panel) to CHO K1 human CCR2.

FIG. 8. Binding by exemplary multivalent ISVD constructs (A041600025, A041600034, A041600035, A041600041, A041600085, A041600087) (see top panel) and purified mouse anti-human CCR5 (see bottom panel) to CHO K1 human CCR5.

FIG. 9. Level of high molecular weight products (HMWs) for exemplary multivalent ISVD constructs (A041600025, A041600034, A041600035, A041600041, A041600085) during large scale purification, after capture (Load Polish), after polish (Load UF/DF/UF), and in the formulated product (Formulation), as determined by size exclusion high performance liquid chromatography (SE-HPLC).

FIG. 10. Level of low molecular weight products (LMWs) for exemplary multivalent ISVD constructs (A041600025, A041600034, A041600035, A041600041, A041600085) during large scale purification, after capture (Load Polish), after polish (Load UF/DF/UF), and in the formulated product (Formulation), as determined by capillary gel electrophoresis (GCE).

FIG. 11. Visual inspection of exemplary multivalent ISVD constructs (top panel: A041600085, A041600034, A041600035; bottom panel: A041600025, A041600041) by direct observation of the final formulated product. For ISVD constructs with three flask batches: left flask, buffer; middle flask, low concentration batch (50 mg/mL); and right flask, high concentration batch (100 mg/mL). For ISVD constructs with two flask batches: left flask: low concentration batch (50 mg/mL); right flask: high concentration batch (100 mg/mL). At the high concentration of 100 mg/mL, four out of the five multivalent ISVD constructs were found ‘Slightly Turbid’, and significant turbidity and/or opalescence was observed for A041600025 and was therefore classified as ‘Turbid’.

FIG. 12. Turbidity measurement of exemplary multivalent ISVD constructs (A041600025, A041600034, A041600035, A041600041, A041600085) by OD500 measurement of the final formulated product.

FIG. 13. Characterization of exemplary multivalent ISVD constructs (A041600034, A041600035, A041600085) for blocking human CX3CL1 induced phosphorylation of ERK in BA/F3 cells expressing human CX3CR1, as measured by the ratio of pERK signal, as described in Example 8.1.

FIG. 14(A-D). Characterization of exemplary multivalent ISVD construct (A041600035) and anti-mouse CX3CR1 for inhibition of ERK phosphorylation induced by CX3CL1 in CHO K1 cells expressing CX3CR1, as measured by the ratio of pERK signal, as described in Example 8.1. FIG. 14A: ERK phosphorylation induced by human CX3CL1 in CHO K1 cells expressing human CX3CR1. FIG. 14B: ERK phosphorylation induced by mouse CX3CL1 in CHO K1 cells expressing human CX3CR1. FIG. 14C: ERK phosphorylation induced by human CX3CL1 in CHO K1 cells expressing mouse CX3CR1. FIG. 14D: ERK phosphorylation induced by mouse CX3CL1 in CHO K1 cells expressing mouse CX3CR1.

FIG. 15(A-C). Monocyte chemotaxis assay of exemplary multivalent ISVD constructs (A041600035, A014600087) and negative controls (neg. ctrl 1: IRR00164, ISVD which does not bind CX3CR1; neg. ctrl 2: no ISVD/antibody) in trans-well culture from healthy donors (n=3), as measured by arbitrary migration index (fold change to the media control wells) of (FIG. 15A) classical monocytes, (FIG. 15B) intermediate monocytes and (FIG. 15C) non-classical monocytes.

FIG. 16. Monocyte chemotaxis assay in trans-well culture from healthy donors (n=5; top panel) and Lupus Nephritis (LN) donors (n=6; bottom panel) as measured by arbitrary migration index of total monocytes (left), CD16+ monocytes (middle) and CD16− monocytes (right). *=p value <0.05; ***=p value <0.001; ns=p value >0.05.

FIG. 17(A-B). Binding of exemplary multivalent ISVD constructs (FIG. 17A) A041600035 and (FIG. 17B) A041600087 was evaluated in a human membrane protein array (MPA) to profile specificity and target selectivity. Binding across the protein library (including vector, DCC, DSC1, MCAM, FCGR2B, CX3CR1) and a positive control (Protein A) were measured on an Intellicyt iQue using the fluorescently labeled secondary antibody on unfixed cells.

FIG. 18. Pharmacokinetic (PK) profile of exemplary multivalent ISVD constructs A041600035 and A041600087 in Cynomolgus monkey for a normalized IV single-dose of 0.1 mg/kg and 1 mg/kg.

FIG. 19. Design of 7-day non-accelerated NTN mouse model.

FIG. 20(A-C). Representative illustrations of the alteration of F4/80 (FIG. 20A), CD11b/CD4 (FIG. 20B) and Mac-2/Galectin-3 (FIG. 20C) distribution patterns over renal cortex of one hCX3CR1 KI mice treated with IRR00163 in comparison to one naive mouse at day 7 after NTS unique injection.

FIG. 21. Design of 21-day non-accelerated NTN mouse model.

FIG. 22(A-C). Representative illustrations of the alteration of F4/80 (FIG. 22A), CD11b-CD4 (FIG. 22B) and Mac-2/Galectin-3-CD8 (FIG. 22C) distribution patterns over renal cortex of hCX3CR1 KI mice treated with NTS and IRR00163 in comparison to naïve mouse at day 21 after NTS unique injection.

### 5. DETAILED DESCRIPTION

### 5.1 Definitions

All publications, patents and patent applications cited herein are hereby incorporated by reference in their entirety for all purposes.

In accordance with the present technology, each occurrence of the term “comprising” may optionally be substituted with the term “consisting of”. The articles “a/an” and “the” are used herein to refer to one or to more than one (e.g., to at least one) of the grammatical object of the article unless otherwise clearly indicated by contrast. By way of example, “an element” means one element or more than one element. The term “or” is used herein to mean, and is used interchangeably with, the term “and/or”, unless context clearly indicates otherwise.

The term “about” as used herein refers to a deviation of ±10% from the recited value. When the term “about” is used herein in reference to a number, it should be understood that still another embodiment includes that number not modified by the presence of the term “about”. In the absence of the term “about” and unless the context dictates otherwise, generally accepted rounding rules apply to the specified values.

Unless indicated or defined otherwise, all terms used have their usual meaning in the art, which will be clear to the skilled person. Reference is for example made to the standard handbooks, such as Sambrook et al. 1989 (Molecular Cloning: A Laboratory Manual, 2nd Ed., Vols. 1-3, Cold Spring Harbor Laboratory Press), Ausubel et al. 1987 (Current protocols in molecular biology, Green Publishing and Wiley Interscience, New York), Lewin 1985 (Genes II, John Wiley & Sons, New York, N.Y.), Old et al. 1981 (Principles of Gene Manipulation: An Introduction to Genetic Engineering, 2nd Ed., University of California Press, Berkeley, CA), Roitt et al. 2001 (Immunology, 6th Ed., Mosby/Elsevier, Edinburgh), Roitt et al. 2001 (Roitt's Essential Immunology, 10th Ed., Blackwell Publishing, UK), and Janeway et al. 2005 (Immunobiology, 6th Ed., Garland Science Publishing/Churchill Livingstone, New York), as well as to the general background art cited herein.

Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein; as well as to for example the following reviews: Presta 2006 (Adv. Drug Deliv. Rev. 58: 640), Levin and Weiss 2006 (Mol. Biosyst. 2: 49), Irving et al. 2001 (J. Immunol. Methods 248: 31), Schmitz et al. 2000 (Placenta 21 Suppl. A: S106), Gonzales et al. 2005 (Tumour Biol. 26: 31), which describe techniques for protein engineering, such as affinity maturation and other techniques for improving the specificity and other desired properties of proteins such as immunoglobulins.

The terms “polypeptide”, “protein”, or “peptide” are used and each refers to a linear polymer composed of covalently linked amino acids (also referred to as “amino acid sequence”) which may be composed of natural L-amino acids (commonly found in naturally occurring proteins). Any amino acid sequence that comprises post-translationally modified amino acids (e.g. methylation, phosphorylation, actylation, amidation, hydroxylations, formylation or glycosylations) may be described as the amino acid sequence that is initially translated, i.e. these modifications shall not be shown explicitly in the amino acid sequence. Any polypeptide that can be expressed as a sequence modified linkages, cross links and end caps, non-peptidyl bonds, etc., is embraced by this definition. In some instances, a polypeptide may comprise a N- and/or C-terminal protecting group. In some instances, a polypeptide may comprise one or more non-natural amino acid(s).

TABLE 1
List of 20 proteinogenic amino acids.

| Amino acid | Three letter code | One letter code |
|---|---|---|
| Alanine | Ala | A |
| Cysteine | Cys | C |
| Aspartic Acid | Asp | D |
| Glutamic Acid | Glu | E |
| Phenylalanine | Phe | F |
| Glycine | Gly | G |
| Histidine | His | H |
| Isoleucine | Ile | I |
| Lysine | Lys | K |
| Leucine | Leu | L |
| Methionine | Met | M |
| Asparagine | Asn | N |
| Proline | Pro | P |
| Glutamine | Gln | Q |
| Arginine | Arg | R |
| Serine | Ser | S |
| Threonine | Thr | T |
| Valine | Val | V |
| Tryptophan | Trp | W |
| Tyrosine | Tyr | Y |
| Unknown or other | Xaa | X |

The term “sequence identity” as used herein refers to the amount to amino acids or nucleotides which match exactly between two different sequences. For example, the “percentage of sequence identity” between a first amino acid sequence and a second amino acid sequence may be calculated by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence—compared to the first amino acid sequence—is considered as a difference at a single amino acid residue (i.e. at a single position). Usually, for the purpose of determining the “percentage of sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence. Percent amino acid sequence identity may be determined using the sequence comparison programs known in the art, e.g., NCBI-BLAST.

The term “amino acid difference” as used herein refers to a deletion, insertion or substitution of a single amino acid residue vis-à-vis a reference sequence, and in some embodiments is a substitution.

Such conservative substitutions may be, for example, substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, Ile, Val and Cys; and (e) aromatic residues: Phe, Tyr and Trp.

Exemplary conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

### 5.2 Immunoglobulin Single Variable Domain

The term “immunoglobulin single variable domain” (ISVD), interchangeably used with “single variable domain”, defines immunoglobulin molecules wherein the antigen binding site is present on, and formed by, a single immunoglobulin domain. This sets ISVDs apart from “conventional” immunoglobulins (e.g., monoclonal antibodies) or their fragments (such as Fab, Fab′, F(ab′)2, scFv, di-scFv), wherein two immunoglobulin domains, in particular two variable domains, interact to form an antigen binding site. Typically, in conventional immunoglobulins, a heavy chain variable domain (VH) and a light chain variable domain (VL) interact to form an antigen binding site. In this case, the complementarity determining regions (CDRs) of both VH and VL will contribute to the antigen binding site, i.e., a total of 6 CDRs will be involved in antigen binding site formation.

In view of the above definition, the antigen-binding domain of a conventional 4-chain antibody (such as an IgG, IgM, IgA, IgD or IgE molecule; known in the art) or of a Fab fragment, a F(ab′)2 fragment, an Fv fragment such as a disulfide linked Fv or a scFv fragment, or a diabody (all known in the art) derived from such conventional 4-chain antibody, would normally not be regarded as an ISVD, as, in these cases, binding to the respective epitope of an antigen would normally not occur by one (single) immunoglobulin domain but by a pair of (associating) immunoglobulin domains such as light and heavy chain variable domains, i.e., by a VH-VL pair of immunoglobulin domains, which jointly bind to an epitope of the respective antigen.

In contrast, ISVDs are capable of specifically binding to an epitope of the antigen without pairing with an additional immunoglobulin variable domain. The binding site of an ISVD is formed by a single VH, a single VHH or single VL domain.

As such, the single variable domain may be a light chain variable domain sequence (e.g., a VL-sequence) or a suitable fragment thereof; or a heavy chain variable domain sequence (e.g., a VH sequence or VHH sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit).

An immunoglobulin single variable domain (ISVD) can, for example, be a heavy chain ISVD, such as a VH, VHH, including a camelized VH or humanized VHH. In some embodiments, it is a VHH, including a humanized VHH. In some embodiments it is a VH, including a camelized VH, a human VH, and a camelized human VH. Heavy chain ISVDs can be derived from a conventional four-chain antibody or from a heavy chain antibody.

For example, the ISVD may be a single domain antibody (or an amino acid sequence that is suitable for use as a single domain antibody), a “dAb” or dAb (or an amino acid sequence that is suitable for use as a dAb) or a NANOBODY© ISVD (as defined herein, and including but not limited to a VHH); other single variable domains, or any suitable fragment of any one thereof.

In particular, the ISVD may be a NANOBODY© ISVD (such as a VHH, including a humanized VHH or camelized VH) or a suitable fragment thereof. [Note: NANOBODY© and NANOBODIES© is a registered trademark of Ablynx N.V.]

The term “VHH domain”, also known as VHHs, VHH antibody fragments, and VHH antibodies, have originally been described as the antigen binding immunoglobulin variable domain of “heavy chain antibodies” (i.e., of “antibodies devoid of light chains”; Hamers-Casterman et al. 1993, Nature 363: 446-448). The term “VHH domain” has been chosen in order to distinguish these variable domains from the heavy chain variable domains that are present in conventional 4-chain antibodies (which are referred to herein as “VH domains”) and from the light chain variable domains that are present in conventional 4-chain antibodies (which are referred to herein as “VL domains”). For a further description of VHH's, reference is made to the review article by Muyldermans 2001 (Reviews in Molecular Biotechnology 74: 277-302).

Typically, the generation of immunoglobulins involves the immunization of experimental animals, fusion of immunoglobulin producing cells to create hybridomas and screening for the desired specificities. Alternatively, immunoglobulins can be generated by screening of naïve or synthetic libraries, e.g., by phage display. The generation of immunoglobulin sequences, such as VHHs, has been described extensively in various publications, including WO 94/04678, Hamers-Casterman et al. 1993 (Nature 363: 446-448) and Muyldermans et al. 2001 (Reviews in Molecular Biotechnology 74: 277-302). In these methods, camelids are immunized with the target antigen in order to induce an immune response against said target antigen. The repertoire of VHHs obtained from said immunization is further screened for VHHs that bind the target antigen. In these instances, the generation of antibodies requires purified antigen for immunization and/or screening. Antigens can be purified from natural sources, or by recombinant production approaches. Immunization and/or screening for immunoglobulin sequences can be performed using peptide fragments of such antigens.

Immunoglobulin sequences of different origin, comprising mouse, rat, rabbit, donkey, human and camelid immunoglobulin sequences can be produced, purified and/or isolated. Also, fully human, humanized or chimeric sequences can be produced, purified and/or isolated. For example, camelid immunoglobulin sequences and humanized camelid immunoglobulin sequences, or camelized domain antibodies, e.g., camelized dAb as described by Ward et al. 1989 (Nature 341: 544) (see for example WO 94/04678 and Davies and Riechmann 1994, Febs Lett., 339: 285-290 and 1996, Prot. Eng. 9: 531-537) can be produced, purified and/or isolated. Moreover, the ISVDs are fused to comprise or consist of at least three ISVDs forming a multivalent and/or multispecific construct (for multivalent and multispecific polypeptides containing one or more VHH domains and their preparation, reference is also made to Conrath et al. 2001 (J. Biol. Chem., 276: 7346-7350), as well as to, for example, WO 96/34103 and WO 99/23221. ISVD sequences may further comprise tags or other functional moieties, e.g. toxins, labels, radiochemicals, etc.

The ISVD polypeptide sequence comprised in a polypeptide according to the present technology is not limited as to the origin of the ISVD polypeptide sequence, nor as to the way that the ISVD polypeptide sequence is (or has been) generated or obtained. Thus, the ISVD sequences may be naturally occurring sequences (from any suitable species) or synthetic or semi-synthetic sequences. In a specific but non-limiting aspect, the ISVD sequence is a naturally occurring sequence (from any suitable species) or a synthetic or semi-synthetic sequence, including but not limited to “humanized” immunoglobulin sequences (such as partially or fully humanized mouse or rabbit immunoglobulin sequences, and in particular partially or fully humanized VHH sequences), “camelized” immunoglobulin sequences (and in particular camelized VH sequences), as well as ISVDs that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing.

In one aspect, the ISVD polypeptide sequence may be obtained from mice, rats, rabbits, cats, dogs, goats, sheep, horses, pigs, non-human primates, such as cynomolgus monkeys (also referred to herein as “cyno”), or camelids (such as Llama or Alpaca) or humans.

The term “humanized VHH” comprises an amino acid sequence that corresponds to the amino acid sequence of a naturally occurring VHH domain, but that has been “humanized” by replacing one or more amino acid residues in the amino acid sequence of said naturally occurring VHH sequence (and in particular in the framework sequences) by one or more of the amino acid residues that occur at the corresponding position(s) in a VH domain from a conventional 4-chain antibody from a human being (e.g. indicated above). This can be performed in a manner known per se, which will be clear to the skilled person, for example on the basis of the further description herein and the state of the art (e.g., WO 2008/020079). Again, it should be noted that such humanized VHHs can be obtained in any suitable manner known per se and thus are not strictly limited to polypeptides that have been obtained using a polypeptide that comprises a naturally occurring VHH domain as a starting material.

The term “camelized VH” comprises an amino acid sequence that corresponds to the amino acid sequence of a naturally occurring VH domain, but that has been “camelized” by replacing one or more amino acid residues in the amino acid sequence of a naturally occurring VH domain from a conventional 4-chain antibody by one or more of the amino acid residues that occur at the corresponding position(s) in a VHH domain of a (camelid) heavy chain antibody. This can be performed in a manner known per se, which will be clear to the skilled person, for example based on the further description herein and the state of the art (e.g., Davies and Riechman 1994 and 1996). Such “camelizing” substitutions are inserted at amino acid positions that form and/or are present at the VH-VL interface, and/or at the so-called Camelidae hallmark residues, as defined herein (see for example WO 94/04678 and Davies and Riechmann 1994 and 1996). In some embodiments, the VH sequence that is used as a starting material or starting point for generating or designing the camelized VH is a VH sequence from a mammal, such as the VH sequence of a human being, such as a VH3 sequence. However, it should be noted that such camelized VH can be obtained in any suitable manner known per se and thus are not strictly limited to polypeptides that have been obtained using a polypeptide that comprises a naturally occurring VH domain as a starting material.

The (general) structure of an ISVD comprises four framework regions (“FRs”), which are referred to as “Framework region 1” (“FR1”); as “Framework region 2” (“FR2”); as “Framework region 3” (“FR3”); and as “Framework region 4” (“FR4”); which framework regions are interrupted by three complementary determining regions (“CDRs”), which are referred to as “Complementarity Determining Region 1” (“CDR1”); as “Complementarity Determining Region 2” (“CDR2”); and as “Complementarity Determining Region 3” (“CDR3”). The framework sequences in an ISVD may be any suitable framework sequences, and examples of suitable framework sequences will be clear to the skilled person, for example on the basis of standard handbooks and the further disclosure and literature mentioned herein.

The term “framework sequence” describes (a suitable combination of) immunoglobulin framework sequences or framework sequences that have been derived from immunoglobulin framework sequences (for example, by humanization or camelization). For example, the framework sequences may be framework sequences derived from a light chain variable domain (e.g., a VL-sequence) and/or from a heavy chain variable domain (e.g., a VH-sequence or VHH sequence). In one particular aspect, the framework sequences are either framework sequences that have been derived from a VHH-sequence (in which said framework sequences may optionally have been partially or fully humanized) or are conventional VH sequences that have been camelized (as defined herein).

In particular, an ISVD may be defined as an immunoglobulin sequence with the (general) structure

FR1-CDR1-FR2-CDR2-FR3-CDR3-FR4

wherein “FR1”, “FR2”, FR3”, and “FR4” refer to framework regions 1 to 4; and “CDR1”, “CDR2”, and “CDR3” refer to the complementarity determining regions 1 to 3.

The framework sequences present in the ISVD sequence may contain one or more “hallmark residues”, such that the ISVD sequence is a NANOBODY© ISVD, such as e.g., a VHH, including a humanized VHH, or camelized VH.

The term “hallmark residue” as used herein refers to an amino acid residue that occurs at a defined position in the framework region of a NANOBODY© ISVD.

In particular, an ISVD may be defined as an immunoglobulin sequence with the (general) structure

FR1-CDR1-FR2-CDR2-FR3-CDR3-FR4

wherein “FR1”, “FR2”, FR3”, and “FR4” refer to framework regions 1 to 4; “CDR1”, “CDR2”, and “CDR3” refer to the complementarity determining regions 1 to 3; and wherein the ISVD is further characterized by the presence of one or more “hallmark residues” (see Table 2 below). In some embodiments, the ISVD includes VHH sequences, including (partially) humanized VHH sequences and camelized VH sequences.

For example, an ISVD can be an immunoglobulin sequence with the structure

FR1-CDR1-FR2-CDR2-FR3-CDR3-FR4

wherein “FR1”, “FR2”, FR3”, and “FR4” refer to framework regions 1 to 4; “CDR1”, “CDR2”, and “CDR3” refer to the complementarity determining regions 1 to 3; and wherein the ISVD is further characterized by the presence of one or more of the amino acid residues at positions 11, 37, 44, 45, 47, 83, 84, 103, 104 and 108 according to the Kabat numbering, chosen from the hallmark residues mentioned in Table 2 below.

TABLE 2
Hallmark Residues in an ISVD (see Table A-11 for list of SEQ ID NOs).

| Position | Human VH3 | Hallmark Residues |
|---|---|---|
| 11 | L, V; predominantly L | L, S, V, M, W, F, T, Q, E, A, R, G, K, Y, N, P, I; preferably L |
| 37 | V, I, F; usually V | F(1), Y, V, L, A, H, S, I, W, C, N, G, D, T, P, preferably F⁽¹⁾ or Y |
| 44⁽⁸⁾ | G | E⁽³⁾, Q⁽³⁾, G⁽²⁾, D, A, K, R, L, P, S, V, H, T, N, W, M, I; |
| | | preferably G⁽²⁾, E⁽³⁾ or Q⁽³⁾; most preferably G⁽²⁾ or Q⁽³⁾ |
| 45⁽⁸⁾ | L | L⁽²⁾, R⁽³⁾, P, H, F, G, Q, S, E, T, Y, C, I, D, V; preferably L⁽²⁾ or |
| | | R⁽³⁾ |
| 47⁽⁸⁾ | W, Y | F⁽¹⁾, L⁽¹⁾ or W⁽²⁾ G, I, S, A, V, M, R, Y, E, P, T, C, H, K, Q, N, D; |
| | | preferably W⁽²⁾, L⁽¹⁾ or F⁽¹⁾ |
| 83 | R or K; usually R | R, K⁽⁵⁾, T, E⁽⁵⁾, Q, N, S, I, V, G, M, L, A, D, Y, H; preferably K |
| | | or R; most preferably K |
| 84 | A, T, D; predominantly A | P⁽⁵⁾, S, H, L, A, V, I, T, F, D, R, Y, N, Q, G, E; preferably P |
| 103 | W | W⁽⁴⁾, R⁽⁶⁾, G, S, K, A, M, Y, L, F, T, N, V, Q, P⁽⁶⁾, E, C; preferably |
| | | W |
| 104 | G | G, A, S, T, D, P, N, E, C, L; preferably G |
| 108 | L, M or T; predominantly L | Q, L⁽⁷⁾, R, P, E, K, S, T, M, A, H; preferably Q or L⁽⁷⁾ |

Notes:
⁽¹⁾In particular, but not exclusively, in combination with KERE (SEQ ID NO: 86) or KORE (SEQ ID NO: 87) at positions 43-46.
⁽²⁾Usually as GLEW (SEQ ID NO: 88) at positions 44-47.
⁽³⁾Usually as KERE (SEQ ID NO: 86) or KORE (SEQ ID NO: 87) at positions 43-46, e.g. as KEREL (SEQ ID NO: 89), KEREF (SEQ ID NO: 90), KOREL (SEQ ID NO: 91), KQREF (SEQ ID NO: 92), KEREG (SEQ ID NO: 93), KQREW (SEQ ID NO: 94) or KQREG (SEQ ID NO: 95) at positions 43-47. Alternatively, also sequences such as TERE (SEQ ID NO: 96) (for example TEREL (SEQ ID NO: 97), TORE (SEQ ID NO: 98) (for example TOREL (SEQ ID NO: 99), KECE (SEQ ID NO: 100) (for example KECEL (SEQ ID NO: 101) or KECER (SEQ ID NO: 102), KQCE (SEQ ID NO: 103) (for example KQCEL (SEQ ID NO: 104), RERE (SEQ ID NO: 105) (for example REREG (SEQ ID NO: 106), RQRE (SEQ ID NO: 107) (for example RQREL (SEQ ID NO: 108), RQREF (SEQ ID NO: 109) or RQREW SEQ ID NO: 110), QERE (SEQ ID NO: 111) (for example QEREG (SEQ ID NO: 112), QQRE (SEQ ID NO: 113), (for example QQREW (SEQ ID NO: 114), QQREL (SEQ ID NO: 115) or QQREF (SEQ ID NO: 116), KGRE (SEQ ID NO: 117) (for example KGREG (SEQ ID NO: 118), KDRE (SEQ ID NO: 119) (for example KDREV (SEQ ID NO: 120) are possible. Some other possible, but less preferred sequences, include for example DECKL (SEQ ID NO: 121) and NVCEL (SEQ ID NO: 122).
⁽⁴⁾With both GLEW (SEQ ID NO: 88) at positions 44-47 and KERE (SEQ ID NO: 86) or KORE (SEQ ID NO: 87) at positions 43-46.
⁽⁵⁾Often as KP or EP at positions 83-84 of naturally occurring VHH domains.
⁽⁶⁾In particular, but not exclusively, in combination with GLEW (SEQ ID NO: 88) at positions 44-47.
⁽⁷⁾With the proviso that when positions 44-47 are GLEW (SEQ ID NO: 88), position 108 is always Q in (non-humanized) V_{HH} sequences that also contain a W at 103.
⁽⁸⁾The “GLEW group” (SEQ ID NO: 88) also contains GLEW-like sequences at positions 44-47, such as for example GVEW (SEQ ID NO: 123), EPEW (SEQ ID NO: 124), GLER (SEQ ID NO: 125), DQEW (SEQ ID NO: 126), DLEW (SEQ ID NO: 127), GIEW (SEQ ID NO: 128), ELEW (SEQ ID NO: 129), GPEW (SEQ ID NO: 130), EWLP (SEQ ID NO: 131), GPER (SEQ ID NO: 132), GLER (SEQ ID NO: 133) and ELEW (SEQ ID NO: 134).

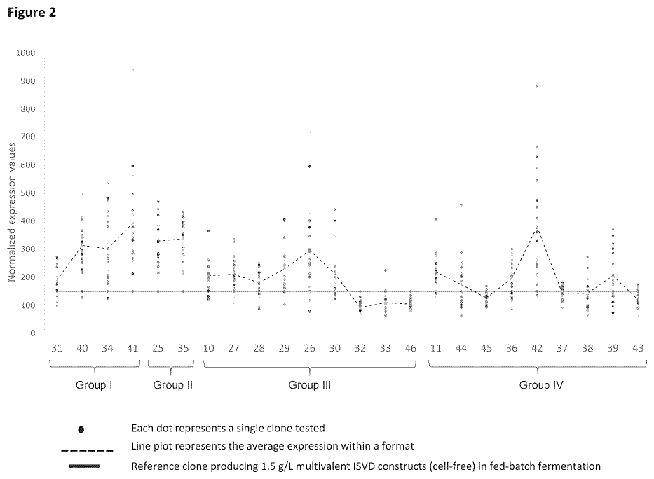

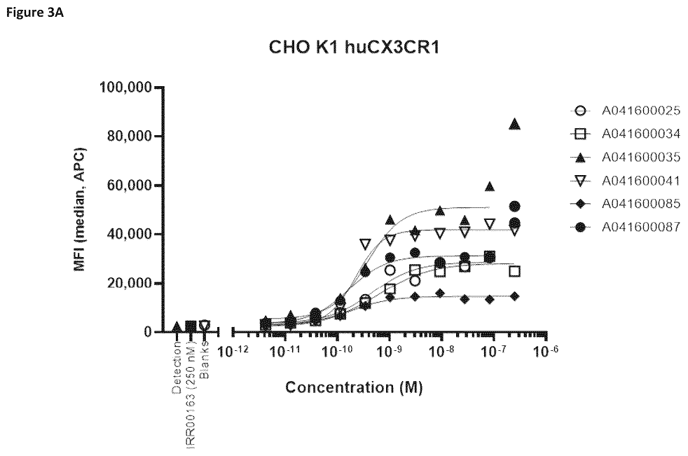

The amino acid residues of an ISVD may be numbered according to the general numbering for VH domains given by Kabat et al. (“Sequence of proteins of immunological interest”, US Public Health Services, NIH Bethesda, MD, Publication No. 91), as applied to VHH domains from Camelids in the article of Riechmann and Muyldermans, 2000 (J. Immunol. Methods 240 (1-2): 185-195; see for example FIG. 2 of this publication) and as further described in paragraph q) on pages 58 and 59 of WO 2008/020079. As is well known in the art for VH domains and for VHH domains, the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. The total number of amino acid residues in a VH domain and a VHH domain will usually be in the range of from 110 to 120, often between 112 and 115. It should however be noted that smaller and longer sequences may also be suitable for the purposes described herein.

In the present application, unless indicated otherwise, FR and CDR sequences of an ISVD (FR1-CDR1-FR2-CDR2-FR3-CDR3-FR4) were determined according to the AbM numbering as described in Kontermann and Dubel (Eds. 2010, Antibody Engineering, vol 2, Springer Verlag Heidelberg Berlin, Martin, Chapter 3, pp. 33-51). According to this method, FR1 comprises the amino acid residues at positions 1-25, CDR1 comprises the amino acid residues at positions 26-35, FR2 comprises the amino acids at positions 36-49, CDR2 comprises the amino acid residues at positions 50-58, FR3 comprises the amino acid residues at positions 59-94, CDR3 comprises the amino acid residues at positions 95-102, and FR4 comprises the amino acid residues at positions 103-113.

Determination of CDR regions may also be done according to different methods (e.g., Kabat, Chothia or IMGT). For example, in the CDR determination according to Kabat, FR1 of an ISVD comprises the amino acid residues at positions 1-30, CDR1 of an ISVD comprises the amino acid residues at positions 31-35, FR2 of an ISVD comprises the amino acids at positions 36-49, CDR2 of an ISVD comprises the amino acid residues at positions 50-65, FR3 of an ISVD comprises the amino acid residues at positions 66-94, CDR3 of an ISVD comprises the amino acid residues at positions 95-102, and FR4 of an ISVD comprises the amino acid residues at positions 103-113.

The ISVD has a “three-dimensional structure”. The term “three-dimensional structure” in the context of an ISVD for example, refers to a complex structure comprising α-helices and β-sheets folded into a compact structure that is stabilized by both polar and nonpolar interactions. The three-dimensional structure forms spontaneously and is maintained as a result of interactions among the side chains of the amino acids.

The crystallographic structure of different VHHs has been reported (Desmyter et al. 1996, Nat. Struct. Biol. 3: 803-811; Spinelli et al. 1996, Nature Struct Biol 3: 752; Decanniere et al. 1999, Structure 7: 361-370). VHHs adopt the standard fold of an immunoglobulin variable domain. The immunoglobulin variable domain folds into a native conformation, also viewed as a beta-barrel. The FR regions of the immunoglobulin variable domain form nine parallel beta-strands folded in two sheets that pack against each other and are stabilised by a conserved disulfide bond making up a beta-sheet scaffold. The antigen binding site formed by the CDR loops (hypervariable regions) indeed top on this core scaffold structure, clustering at one end of the domain.

The present technology provides improved ISVDs that bind CX3CR1. The ISVDs of the present technology are based on ISVD 66B02 (SEQ ID NO: 4) and 54A12 (SEQ ID NO: 1) described in WO 2013/130381 (SEQ ID NOs: 1 and 2 of WO 2013/130381).

In one aspect, the sequence optimised ISVD has the general structure FR1-CDR1-FR2-CDR2-FR3-CDR3-FR4; and wherein the ISVD specifically binds to fractalkine receptor CX3CR1 and/or a single nucleotide polymorphism (SNP) variant of CX3CR1 and comprises (according to AbM numbering)
a CDR1 comprising or consisting of the amino acid sequence GSIFSSTAMA (SEQ ID NO: 10);
a CDR2 comprising the amino acid sequence X₁IX₃TVGX₇TK (SEQ ID NO: 202), wherein the ambiguity residues X may be X₁=A or V, X₃═N or S, X₇═I or V; and
a CDR3 comprising the amino acid sequence DX₁RRGWDTRY (SEQ ID NO: 138), wherein the ambiguity residue X may be X₁═P or A; and

In some embodiments, CDR2 comprises the amino acid sequence X₁IX₃TVGX₇TK (SEQ ID NO: 202), wherein X₁=A, X₃═N or S, X₇═I or V; X₁=A or V, X₃═N, X₇═I or V; X₁=A or V; X₃═N or S; X₇═I; X₁=A, X₃═N, X₇═I or V; X₁=A, X₃═N or S, X₇═I or V; or X₁=A or V, X₃═N, X₇═I.

In some embodiments, CDR2 comprises the amino acid sequence VISTVGITK (SEQ ID NO: 12).

In some embodiments, CDR3 comprises the amino acid sequence DARRGWDTRY (SEQ ID NO: 14).

In some embodiments, the ISVDs were sequence optimized resulting in 66B02_SO (SEQ ID NO: 5) and 54A12_SO (SEQ ID NO: 2).

The sequence optimized ISVDs, such as 66B02_SO (SEQ ID NO: 5) and 54A12_SO (SEQ ID NO: 2), are improved compared to ISVD 66B02 (SEQ ID NO: 4) and 54A12 (SEQ ID NO: 1) in at least one of the follow characteristics:
more human-like (humanization);
reduced binding by pre-existing antibodies;
reduced antigenicity (less T-cell epitopes);
improved chemical stability

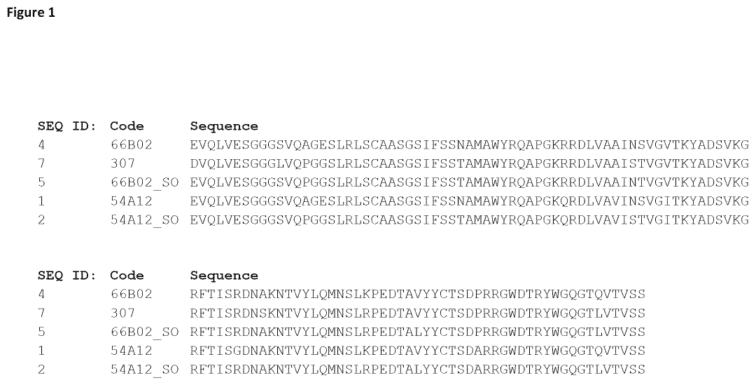

The sequences of the CDR regions and the FR regions of exemplary sequence optimized ISVDs are depicted in Table A-2 (CDR determination based on AbM) and Table A-3 (CDR determination based on Kabat). Alignment of the ISVD sequences described in WO 2013/130381 and exemplary ISVDs that were sequence optimized in the present technology is given in FIG. 1.

### 5.3 Polypeptide

A polypeptide according to the present technology may comprise or consist of various ISVDs, also referred to herein as ISVD building blocks, such as a “first ISVD”, “second ISVD” and a “third ISVD”. In some cases, the terms “first ISVD”, “second ISVD” and “third ISVD” may indicate the relative position of the specifically recited ISVDs to each other, wherein the numbering is started from the N-terminus of the polypeptide. The “first ISVD” is thus closer to the N-terminus than the “second ISVD” and “third ISVD”. Accordingly, the “third ISVD” is thus closer to the C-terminus than the “first ISVD” and “second ISVD”. Since the numbering is thus not absolute and only indicates the relative position of the two ISVDs, it does not exclude the possibility that additional binding units/building blocks can be present in the polypeptide. Moreover, it does not exclude the possibility that other binding units/building blocks such as ISVDs can be placed in between.

ISVD building blocks in a polypeptide according to the present technology may be linked directly or via a peptidic linker. The term “directly” in this context refers to two ISVDs comprising an immunoglobulin sequence with the structure

[FR1-CDR1-FR2-CDR2-FR3-CDR3-FR4]_{ISVD1}-L-[FR1-CDR1-FR2-CDR2-FR3-CDR3-FR4]_{ISVD2}

wherein “FR1”, “FR2”, FR3”, and “FR4” refer to framework regions 1 to 4; “CDR1”, “CDR2”, and “CDR3” refer to the complementarity determining regions 1 to 3. and “L” between ISVD1 and ISVD2 refers to a single peptidic bond to connect the ISVDs.

Linkage of the two ISVDs via a “peptidic linker” refers to two ISVDs comprising an immunoglobulin sequence with the structure

[FR1-CDR1-FR2-CDR2-FR3-CDR3-FR4]_{ISVD1}-L-[FR1-CDR1-FR2-CDR2-FR3-CDR3-FR4]_{ISVD2}

wherein “FR1”, “FR2”, FR3”, and “FR4” refer to framework regions 1 to 4; “CDR1”, “CDR2”, and “CDR3” refer to the complementarity determining regions 1 to 3, and “L” between ISVD1 and ISVD2 refers to a linker to connect the ISVDs (e.g., as described in Table A-7).

Peptidic linkers may be synthetic amino acid sequences that consist of a linear chain of amino acids and can have a length of, for example, 1 to 62 amino acid residues, or 1 to 50 amino acid residues, preferably with a length of 9 to 40 amino acid residues. The linker may ensure that the ISVDs connected by the linker can perform their biological activity. In some cases, the linker comprises alanine, glycine, and/or serine residues, e.g., arranged in repetitive units. In the present technology, ISVDs may be linked via a peptidic linker, but may also be directly linked without a peptidic linker.

Some preferred examples of such amino acid linker sequences include Gly-Ser linkers, for example of the type (GlyₓSer_{y})_{z}, such as (for example (Gly₄Ser)₃ (SEQ ID NO: 71) or (Gly₃Ser₂)₃ (SEQ ID NO: 136), as described in WO 99/42077, hinge-like regions such as the hinge regions of naturally occurring heavy chain antibodies or similar sequences (such as described in WO 94/04678). Gly-Ser linkers comprise one or more repeats of a peptide motif such as the GGGGS (SEQ ID NO: 66) motif (for example, have the formula (Gly-Gly-Gly-Gly-Ser)n (SEQ ID NO: 66) in which n may be 1, 2, 3, 4, 5, 6, 7 or more). Some often-used examples of such GS linkers are 9GS linkers (GGGGSGGGS, SEQ ID NO: 69), 15GS linkers (n=3; SEQ ID NO: 71) and 35GS linkers (n=7; SEQ ID NO: 76). Reference is for example made to Chen et al. 2013 (Adv. Drug Deliv. Rev. 65: 1357-1369) and Klein et al. 2014 (Protein Eng. Des. Sel. 27: 325-330). In some embodiments, 9GS linkers (SEQ ID NO: 69) are used to link the ISVDs in the polypeptide to each other.

For example, a polypeptide according to the present technology may comprise two peptidic linkers located between two ISVDs within the polypeptide. For example, a polypeptide may comprise an immunoglobulin sequence with the structure

[FR1-CDR1-FR2-CDR2-FR3-CDR3-FR4]_{ISVD1}-L1-[FR1-CDR1-FR2-CDR2-FR3-CDR3-FR4]_{ISVD2}L2-[FR1-CDR1-FR2-CDR2-FR3-CDR3-FR4]_{ISVD3}

wherein “FR1”, “FR2”, FR3”, and “FR4” refer to framework regions 1 to 4; “CDR1”, “CDR2”, and “CDR3” refer to the complementarity determining regions 1 to 3, and “L1” and “L2” between two ISVDs within the polypeptide refer to a linker to connect the ISVDs (e.g., as described in Table A-7). In some embodiments, L1 and L2 are SEQ ID NO: 69.

The term “monovalent” indicates the presence of only one binding units/building block such as one ISVD, in the polypeptide. The term “monospecific” refers to the binding to one (specific) type of target molecule.

The terms “bivalent”, “trivalent”, “tetravalent” or “pentavalent” all fall under the term “multivalent” and indicate the presence of two, three, four or five binding units (such as ISVDs). The terms “bispecific”, “trispecific”, “tetraspecific” or “pentaspecific” all fall under the term “multispecific” and refer to binding to two, three, four or five different target molecules, respectively.

In one aspect, a polypeptide comprises at least three ISVDs wherein each ISVD has the general structure FR1-CDR1-FR2-CDR2-FR3-CDR3-FR4; and wherein the first ISVD and the second ISVD specifically bind to fractalkine receptor CX3CR1 and/or a single nucleotide polymorphism (SNP) variant of CX3CR1 and comprise (according to AbM numbering)
a CDR1 comprising or consisting of the amino acid sequence GSIFSSTAMA (SEQ ID NO: 10);
a CDR2 comprising the amino acid sequence X₁IX₃TVGX₇TK (SEQ ID NO: 202), wherein the ambiguity residues X may be X₁=A, V; X₃═N, S; X₇=1, V; and
a CDR3 comprising the amino acid sequence DX₁RRGWDTRY (SEQ ID NO: 138), wherein the ambiguity residue X may be X₁═P, A; and
   wherein the third ISVD specifically binds to albumin and comprises (according to AbM numbering)
a CDR1 as shown in GFTFRSFGMS (SEQ ID NO: 53) or having two or one amino acid difference(s) with SEQ ID NO: 53;
a CDR2 as shown in SISGSGSDTL (SEQ ID NO: 55) or having two or one amino acid difference(s) with SEQ ID NO: 55; and
a CDR3 as shown in GGSLSR (SEQ ID NO: 57) or having two or one amino acid difference(s) with SEQ ID NO: 57; and
   wherein the at least three ISVDs are linked directly or via a peptidic linker.

In some embodiments, the first ISVD and the second ISVD comprise a CDR2 comprising the amino acid sequence X₁IX₃TVGX₇TK (SEQ ID NO: 202), wherein X₁=A, X₃═N or S, X₇═I or V; X₁=A or V, X₃═N, X₇═I or V; X₁=A or V; X₃═N or S; X₇═I; X₁=A, X₃═N, X₇═I or V; X₁=A, X₃═N or S, X₇═I or V; or X₁=A or V, X₃═N, X₇═I.

In some embodiments, CDR2 comprises the amino acid sequence VISTVGITK (SEQ ID NO: 12).

In some embodiments, CDR3 comprises the amino acid sequence DARRGWDTRY (SEQ ID NO: 14).

The combination of CDR1, CDR2 and CDR3 of the first ISVD and/or the second ISVD may be selected from the following embodiments, wherein exemplary combinations of CDR1, CDR2 and CDR3 are represented by one row of Table 3.

TABLE 3
CDRs 1 to 3 of the ISVD specifically binding to
fractalkine receptor CX3CR1 and/or a single nucleotide
polymorphism (SNP) variant of CX3CR1.

| | CDR1 | CDR2 | CDR3 |
|---|---|---|---|
| | (SEQ ID NO) | (SEQ ID NO) | (SEQ ID NO) |
| 54A12_SO (AbM) | 10 | 12 | 14 |
| 66B02_SO (AbM) | 10 | 19 | 21 |
| 54A12_SO (Kabat) | 24 | 26 | 14 |
| 66B02_SO (Kabat) | 24 | 33 | 21 |

In one aspect, the amino acid sequence of the ISVD binding to fractalkine receptor CX3CR1 and/or a single nucleotide polymorphism (SNP) variant of CX3CR1 may have a sequence identity of 90% or more (such as 91%, 92%, 93%, 94% or more), 95% or more (such as 96%, 97%, 98% or more) or even 99% or more with any of the amino acid sequences of SEQ ID NOs 1 to 8. While the CDRs of the ISVD may have three, two or one amino acid difference(s) as compared to the specific CDRs shown herein, they retain the biological activity in terms of efficacy and potency. For example, ISVDs having variability in the CDRs of 3, 2 or 1 amino acid as compared to the specific CDRs shown herein retain their binding specificity to fractalkine receptor CX3CR1 and/or a single nucleotide polymorphism (SNP) variant of CX3CR1.

In some embodiments, the ISVDs specifically binding to CX3CR1 are positioned at the N-terminus of the polypeptide. Accordingly, in these embodiments, the polypeptide comprises or consists of the following, in order starting from the N-terminus of the polypeptide: an ISVD specifically binding to CX3CR1, an ISVD specifically binding to CX3CR1, an ISVD providing the polypeptide with increased half-life. The inventors surprisingly found that such a configuration increased the expression yield of the polypeptide. Moreover, such configuration of the polypeptide provided good chemistry, manufacturing, and controls (CMC) characteristics, including upstream and downstream manufacturability. CMC activities are important activities when developing new pharmaceutical products. It involves defining manufacturing practices and product specifications that must be followed and met in order to ensure product safety and consistency between batches.

In one aspect, a polypeptide comprises at least three ISVDs wherein each ISVD has the general structure FR1-CDR1-FR2-CDR2-FR3-CDR3-FR4; wherein the first ISVD and the second ISVD specifically bind to fractalkine receptor CX3CR1 and/or a single nucleotide polymorphism (SNP) variant of CX3CR1 (such as human fractalkine receptor CX3CR1) and the third ISVD specifically binds to albumin (such as human serum albumin); wherein the at least three ISVDs are linked directly or via a peptidic linker (L1, L2); wherein the C-terminal ISVD of the polypeptide carries a C-terminal extension of FR4 comprising 1, 2, 3, 4, 5, 6, 7, 8, 9 or 10 amino acids in length; and wherein the order of ISVDs and peptidic linkers as recited in Table 4 below indicates their relative position to each other within the polypeptide, considered from the N-terminus (first column) to the C-terminus (last column) of said polypeptide.

TABLE 4
Exemplary polypeptides of the present technology.

| Polypeptide | ISVD 1 | L1 | ISVD 2 | L2 | ISVD 3 |
|---|---|---|---|---|---|
| (SEQ ID | (SEQ ID | (SEQ ID | (SEQ ID | (SEQ ID | (SEQ ID |
| NO) | NO) | NO) | NO) | NO) | NO) |
| 176 | 5 | 69 | 37 | 69 | 5 |
| 177 | 5 | 69 | 5 | 69 | 37 |
| 178 | 2 | 69 | 37 | 69 | 2 |
| 179 | 5 | 73 | 37 | 73 | 5 |
| 180 | 5 | 69 | 37 | 73 | 5 |
| 181 | 5 | 69 | 37 | 76 | 5 |
| 182 | 5 | 73 | 37 | 69 | 5 |
| 183 | 5 | 73 | 37 | 76 | 5 |
| 184 | 5 | 69 | 37 | 69 | 2 |
| 185 | 5 | 76 | 37 | 69 | 5 |
| 186 | 5 | 76 | 37 | 73 | 5 |
| 187 | 2 | 69 | 37 | 69 | 5 |
| 188 | 2 | 69 | 2 | 69 | 37 |
| 189 | 5 | 73 | 5 | 69 | 37 |
| 190 | 5 | 73 | 5 | 76 | 37 |
| 191 | 5 | 76 | 5 | 69 | 37 |
| 192 | 5 | 76 | 5 | 73 | 37 |
| 193 | 5 | 69 | 2 | 69 | 37 |
| 194 | 2 | 69 | 5 | 69 | 37 |
| 195 | 5 | 73 | 5 | 73 | 37 |
| 196 | 5 | 76 | 5 | 76 | 37 |
| 197 | 5 | 69 | 5 | 73 | 37 |
| 198 | 5 | 69 | 5 | 76 | 37 |
| 199 | 5 | 76 | 37 | 76 | 5 |
| 153 | 6 | 69 | 37 | 69 | 5 |
| 154 | 6 | 69 | 5 | 69 | 37 |
| 155 | 3 | 69 | 37 | 69 | 2 |
| 156 | 6 | 73 | 37 | 73 | 5 |
| 157 | 6 | 69 | 37 | 73 | 5 |
| 158 | 6 | 69 | 37 | 76 | 5 |
| 159 | 6 | 73 | 37 | 69 | 5 |
| 160 | 6 | 73 | 37 | 76 | 5 |
| 161 | 6 | 69 | 37 | 69 | 2 |
| 162 | 6 | 76 | 37 | 69 | 5 |
| 163 | 6 | 76 | 37 | 73 | 5 |
| 164 | 3 | 69 | 37 | 69 | 5 |
| 82 | 3 | 69 | 2 | 69 | 37 |
| 165 | 6 | 73 | 5 | 69 | 37 |
| 166 | 6 | 73 | 5 | 76 | 37 |
| 167 | 6 | 76 | 5 | 69 | 37 |
| 168 | 6 | 76 | 5 | 73 | 37 |
| 169 | 6 | 69 | 2 | 69 | 37 |
| 170 | 3 | 69 | 5 | 69 | 37 |
| 171 | 6 | 73 | 5 | 73 | 37 |
| 172 | 6 | 76 | 5 | 76 | 37 |
| 173 | 6 | 69 | 5 | 73 | 37 |
| 174 | 6 | 69 | 5 | 76 | 37 |
| 175 | 6 | 76 | 37 | 76 | 5 |

In one aspect, the amino acid sequence of a polypeptide comprises ISVD1 directly followed by L1 directly followed by ISVD2 directly followed by L2 directly followed by ISVD3 (as described in one row of Table 4) and, optionally directly followed by a C-terminal extension of FR4 of ISVD3 (e.g., one, two or three alanine (Ala) residue(s)); and the polypeptide may have a sequence identity of 90% or more (such as 91%, 92%, 93%, 94% or more), 95% or more (such as 96%, 97%, 98% or more) or even 99% or more with with any of the amino acid sequences of the polypeptides represented by one row of Table 4. For example, a polypeptide having 10, 9, 8, 7, 6, 5, 4, 3, 2 or 1 amino acid(s) variability in the amino acid sequence as compared to the specific amino acid sequence (e.g., SEQ ID NO: 82) shown herein retain their good CMC characteristics, including upstream and downstream manufacturability and/or their biological activity in terms of efficacy and potency.

In one aspect, a polypeptide comprises three ISVDs (1) to (3) and two peptidic linkers, wherein each peptidic linker is located between two ISVDs within the polypeptide; wherein the order of (1), (2), and (3) indicates their relative position to each other within the polypeptide, considered from the N-terminus to the C-terminus of said polypeptide; and
wherein ISVDs (1) and (2) specifically bind to fractalkine receptor CX3CR1 and/or a single nucleotide polymorphism (SNP) variant of CX3CR1 and ISVDs (1) and (2) comprise (according to AbM numbering) a CDR1 as shown in GSIFSSTAMA (SEQ ID NO: 10); a CDR2 as shown in VISTVGITK (SEQ ID NO: 12); and a CDR3 as shown in DARRGWDTRY (SEQ ID NO: 14);
wherein the third ISVD specifically binds to albumin and comprises (according to AbM numbering) a CDR1 as shown in GFTFRSFGMS (SEQ ID NO: 53); a CDR2 as shown in SISGSGSDTL (SEQ ID NO: 55); and a CDR3 as shown in GGSLSR (SEQ ID NO: 57); and wherein the third ISVD has a C-terminal extension of 1 to 3 alanine or 1 to 3 glycine residues.

Determination of CDR regions may also be done according to Kabat. Accordingly, the present technology also provides a polypeptide comprising or consisting of at least three immunoglobulin single variable domains (ISVDs), wherein each of the at least three ISVDs has the structure FR1-CDR1-FR2-CDR2-FR3-CDR3-FR4 in which FR1 to FR4 refer to framework regions and CDR1 to CDR3 refer to complementarity determining regions; and wherein the polypeptide comprises
(a) a first ISVD specifically binding to fractalkine receptor CX3CR1 and/or a single nucleotide polymorphism (SNP) variant of CX3CR1 and comprising (according to Kabat numbering)
a CDR1 as shown in SEQ ID NO: 24;
a CDR2 as shown in SEQ ID NO: 26; and
a CDR3 as shown in SEQ ID NO: 14; and
(b) a second ISVD specifically binding to fractalkine receptor CX3CR1 and/or a single nucleotide polymorphism (SNP) variant of CX3CR1 and comprising (according to Kabat numbering)
a CDR1 as shown in SEQ ID NO: 24;
a CDR2 as shown in SEQ ID NO: 26; and
a CDR3 as shown in SEQ ID NO: 14; and
(c) a third ISVD specifically binding to albumin and comprising (according to Kabat numbering)
a CDR1 as shown in SEQ ID NO: 60 or having two or one amino acid difference(s) with SEQ ID NO: 53;
a CDR2 as shown in SEQ ID NO: 62 or having two or one amino acid difference(s) with SEQ ID NO: 55; and
a CDR3 as shown in SEQ ID NO: 57 or having two or one amino acid difference(s) with SEQ ID NO: 57; and
wherein (a), (b), and (c) are linked directly or via a peptidic linker.

In some embodiments, the C-terminal ISVD of the polypeptide as described herein carries a C-terminal extension of FR4 comprising 1, 2, 3, 4, 5, 6, 7, 8, 9 or 10 amino acids in length.

In some embodiments, the order of (a), (b), and (c) indicates the relative position of the ISVDs to each other within the polypeptide, considered from the N-terminus to the C-terminus of said polypeptide.

In some embodiments, the peptidic linker comprises 3 to 62 amino acids. The amino acids in the peptide linker may be selected from alanine (A), glycine (G) and serine (S). In a specific aspect, the peptidic linker is selected from a 9 GS linker, a 20 GS linker and a 35 GS linker, such as e.g., SEQ ID NO: 69.

The term “C-terminal extension” refers to a C-terminal extension (tail) relative to the ISVD sequence. In particular, the C-terminal ISVD of the polypeptide may carry a C-terminal extension of FR4 comprising 1, 2, 3, 4, 5, 6, 7, 8, 9 or 10 amino acids in length. The C-terminal extension comprises an amino acid sequence not naturally associated with an ISVD sequence (i.e. FR1-CDR1-FR2-CDR2-FR3-CDR3-FR4).

In some embodiments the ISVD at the C-terminal end of the polypeptide comprises a C-terminal extension (X)ₙ, in which n is 1 to 10, or 1 to 5, such as 1, 2, 3, 4 or 5 (and including 1 or 2, such as 1); and each X is an (in some embodiments, naturally occurring) amino acid residue that is independently chosen, and may independently be chosen from the group consisting of alanine (A), glycine (G), valine (V), leucine (L) and isoleucine (I). In an embodiment, the n is 1 and X is alanine (A).

In some embodiments, the polypeptide comprises an amino acid sequence having a sequence identity of 90% or more (such as 91%, 92%, 93%, 94% or more), 95% or more (such as 96%, 97%, 98% or more) or even 99% or more with SEQ ID NO: 82. In some embodiments the polypeptide comprises SEQ ID NO: 82. In some embodiments the polypeptide consists of SEQ ID NO: 82.

The term “affinity”, represented by the equilibrium constant for the dissociation of an antigen with an antigen-binding protein (K_{D}), as used herein, refers to the binding strength between an antigenic determinant and an antigen-binding site on the antigen-binding protein: the lesser the value of the K_{D}, the stronger the binding strength between an antigenic determinant and the antigen-binding molecule (alternatively, the affinity can also be expressed as the affinity constant (K_{A}), which is 1/K_{D}). Affinity can be determined in a manner known per se, depending on the specific antigen of interest. The affinity of a molecular interaction between two molecules can be measured via different techniques known per se, such as the well-known surface plasmon resonance (SPR) biosensor technique (see for example Ober et al. 2001, Intern. Immunology 13: 1551-1559). The term “surface plasmon resonance”, as used herein, refers to an optical phenomenon that allows for the analysis of real-time biospecific interactions by detection of alterations in protein concentrations within a biosensor matrix, where one molecule is immobilized on the biosensor chip and the other molecule is passed over the immobilized molecule under flow conditions yielding kₒₙ, k_{off} measurements and hence K_{D} (or K_{A}) values. This can for example be performed using the well-known BIAcore® system (BIAcore International AB, a GE Healthcare company, Uppsala, Sweden and Piscataway, NJ). For further descriptions, see Jonsson et al. (1993, Ann. Biol. Clin. 51: 19-26), Jonsson et al. (1991 Biotechniques 11: 620-627), Johnsson et al. (1995, J. Mol. Recognit. 8: 125-131), and Johnnson et al. (1991, Anal. Biochem. 198: 268-277).

Another well-known biosensor technique to determine affinities of biomolecular interactions is bio-layer interferometry (BLI) (see for example Abdiche et al. 2008, Anal. Biochem. 377: 209-217). The term “bio-layer Interferometry” or “BLI”, as used herein, refers to a label-free optical technique that analyzes the interference pattern of light reflected from two surfaces: an internal reference layer (reference beam) and a layer of immobilized protein on the biosensor tip (signal beam). A change in the number of molecules bound to the tip of the biosensor causes a shift in the interference pattern, reported as a wavelength shift (nm), the magnitude of which is a direct measure of the number of molecules bound to the biosensor tip surface. Since the interactions can be measured in real-time, association and dissociation rates and affinities can be determined. BLI can for example be performed using the well-known Octet® Systems (ForteBio, a division of Pall Life Sciences, Menlo Park, USA).

Alternatively, affinities can be measured in Kinetic Exclusion Assay (KinExA) (see for example Drake et al. 2004, Anal. Biochem. 328: 35-43), using the KinExA© platform (Sapidyne Instruments Inc, Boise, USA). The term “KinExA”, as used herein, refers to a solution-based method to measure true equilibrium binding affinity and kinetics of unmodified molecules. Equilibrated solutions of an antibody/antigen complex are passed over a column with beads precoated with antigen (or antibody), allowing the free antibody (or antigen) to bind to the coated molecule. Detection of the antibody (or antigen) thus captured is accomplished with a fluorescently labeled protein binding the antibody (or antigen).

The GYROLAB® immunoassay system provides a platform for automated bioanalysis and rapid sample turnaround (Fraley et al. 2013, Bioanalysis 5: 1765-74).

Meso Scale Discovery-Solution Equilibrium Titration (MSD-SET) is a high-throughput procedure that allows reliable affinity screening of unpurified antibody fragments (Estep P. et al. 2013 MAbs. 5(2): 270-8).

The terms “specificity”, “binding specifically” or “specific binding” refer to the number of different target molecules, such as antigens, from the same organism to which a particular binding unit, such as an ISVD, can bind with sufficiently high affinity (see below). In some instances, also “selectivity”, “binding selectively” or “selective binding” are used in this context. Binding units, such as ISVDs, specifically bind to their designated targets. The specificity/selectivity of a binding unit can be determined based on affinity. The affinity denotes the strength or stability of a molecular interaction. The affinity is commonly given as by the KD, or dissociation constant, which has units of mol/liter (or M). The affinity can also be expressed as an association constant, KA, which equals 1/KD and has units of (mol/liter)⁻¹ (or M⁻¹).

The term “fractalkine receptor CX3CR1” as used herein refers to C-X3-C motif chemokine receptor 1 (CX3CR1), also known as the fractalkine receptor or G-protein coupled receptor 13 (GPR13), which is a transmembrane protein and a receptor for the chemokine fractalkine (also known as CX3CL1).

In some embodiments, the human CX3CR1 and cynomolgus CX3CR1 comprises an amino acid sequence having a sequence identity of more than 90% or a sequence identity of more than 95% with SEQ ID Nos: 84 and 85 (Table A-9).

In some embodiments, the human CX3CR1 and cynomolgus CX3CR1 are as depicted in SEQ ID NOs: 84 and 85, respectively (Table A-9).

The term “single nucleotide polymorphism (SNP) variant” as used herein refers to an allelic variant of a gene (e.g., CX3CR1) containing at least one single nucleotide polymorphism (SNP). “SNP” means a single nucleotide variation between the genomes of individuals of the same species. In some cases, a SNP may be a single nucleotide deletion or insertion. In general, SNPs occur relatively frequently in genomes and thus contribute to genetic diversity. SNPs are thought to be mutationally more stable than other polymorphisms, lending their use in association studies in which linkage disequilibrium between markers and an unknown variant is used to map disease-causing mutations. SNPs may have two, three or four alleles, or (although it may be possible to have three or four different forms of an SNP, corresponding to the different nucleotides), thus facilitating genotyping (by a simple plus/minus assay rather than a length measurement) and automation. The location of a SNP is generally flanked by highly conserved sequences. An individual may be homozygous or heterozygous for an allele at each SNP site. A heterozygous SNP allele can be a differentiating polymorphism. SNPs may occur in protein-coding nucleic acid sequences (a “cSNP”). Such a SNP may result in an amino acid change in the encoded protein which may have functional consequences i.e., result in a “variant” protein or polypeptide. Alternatively, such a SNP may be “silent” in that it does not result in an amino acid change.

SNPs may also occur in introns and in intergenic regions but may result in a phenotypic change. For example, a SNP resulting in aberrant splicing may result in a non-functional protein. Alternatively, a SNP may have no phenotypic effect. A variant protein or polypeptide contains at least one amino acid residue that differs from the corresponding amino acid sequence of the polypeptide that is referred to as “wild-type” or “normal” in the art. Such variant polypeptides can result from a codon change or from a nonsense mutation, or from any SNP that results in altered structure, function, activity, regulation, or expression of a protein.

In order to identify potential SNPs, a subject's genome (e.g., of a patient suffering from a CX3CR1-related disease or condition) may be sequenced and analysed (e.g., by SNP genotyping methods). In addition, SNPs can be derived from dbSNP database (www.ncbi.nlm.nih.gov/snp/) wherein they get a unique dbSNP Reference SNP (rs or RefSNP) number assigned for locus accession for a variant type.

For example, a single nucleotide polymorphism (SNP) variant of CX3CR1 may comprise at least one SNP selected from the group comprising or consisting of SNPs rs3732378 (also known as rs60081475, rs52789411, rs17792900), and rs3732379 (also known as rs59717546, rs52808794, rs17792918) (sbSNP database, https://www.ncbi.nlm.nih.gov/snp, “CX3CR1” and clinical significance=“pathogenic”), or selected from the group comprising or consisting of rs17038679, rs41535248, rs55975803, rs56181422, rs139019894, rs199811198, rs201442030, rs202143296, rs376411124, rs1575205636, (sbSNP database, https://www.ncbi.nlm.nih.gov/snp, “CX3CR1” and clinical significance=“benign” or “likely benign”), rs938203, rs2669849 and rs1050592 (Tremblay et al. 2006, Genes Immun. 7: 632-9).

The term “albumin”, or “serum albumin” refers to a family of globular proteins which are commonly found in blood plasma of animals and humans. Serum albumin is encoded by the albumin gene. Preferably, albumin is human serum albumin. For example, human serum albumin as described in NCBI Reference Sequence: NP_000468.1.

In some embodiments, the polypeptide as described herein has a lower affinity and/or does not bind cell surface glycoprotein MUC18 (alternative name: Melanoma Cell Adhesion Molecule (MCAM); https://www.uniprot.org/uniprot/P43121). In some embodiments, the polypeptide as described herein binds Melanoma Cell Adhesion Molecule (MCAM) with a low affinity (e.g., a K_{D} of 10⁻⁴ M or lower, such as 10⁻³ M, 10⁻² or even lower). In some embodiments, the polypeptide as described herein binds Melanoma Cell Adhesion Molecule (MCAM) with a lower affinity compared to the binding to MCAM by A041600087 (SEQ ID NO: 83), e.g., as measured by antibody specificity profiling (e.g. by a protein microarray).

The term “antigenicity” as used herein in the context of a polypeptide, refers to the presence of B cell or T cell epitopes on the polypeptide. “Antigenicity” describes the ability of the polypeptide (antigen) to bind to, or interact with, the products of the final cell-mediated response such as B-cell or T-cell receptors. Antigenic determinants, or epitopes, are structural features on the polypeptide (antigen) that interact with B-cell receptors, also known as antibodies or immunoglobulins. T-cell receptors recognize linear amino acid sequences within a polypeptide antigen, also referred to as epitopes, when they combine with a major histocompatibility complex (MHC) molecule. For example, antigenicity may be assessed by the binding on Human Leukocyte Antigen DR isotype (HLA-DR) alleles.

In some embodiments, the polypeptide as described herein has a lower antigenicity compared to A041600087 (SEQ ID NO: 83), e.g. as measured by the binding on Human Leukocyte Antigen DR isotype (HLA-DR) alleles (e.g. with NetMHCllpan-v4.0 software tool (http://www.cbs.dtu.dk/services/NetMHCllpan/)).

The term “pre-existing antibodies” refers to antibodies that are not “provoked” or “induced” by administration of a drug, such as by an ISVD containing polypeptide (as is the case with anti-drug antibodies (ADAs)). “Pre-existing antibodies” are already present in blood or serum of subjects that have never received any drug, such as an ISVD containing polypeptide.

The polypeptide of the present technology may exhibit limited reactivity to pre-existing antibodies (i.e. antibodies present in the subject before the first treatment with the antibody construct) in the subject to be treated.

### 5.4 A Polynucleotide, a Vector and a Host

The term “polynucleotide” or “nucleic acid molecule” as used herein refers to a linear polymer composed of covalently linked natural or non-natural nucleotide sequence comprising deoxyribonucleic acid (DNA), ribonucleic acid (RNA) of a combined DNA-RNA. In particular, nucleotide sequences may be a naturally occurring nucleotide sequence, a (chemically) modified nucleotide sequence, or a synthetic or semi-synthetic nucleotide sequence, and may for example be sequences that are isolated by PCR from a suitable naturally occurring template (e.g., DNA or RNA isolated from a cell), nucleotide sequences that have been isolated from a library (and in particular, an expression library), nucleotide sequences that have been prepared by introducing mutations into a naturally occurring nucleotide sequence (using any suitable technique known per se, such as mismatch PCR), nucleotide sequence that have been prepared by PCR using overlapping primers, or nucleotide sequences that have been prepared using techniques for DNA synthesis known per se.

Nucleic acids according to the present technology can be prepared or obtained in a manner known per se, and/or can be isolated from a suitable natural source. Nucleotide sequences encoding naturally occurring (poly)peptides can for example be subjected to site-directed mutagenesis, to provide a nucleic acid molecule encoding polypeptide with sequence variation. Also, as will be clear to the skilled person, to prepare a nucleic acid, also several nucleotide sequences, such as at least one nucleotide sequence encoding a targeting moiety and for example nucleic acids encoding one or more linkers can be linked together in a suitable manner.

Techniques for generating nucleic acids will be clear to the skilled person and may for instance include, but are not limited to, automated DNA synthesis; site-directed mutagenesis; combining two or more naturally occurring and/or synthetic sequences (or two or more parts thereof), introduction of mutations that lead to the expression of a truncated expression product; introduction of one or more restriction sites (e.g., to create cassettes and/or regions that may easily be digested and/or ligated using suitable restriction enzymes), and/or the introduction of mutations by means of a PCR reaction using one or more “mismatched” primers.

In some embodiments, the nucleic acid sequence of the polynucleotide is sequence optimized for expression of the polypeptide in a suitable host.

In some embodiments, a polynucleotide of the present technology may be further optimized for in vitro or in vivo administration (e.g. for therapeutic applications).

Also provided is a vector comprising the nucleic acid molecule encoding a polypeptide of the present technology. A vector as used herein is a vehicle suitable for carrying genetic material into a cell. A vector includes naked nucleic acids, such as plasmids or mRNAs, or nucleic acids embedded into a bigger structure, such as liposomes or viral vectors.

Vectors generally comprise at least one nucleic acid that is optionally linked to one or more regulatory elements, such as for example one or more suitable promoter(s), enhancer(s), terminator(s), etc. The vector may be an expression vector, i.e., a vector suitable for expressing an encoded polypeptide or construct under suitable conditions, e.g., when the vector is introduced into a (e.g., human) cell. For DNA-based vectors, this usually includes the presence of elements for transcription (e.g., a promoter and a polyA signal) and translation (e.g., Kozak sequence).

In the vector, said at least one nucleic acid and said regulatory elements may be “operably linked” to each other, by which is generally meant that they are in a functional relationship with each other. For instance, a promoter is considered “operably linked” to a coding sequence if said promoter is able to initiate or otherwise control/regulate the transcription and/or the expression of a coding sequence (in which said coding sequence should be understood as being “under the control of” said promotor). Generally, when two nucleotide sequences are operably linked, they will be in the same orientation and usually also in the same reading frame. They will usually also be essentially contiguous, although this may also not be required.

Regulatory elements of the vector may be selected such that they are capable of providing their intended biological function in the intended host cell or host organism.

For instance, a promoter, enhancer or terminator should be “operable” in the intended host cell or host organism, by which is meant that for example said promoter should be capable of initiating or otherwise controlling/regulating the transcription and/or the expression of a nucleotide sequence—e.g., a coding sequence—to which it is operably linked.

A polynucleotide or a vector comprising the polynucleotide may be used to transform/transfect a host cell or host organism, e.g., for expression and/or production of a polypeptide. Suitable hosts or host cells for production purposes will be clear to the skilled person, and may for example be any suitable fungal, prokaryotic or eukaryotic cell or cell line or any suitable fungal, prokaryotic or eukaryotic organism. A host or host cell comprising a nucleic acid encoding a polypeptide of the present technology is also encompassed herein.

The terms “host cell” or “host organism” (jointly referred to as “host”) as used herein refers to a suitable cell or organism wherein a fully functional form of a desired polypeptide can be expressed. In the production method described herein, any host (organism) or host cell can be used provided that they are suitable for the production of an ISVD containing polypeptide. In some embodiments, the host is a non-human host.

Specific examples of suitable hosts comprise prokaryotic organisms, such as coryneform bacteria or enterobacteriaceae. Also comprised are insect cells, in particular insect cells suitable for baculovirus mediated recombinant expression like Trioplusiani or Spodoptera frugiperda derived cells, including, but not limited to BTI-TN-5B1-4 High Five™ insect cells (Invitrogen), SF9 or Sf21 cells; mammalian cells such as CHO cells and lower eukaryotic hosts comprising yeasts such as Pichia (Komagataella), Hansenula, Saccharomyces, Kluyveromyces, Candida, Torulopsis, Torulaspora, Schizosaccharomyces, Citeromyces, Pachysolen, Debaromyces, Metschunikowia, Rhodosporidium, Leucosporidium, Botryoascus, Sporidiobolus, Endomycopsis. In some embodiments, yeast is used as the host, such as e.g., Pichia pastoris. In some embodiments, the host is Pichia pastoris.

The host used in the production method will be capable of producing an ISVD containing polypeptide. It will typically be genetically modified to comprise one or more nucleic acid sequences encoding one or more ISVD containing polypeptides. Non-limiting examples of genetic modifications comprise the transformation e.g., with a plasmid or vector, or the transduction with a viral vector. Some hosts can be genetically modified by fusion techniques. Genetic modifications include the introduction of separate nucleic acid molecules into a host, e.g., plasmids or vectors, as well as direct modifications of the genetic material of the host, e.g., by integration into a chromosome of the host, e.g., by homologous recombination. Oftentimes a combination of both will occur, e.g., a host is transformed with a plasmid, which, upon homologous recombination will (at least partly) integrate into the host chromosome. The skilled person knows suitable methods of genetic modification of the host to enable the host to produce ISVD containing polypeptide.

### 5.5 Production and Purification

The terms “express”, “expression” or “expressing” as used herein in the context of a polypeptide, refers to the way in which polypeptides are synthesized, modified and regulated in a host. Traditional strategies for recombinant protein expression involve transfecting cells with a vector (e.g., DNA) that contains the template (e.g., the polynucleotide encoding the ISVD containing polypeptide) and then culturing the cells so that they transcribe and translate the desired polypeptide.

Specific conditions and genetic constructs for the expression of nucleic acids and for the production of polypeptides are described in the art, for example the general culturing methods, plasmids, promoters and leader sequences described in WO 94/25591, Gasser et al. 2006 (Biotechnol. Bioeng. 94: 535), Gasser et al. 2007 (Appl. Environ. Microbiol. 73: 6499), or Damasceno et al. 2007 (Microbiol. Biotechnol. 74: 381).

In some embodiments, a method for producing a polypeptide as described herein (e.g., a sequence having a sequence identity of more than 90% with the amino acid sequences of SEQ ID NO: 82) comprises steps of expressing the polypeptide in a suitable host cell or host organism (e.g., yeast Pichia pastoris) or in another suitable expression system; and isolating and/or purifying the polypeptide. A polypeptide of the present technology may thereby be effectively produced (e.g. has a high expression yield in microbial hosts).

In some embodiments, the titer after expression of the polypeptide as described herein is higher than 6.0 g/L cell free medium. In some embodiments, the titer after expression of the polypeptide as described herein is higher than 4.0 g/L in cell broth. In some embodiments, the titer after expression of the polypeptide as described herein is higher compared to the titer after expression of A041600087 (SEQ ID NO: 83). In some embodiments, the titer after expression of the polypeptide as described herein is higher compared to the titer after expression of A041600085 (SEQ ID NO: 137).

The terms “isolate”, “isolation”, or “isolating” as used herein in the context of a polypeptide, means that the desired polypeptide product is set apart or separated from a composition comprising cellular components and the desired polypeptide product. For instance, host cells may be lysed to extract the expressed desired polypeptide for subsequent purification.

The terms “purify”, “purification”, or “purifying” as used herein in the context of a polypeptide, refers to a series of steps to free the desired polypeptide from a complex mixture of components (e.g., components derived from cells, tissues, organisms). The purification process may separate the protein and non-protein parts of the mixture, and finally separate the desired protein product (e.g., the ISVD containing polypeptide) from all other proteins. Separation steps exploit differences in protein size, physico-chemical properties, binding affinity and biological activity. For example, (size exclusion) chromatography may be used to separate protein in solution.

The term “concentrate”, “concentration”, or “concentrating” as used herein in the context of a protein, refers to increasing the amount of protein in an aqueous sample, e.g., for storage or for biopharmaceutical applications. Methods are available to the skilled person to provide information on how to analyze protein concentration using, e.g., UV protein spectroscopy measurements, traditional dye-based absorbance measurements, BCA, Lowry and Bradford assays, the fluorescent dye-based assays, amine derivatization and detergent partition assays.

The first step of an ISVD polypeptide purification process is often referred to as “the capture step”. The purpose of the capture step is to have a first reduction of process-related impurities (for example, but not limited to, host cell proteins (HCPs), color and DNA) and to capture the ISVD polypeptide product while maintaining a high recovery. In some embodiments, the capture step refers to the first purification step on protein A chromatography in bind and elute mode.

The second step of a purification process is often referred to as “the polish step” which aims at purity improvement. For instance, as the second purification step of an ISVD polypeptide purification process, an ion exchange chromatography step in bind and elute mode can be used to remove/reduce product related variants (e.g., but not limited to, High-molecular Weight (HMW) species, Low-Molecular Weight (LMW) species, and other charged variants) as well as some process related impurities (e.g., but not limited to, HCP, residual Protein A, DNA) still present after the capture step.

After the polish step, an ultrafiltration/diafiltration/ultrafiltration (UF/DF/UF) step may be added to concentrate the polypeptide and exchange buffers.

In some embodiments, a polypeptide of the present technology has improved product quality (e.g. reduced amount of low molecular weight species and/or reduced amount of high molecular high species and/or aggregation) after production and further purification. In some embodiments, a polypeptide of the present technology has improved long-term stability under storage conditions (e.g., as measured by turbidity or opalescence of concentrations of 100 mg/mL of polypeptide).

The term “biophysical property” of a polypeptide refers to the physical properties of a biological process including but not limited to melting temperature (Tm), aggregation temperature (Tagg), amount of high molecular weight species (HMW), the amount of low molecular weight species (LMW), turbidity and/or opalescence, and particle formation. In one aspect, the measurement (e.g., by turbidity/opalescence, reverse phase chromatography, capillary gel electrophoresis, size exclusion high performance liquid chromatography) of a biophysical property of “a polypeptide” refers to the measurement of the biophysical property of “a population of polypeptides”.

The terms “stability” and “stable” as used herein in the context of a polypeptide, refer to the resistance of the polypeptide to aggregation, to the formation of degradation products and/or to the formation of fragmentation products under given transportation and/or storage conditions. Apart from this and/or in addition, the “stable” polypeptide retains biological activity under given transportation and/or storage conditions. The stability of said polypeptide can be assessed by degrees of aggregation, degradation and/or fragmentation (as measured e.g. by SE-HPLC, RP-(U)HPLC, IEX-HPLC, subvisible particle counting, analytical ultracentrifugation, dynamic light scattering, OD320/OD280 ratio measurement, OD500 measurement, elastic light scattering, etc.), and/or by % of biological activity (as measured e.g. by ELISA, Biacore, etc.) compared to a reference polypeptide. For example, a reference polypeptide may be a reference standard frozen at −20° C. or below −60° C. (such as e.g. −80° C.) consisting of the same polypeptide at the same concentration and in the same buffer as the stressed samples but without applying the stress conditions, which reference formulation regularly gives a main peak by SE-HPLC, RP-(U)HPLC and/or IEX-HPLC and/or keeps its biological activity in Biacore and/or ELISA.

The term “long-term stability under storage conditions” or “stability under storage conditions” as used herein in the context of a polypeptide, refers to the stability of the polypeptide during transportation and/or during storage at −20° C., below −60° C., 5° C. for 4 weeks, 3 months, 6 months, 1 year, 2 years, 3 years or longer.

The term “long-term stability under accelerated or stressed conditions” as used herein in the context of a polypeptide, refers to the stability of the polypeptide during transportation and/or during storage at 25° C. and 40° C. for 2 weeks, 4 weeks, 3 months, 6 months or longer.

The term “turbidity” or “opalescence” as used herein refers to a cloudiness or haziness of a fluid caused by large numbers of individual particles and/or intermolecular attraction of the molecules that are generally invisible to the naked eye. For example, turbidity or opalescence may be measured at concentrations of 100 mg/mL of polypeptide. Turbidity and opalescence can be measured using OD500 and opalescence is visually compared to opalescence standards. Turbidity can be measured by a nephelometer (e.g., as provided by BMG, Labtech, Thermofischer).

The term “low molecular weight species” or “LMW” as used herein in the context of a polypeptide, refers to fragments of the polypeptide. For example, LMW species may be measured by pre-peaks on reverse phase chromatography (e.g., RP-UPLC) or by capillary gel electrophoresis (CGE).

The term “high molecular weight species”, “high molecular weight variants” or “HMW” as used herein in the context of a polypeptide, refers to aggregates of the polypeptide with an apparent molecular weight equal or higher than the apparent molecular weight observed in size exclusion high performance liquid chromatography (SE-HPLC) analysis for dimers of the polypeptide (such as e.g., 90 kDa as observed for A041600035 SE-HPLC) in comparison with molecular weight markers. For example, HMW species may be measured by size exclusion high performance liquid chromatography (SE-HPLC).

The term “thermostability” as used herein in the context of a polypeptide, refers to the capacity of the polypeptide to resist irreversible change in its chemical or physical structure, often by resisting decomposition or polymerization, at a high relative temperature.

The term “aggregation” as used herein in the context of a polypeptide, refers to the development of high molecular weight aggregates, i.e. aggregates with an apparent molecular weight equal or higher than the apparent molecular weight observed in SE-HPLC analysis for dimers of the polypeptide (such as e.g., 90 kDa as observed for A041600035 SE-HPLC) in comparison with molecular weight markers. Aggregation can be assessed by various methods known in the art. Without being limiting, examples include high performance size exclusion chromatography (SE-HPLC), subvisible particle counting, analytical ultracentrifugation (AUC), dynamic light scattering (DLS), static light scattering (SLS), elastic light scattering, OD320/OD280 measurement, OD500 measurement, nephelometry.

In some embodiments, the amount of HMW species (as measured e.g., by SE-HPLC) after expression of the polypeptide as described herein is below 5%. In some embodiments, the amount of HMW species (as measured e.g., by SE-HPLC) after expression of the polypeptide as described herein is lower compared to the amount of HMW species after expression of A041600087 (SEQ ID NO: 83). In some embodiments, the amount of HMW species (as measured e.g., by SE-HPLC) after expression of the polypeptide as described herein is lower compared to the amount of HMW species after expression of A041600085 (SEQ ID NO: 137).

In some embodiments, the amount of HMW species (as measured e.g., by SE-HPLC) of the polypeptide as described herein after the first purification step on protein A chromatography is below 5%. In some embodiments, the amount of HMW species (as measured e.g., by SE-HPLC) of the polypeptide as described herein after the first purification step on protein A chromatography is lower compared to the amount of HMW species of A041600087 (SEQ ID NO: 83) after the first purification step on protein A chromatography. In some embodiments, the amount of HMW species (as measured e.g., by SE-HPLC) of the polypeptide as described herein after the first purification step on protein A chromatography is lower compared to the amount of HMW species of A041600085 (SEQ ID NO: 137) after the first purification step on protein A chromatography. In some embodiments, the amount of HMW species (as measured e.g., by SE-HPLC) of the polypeptide as described herein after the polish step (e.g., ion exchange chromatography step) is below 1%.

In some embodiments, for the polypeptide as described herein, the amount of variants with missing disulfide bridges (as measured e.g., by RP-UHPLC) at the end of the expression step is below 5%. In some embodiments, the amount of variants with missing disulfide bridges (as measured e.g., by RP-UHPLC) at the end of the expression step is lower for the polypeptide as described herein compared to the amount of variants with missing disulfide bridges at the end of the expression step for A041600087 (SEQ ID NO: 83). In some embodiments, the amount of variants with missing disulfide bridges (as measured e.g., by RP-UHPLC) at the end of the expression step is lower for the polypeptide as described herein compared to the amount of variants with missing disulfide bridges at the end of the expression step for A041600085 (SEQ ID NO: 137). In one aspect, “the amount of variants with missing disulfide bridges” refers to the measurement of a subpopulation of polypeptides variants with missing disulfide bridges as compared to the total population of polypeptides.

In some embodiments, for the polypeptide as described herein, the amount of variants with missing disulfide bridges after Cu treatment (as measured e.g., by RP-UHPLC) at the end of the expression step is below 5%. In some embodiments, the amount of variants with missing disulfide bridges after Cu treatment (as measured e.g., by RP-UHPLC) at the end of the expression step is lower for the polypeptide as described herein compared to the amount of variants with missing disulfide bridges at the end of the expression step for A041600087 (SEQ ID NO: 83). In some embodiments, the amount of variants with missing disulfide bridges after Cu treatment (as measured e.g. by RP-UHPLC) at the end of the expression step is lower for the polypeptide as described herein compared to the amount of variants with missing disulfide bridges at the end of the expression step for A041600085 (SEQ ID NO: 137).

In some embodiments, the sum of % hexose (as determined by MS-ID) on the polypeptide as described herein after the expression step is below 15%. In some embodiments, the sum of % hexose (as determined by MS-ID) on the polypeptide as described herein after the expression step is lower compared to the sum of % hexose on A041600087 (SEQ ID NO: 83) after the expression step. In some embodiments, the sum of % hexose (as determined by MS-ID) on the polypeptide as described herein after the expression step is lower compared to the sum of % hexose on A041600085 (SEQ ID NO: 137) after the expression step.

In some embodiments, the amount of LMW species (as measured e.g., by CGE) after the expression of the polypeptide as described herein is below 5%. In some embodiments, the amount of LMW species (as measured e.g., by CGE) after expression of the polypeptide as described herein is lower compared to the amount of LMW species after expression of A041600087 (SEQ ID NO: 83). In some embodiments, the amount of LMW species (as measured e.g., by CGE) after expression of the polypeptide as described herein is lower compared to the amount of LMW species after expression of A041600085 (SEQ ID NO: 137).

In some embodiments, the amount of LMW species (as measured e.g., by CGE) of the polypeptide as described herein after the first purification step on protein A chromatography is below 5%. In some embodiments, the amount of LMW species (as measured e.g., by CGE) of the polypeptide as described herein after the first purification step on protein A chromatography is lower compared to the amount of LMW species of A041600087 (SEQ ID NO: 83) after the first purification step on protein A chromatography. In some embodiments, the amount of LMW species (as measured e.g., by CGE) of the polypeptide as described herein after the first purification step on protein A chromatography is lower compared to the amount of LMW species of A041600085 (SEQ ID NO: 137) after the first purification step on protein A chromatography. In some embodiments, the amount of LMW species (as measured e.g., by CGE) of the polypeptide as described herein after the polish step (e.g., ion exchange chromatography step) is below 1%.

In some embodiments, the downstream (DS) capture recovery of the polypeptide as described herein is higher than 90%. In some embodiments, the DS capture recovery is higher for the polypeptide as described herein compared to the DS capture recovery of A041600087 (SEQ ID NO: 83). In some embodiments, the DS capture recovery is higher for the polypeptide as described herein compared to the DS capture recovery of A041600085 (SEQ ID NO: 137).

In some embodiments, the downstream (DS) capture binding by the polypeptide as described herein is higher than 18 mg/ml. In some embodiments, the DS capture binding is higher by the polypeptide as described herein compared to the DS capture binding by A041600087 (SEQ ID NO: 83). In some embodiments, the DS capture binding is higher by the polypeptide as described herein compared to the DS capture binding by A041600085 (SEQ ID NO: 137).

In some embodiments, the downstream (DS) post capture pH adjustment recovery of the polypeptide as described herein is higher than 90%. In some embodiments, the DS post capture pH adjustment recovery is higher for the polypeptide as described herein compared to the DS capture pH adjustment recovery of A041600087 (SEQ ID NO: 83). In some embodiments, the DS post capture pH adjustment recovery is higher for the polypeptide as described herein compared to the DS capture pH adjustment recovery of A041600085 (SEQ ID NO: 137).

In some embodiments, the downstream UF/DF/UF recovery of the polypeptide as described herein is higher than 90%. In some embodiments, the DS UF/DF/UF recovery is higher for the polypeptide as described herein compared to the DS UF/DF/UF recovery of A041600087 (SEQ ID NO: 83). In some embodiments, the DS UF/DF/UF recovery is higher for the polypeptide as described herein compared to the DS UF/DF/UF recovery of A041600085 (SEQ ID NO: 137).

In some embodiments the turbidity (as measured by OD500) after the polish step (e.g., ion exchange chromatography step) was lower for the polypeptide as described herein compared to the turbidity for A041600087 (SEQ ID NO: 83). In some embodiments the turbidity (as measured by OD500) was lower for the polypeptide as described herein compared to the turbidity for A041600085 (SEQ ID NO: 137).

In some embodiments, for the protein as described herein the amount of host cell proteins (HCP) (as measured by immune-enzymatic assay) after the polish step (e.g., ion exchange chromatography step) was below 30 ppm. In some embodiments, the amount of host cell proteins (HCP) (as measured by immune-enzymatic assay) was lower for the polypeptide as described herein compared to the amount of host cell proteins (HCP) for A041600087 (SEQ ID NO: 83). In some embodiments, the amount of host cell proteins (HCP) (as measured by immune-enzymatic assay) was lower for the polypeptide as described herein compared to the amount of host cell proteins (HCP) for A041600085 (SEQ ID NO: 137).

### 5.6 Compositions

The present technology also provides a composition comprising at least one polypeptide of the present technology, at least one polynucleotide encoding the polypeptide of the present technology or at least one vector comprising such a polynucleotide. The composition may be a pharmaceutical composition. The composition may further comprise at least one pharmaceutically acceptable carrier, diluent or excipient and/or adjuvant, and optionally comprise one or more further pharmaceutically active polypeptides and/or compounds.

As such, for pharmaceutical use, the polypeptides of the present technology may be formulated as a pharmaceutical preparation comprising (i) at least one polypeptide of the present technology and (ii) at least one pharmaceutically acceptable carrier, diluent, excipient, adjuvant, and/or stabilizer, and (iii) optionally one or more further pharmaceutically active polypeptides and/or compounds. Thus, according to a further aspect, the present technology relates to a pharmaceutical composition or preparation that contains at least one polypeptide of the present technology and at least one pharmaceutically acceptable carrier, diluent, excipient, adjuvant and/or stabilizer, and optionally one or more further pharmaceutically active substances.

In all cases, the ultimate dosage form must be sterile, fluid and stable under the conditions of manufacture and storage. Sterile injectable solutions are prepared by incorporating the active compound in the required amount in the appropriate solvent with various of the other ingredients enumerated above, as required, followed by filter sterilization.

### 5.7 Use of the Polypeptide

The polypeptide may be further optimized for in vitro or in vivo administration (e.g., for therapeutic applications) which may be assessed by the modulation of the activity of CX3CR1 in cells, tissues, organs or organisms. For example, the polypeptide may be for use in inhibiting the binding of CX3CR1 to fractalkine in a mammalian cell.

In one aspect, the present technology provides the polypeptide as described herein or a pharmaceutical composition comprising said polypeptide for use as a medicament. For example, a therapeutically effective amount of the polypeptide may be used in treating a disease or disorder associated with a fractalkine receptor CX3CR1 (or a single nucleotide polymorphism (SNP) variant of CX3CR1).

The terms “therapeutic agent”, “pharmaceutical agent”, “drug” refer to a compound (e.g., an ISVD containing polypeptide) used for the prevention or the treatment of a disease or disorder or for improving the well-being of a subject.

The term “disease” or “disorder” refers to changes in cells, tissues, organs or organisms as compared to normal (healthy) cells, tissues, organs or organisms. In some instances, the physiological functions related to natural organ function, homeostasis, aging, or regeneration may be changed, like abnormal organ development, inflammatory diseases, autoimmune diseases, chronic diseases, infectious disease or in cancer.

In one aspect, a disease or disorder is associated with a fractalkine receptor CX3CR1 (or a single nucleotide polymorphism (SNP) variant of CX3CR1) in cells, tissues, organs or organisms, e.g., changes in amount or activity of CX3CR1. For example, the disease, disorder or condition is selected from an inflammatory disease, cardio- and cerebrovascular atherosclerotic disorder, peripheral artery disease, myocardial infarction, restenosis, diabetic nephropathy, glomerulonephritis, human crescentic glomerulonephritis, IgA nephropathy, membranous nephropathy, lupus nephritis, vasculitis including Henoch-Schonlein purpura and Wegener's granulomatosis, rheumatoid arthritis, graft versus host disease, atopic dermatitis, inflammatory bowel disease, Crohn's disease, osteoarthritis, allograft rejection, systemic sclerosis, neurodegenerative disorder and demyelinating disease, multiple sclerosis (MS), Alzheimer's disease, pulmonary diseases such as COPD, asthma, neuropathic pain, inflammatory pain, and cancer, including ovarian cancer.

The term “subject” as used herein can be any animal, such as a mammal. Among mammals, a distinction can be made between humans and non-human mammals. Non-human mammals may be for example companion animals (e.g. dogs, cats), livestock (e.g. bovine, equine, ovine, caprine, or porcine animals), or mammals used generally for research purposes and/or for producing antibodies (e.g. mice, rats, rabbits, cats, dogs, goats, sheep, horses, pigs, non-human primates, such as cynomolgus monkeys, or camelids, such as llama or alpaca). In the context of prophylactic and/or therapeutic purposes, the subject can be any animal, and more specifically any mammal, such as, for example, a human subject. As will be clear to the skilled person, the subject to be treated will in particular be a person suffering from, or at risk from, the diseases, disorders or conditions mentioned herein.

The term “administered” as used herein refers to giving a substance (e.g., a composition comprising the polypeptide) to a subject for diagnosis, treatment or prevention of a disease. Methods of administering include parenteral administration (for example intravenous (IV), intraperitoneal, subcutaneous, intramuscular, intraluminal, intra-arterial or intrathecal administration) or oral administration. In some cases, the route of administration may be different or the same for administration of a polypeptide, polynucleotide or composition. When a polypeptide, polynucleotide or composition is intended for administration to a subject (for example, for prophylactic, therapeutic and/or diagnostic purposes), it may comprise an immunoglobulin sequence that does not occur naturally in said subject.

One or more doses can be administered. If more than one dose is administered, the doses can be administered in suitable intervals in order to maximize the effect of the polypeptide, composition, nucleic acid molecule or vector.

The term “(pharmaceutically or therapeutically or prophylactically) effective amount or dose” of a substance or composition as used herein refers to a quantity sufficient to achieve a desired therapeutic and/or prophylactic effect.

The polypeptides or polynucleotides of the present technology may be used for the prevention, treatment, alleviation and/or diagnosis of CX3CR1-associated diseases, disorders or conditions, in particular inflammatory disease, cardio- and cerebrovascular atherosclerotic disorder, peripheral artery disease, myocardial infarction, restenosis, diabetic nephropathy, glomerulonephritis, human crescentic glomerulonephritis, IgA nephropathy, membranous nephropathy, lupus nephritis, vasculitis including Henoch-Schonlein purpura and Wegener's granulomatosis, rheumatoid arthritis, graft versus host disease, atopic dermatitis, inflammatory bowel disease, Crohn's disease, osteoarthritis, allograft rejection, systemic sclerosis, neurodegenerative disorder and demyelinating disease, multiple sclerosis (MS), Alzheimer's disease, pulmonary diseases such as COPD, asthma, neuroinflammation, neuropathic pain, inflammatory pain, or cancer, including ovarian cancer.

In another aspect, the present technology provides the polypeptide as described herein or a pharmaceutical composition comprising said polypeptide for use in the treatment or prophylaxis of atherosclerosis.

In another aspect, the present technology provides the polypeptide as described herein or a pharmaceutical composition comprising said polypeptide for use in the treatment or prophylaxis of atherosclerosis by preventing and/or reducing the formation of new atherosclerotic lesions or plaques and/or by preventing or slowing progression of existing lesions and plaques.

In another aspect, the present technology provides the polypeptide as described herein or a pharmaceutical composition comprising said polypeptide for use in the treatment or prophylaxis of atherosclerosis by changing the composition of the plaques to reduce the risk of plaque rupture and atherothrombotic events.

In one aspect, the present technology provides a method of treating, or reducing the risk of inflammatory disease, cardio- and cerebrovascular atherosclerotic disorder, peripheral artery disease, myocardial infarction, restenosis, diabetic nephropathy, glomerulonephritis, human crescentic glomerulonephritis, IgA nephropathy, membranous nephropathy, lupus nephritis, vasculitis including Henoch-Schonlein purpura and Wegener's granulomatosis, rheumatoid arthritis, graft versus host disease, atopic dermatitis, inflammatory bowel disease, Crohn's disease, osteoarthritis, allograft rejection, systemic sclerosis, neurodegenerative disorder and demyelinating disease, multiple sclerosis (MS), Alzheimer's disease, pulmonary diseases such as COPD, asthma, neuroinflammation, neuropathic pain, inflammatory pain, or cancer, including ovarian cancer, in a person suffering from or at risk of, said disease or condition, wherein the method comprises administering to the person a therapeutically effective amount of a polypeptide as described herein or a pharmaceutical composition comprising said polypeptide.

In one aspect, the present technology also provides a method of treating, or reducing the risk of atherosclerosis in a person suffering from or at risk of said disease or condition, wherein the method comprises administering to the person a therapeutically effective amount of polypeptide as described herein or a pharmaceutical composition comprising said polypeptide.

In one aspect, the present technology provides a method of treating, or reducing the risk of atherosclerosis by preventing and/or reducing the formation of new atherosclerotic lesions or plaques and/or by preventing or slowing progression of existing lesions and plaques in a person suffering from or at risk of said disease or condition, wherein the method comprises administering to the person a therapeutically effective amount of polypeptide as described herein or a pharmaceutical composition comprising said polypeptide.

In one aspect, the present technology also provides a method of treating, or reducing the risk of atherosclerosis by changing the composition of the plaques so as to reduce the risk of plaque rupture and atherothrombotic events in a person suffering from or at risk of said disease or condition, wherein the method comprises administering to the person a therapeutically effective amount of a polypeptide as described herein or a pharmaceutical composition comprising said polypeptide.

In one aspect, a polypeptide as described herein is indicated for use in the treatment or prophylaxis of diseases or conditions in which modulation of activity at the CX3CR1 receptor is desirable. In one aspect, the present technology also provides a method of treating or reducing the risk of diseases or conditions in which antagonism of the CX3CR1 receptor is beneficial which comprises administering to a person suffering from or at risk of, said disease or condition, a polypeptide as described herein.

Prophylaxis is expected to be particularly relevant to the treatment of persons who have suffered a previous episode of or are otherwise considered to be at increased risk of the disease or condition in question. Persons at risk of developing a particular disease or condition generally include those having a family history of the disease or condition, or those who have been identified by genetic testing or screening to be particularly susceptible to developing the disease or condition.

It will also be clear to the skilled person that the above methods of treatment of a disease include the preparation of a medicament for the treatment of said disease.

Furthermore, it is clear that the polypeptides of the present technology can be used as an active ingredient in a medicament or pharmaceutical composition intended for the treatment of the above diseases. Thus, the present technology also relates to the use of a polypeptide of the present technology in the preparation of a pharmaceutical composition for the prevention, treatment and/or alleviation of any of the diseases, disorders or conditions mentioned hereinabove. The present technology further relates to a polypeptide of the present technology for therapeutic or prophylactic use and, specifically, for the prevention, treatment and/or alleviation of any of the diseases, disorders or conditions mentioned hereinabove. The present technology further relates to a pharmaceutical composition for use in the prevention, treatment and/or alleviation of the diseases, disorders or conditions mentioned hereinabove, wherein such composition comprises at least one polypeptide of the present technology

The polypeptides of the present technology and/or the compositions comprising the same can be administered to a patient in need thereof in any suitable manner, depending on the specific pharmaceutical formulation or composition to be used.

The polypeptides as described herein and/or the compositions comprising the same are administered according to a regimen of treatment that is suitable for preventing, treating and/or alleviating the disease, disorder or condition to be prevented, treated or alleviated. The clinician will generally be able to determine a suitable treatment regimen, depending on factors such as the disease, disorder or condition to be prevented, treated or alleviated, the severity of the disease, the severity of the symptoms thereof, the specific polypeptide of the present technology to be used, the specific route of administration and pharmaceutical formulation or composition to be used, the age, gender, weight, diet, general condition of the patient, and similar factors well known to the clinician. Generally, the treatment regimen will comprise the administration of one or more polypeptides of the present technology, or of one or more compositions comprising the same, in an effective amounts or doses.

The efficacy of the polypeptides of the present technology, and of compositions comprising the same, can be tested using any suitable in vitro assay, cell-based assay, in vivo assay and/or animal model known per se, or any combination thereof, depending on the specific disease involved. Suitable assays and animal models will be clear to the skilled person.

The term “half-life” as used herein refers to the time taken for the serum concentration of the polypeptide to be reduced by 50%, in vivo, for example due to degradation of the polypeptide and/or clearance or sequestration of the polypeptide by natural mechanisms. The in vivo half-life of a polypeptide can be determined in any manner known per se, such as by pharmacokinetic analysis. The half-life can be expressed using parameters such as the t½-alpha, t½-beta and the area under the curve (AUC). Reference is made to standard handbooks, such as Kenneth, A et al 1986 (Chemical Stability of Pharmaceuticals: A Handbook for Pharmacists) and Peters et al. 1996 (Pharmacokinetic analysis: A Practical Approach). Reference is also made to M Gibaldi and Perron 1982 (Pharmacokinetics, published by Marcel Dekker, 2nd Rev. edition). The terms “increase in half-life” or “increased half-life” refer to an increase in the t½-beta, either with or without an increase in the t½-alpha and/or the AUC or both.

In some embodiments, the polypeptide of the present present technology exhibits a half-life in the subject to be treated (e.g. blood half-life) that is long enough such that consecutive treatments can be conveniently spaced apart.

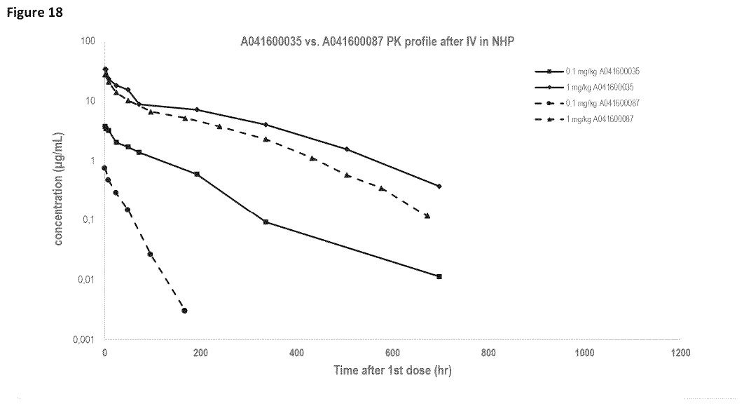

In one aspect, a polypeptide as described herein, when administered to a mammal, such as a cynomolgus monkey or a human, is characterized by a pharmacokinetics (PK) profile that differs to the PK profile observed for the polypeptide consisting of the amino acid sequence as shown in SEQ ID NO: 83 or SEQ ID NO: 137. In particular, the polypeptide may show a more prolonged serum persistence (i.e. longer half-life) as compared to the polypeptide consisting of the amino acid sequence as shown in SEQ ID NO: 83. This effect can be particularly prominent at lower doses (FIG. 18). In one aspect, the PK profile of a polypeptide as described herein does not exhibit target mediated drug deposition (TMDD). In one aspect, a polypeptide as described herein, when administered to a mammal, such as a cynomolgus monkey or a human, is characterized by a PK profile with lower target mediated drug deposition (TMDD) compared to the polypeptide consisting of the amino acid sequence as shown in SEQ ID NO: 83. Lack of TMDD provides an increased selectivity and, consequently, a better safety profile to the polypeptide described herein.

In one aspect, when the polypeptide as described herein is compared to a reference polypeptide consisting of an amino acid sequence as shown in SEQ ID NO: 83, the polypeptide has a higher exposure in a subject over a longer period after administering a similar effective dose of the polypeptide (e.g., 0.1 mg/kg or 1 mg/kg). In one aspect, the polypeptide, after administering a lower dose compared to a reference polypeptide consisting of an amino acid sequence as shown in SEQ ID NO: 83, has the same exposure compared to said reference polypeptide consisting of an amino acid sequence as shown in SEQ ID NO: 83.

According to still other embodiments, there is provided a method of diagnosing a disease, disorder or condition mediated by CX3CR1 dysfunction comprising the steps of:
a) obtaining a sample from a subject, and
b) contacting, in vitro, the sample with a polypeptide of the present technology as defined above, and
c) detecting the binding of said polypeptide to said sample, and
d) comparing the binding detected in step (c) with a standard, wherein a difference in binding relative to said sample is diagnostic of a disease, disorder or condition characterised by CX3CR1 dysfunction.

According to other embodiments, there is provided a method of diagnosing a disease, disorder or condition mediated by CX3CR1 dysfunction comprising the steps of:
a) obtaining a sample from a subject, and
b) contacting the sample with a polypeptide of the present technology as defined above;
c) determining the amount of CX3CR1 in the sample; and
d) comparing the amount determined in step (c) with a standard, wherein a difference in amount relative to said sample is diagnostic of a disease, disorder or condition characterised by CX3CR1 dysfunction.

The above diagnostic methods can also be used for monitoring the effectiveness of a therapeutic treatment of a subject.

According to other embodiments, there is provided a kit for diagnosing a disease, disorder or condition mediated by CX3CR1 dysfunction, and/or for monitoring the effectiveness of a therapeutic treatment, wherein the kit is for use in a method as defined above. Such kit may comprise at least one polypeptide, polynucleotide or composition of the present technology and, optionally, one or more media, detection means and/or in vitro or in vivo imaging agents, and, further optionally, instructions of use. Suitable in vivo imaging agents include 99mTc, 111Indium, 123Iodine, and, for magnetic resonance imaging, paramagnetic compounds.

The present technology further provides a kit comprising at least one polypeptide, polynucleotide or composition of the present technology and, additionally, one or more other components selected from the group consisting of other drugs used for the treatment of the diseases and disorders as described above, and devices as described above.

### 6. EXAMPLES

Hereinafter, the present technology is described in more detail and specifically with reference to the Examples, which however are not intended to limit the scope of the present technology.

### Example 1: Sequence Optimization of Monovalent Anti-CX3CR1 ISVDs

Anti-CX3CR1 ISVD building blocks 66B02 (SEQ ID NO: 4) and 54A12 (SEQ ID NO: 1) have been described in WO 2013/130381 (SEQ ID NOs: 1 and 2 of WO 2013/130381). These ISVD building blocks were sequence optimized into 66B02_SO (SEQ ID NO: 5) and 54A12_SO (SEQ ID NO: 2) to make them more human-like (humanization), to reduce their binding by pre-existing antibodies, to reduce antigenicity (removal of T-cell epitopes) and to remove post-translational modification (PTM) sites. Alignment of the ISVD sequences described in WO 2013/130381 and exemplary ISVDs that were sequence optimized in the present technology is given in FIG. 1.

### Example 2: Characterization of Sequence Optimized Monovalent ISVDs

The sequence optimized ISVD variants 66B02_SO (SEQ ID NO: 5) and 54A12_SO (SEQ ID NO: 2) were characterized for biophysical properties and potency.

### 2.1 Thermal Shift Assay

A thermal shift assay (TSA) was performed in a 96-well plate on a qPCR machine (LightCycler 48011, Roche). Per row, one ISVD protein was analysed in the following pH range: 4, 5, 6, 7, 8 and 9.

Per well, 5 μL of ISVD protein sample (0.8 mg/mL in D-PBS) was added to 5 μL of Sypro Orange (40× in MilliQwater; Invitrogen, Cat. No. S6551) and 10 μL of buffer (100 mM phosphate, 100 mM borate, 100 mM citrate and 115 mM NaCl with a pH ranging from 4 to 9). A temperature gradient (37 to 99° C. at a rate of 0.03° C./s) was applied, which induced unfolding of the ISVD proteins, and hence exposure of hydrophobic patches. Binding of Sypro Orange to those hydrophobic patches, caused increase in fluorescence intensity, which was measured (Ex/Em=465/580 nm). The inflection point of the first derivative of the fluorescence intensity curve at pH 7 served as a measure of the melting temperature (Tm).

The Tm (° C.) values obtained for the sequence optimized variants are given in Table B-1. All ISVDs showed acceptable Tm values.

### 2.2 Oligomerization Assay

Oligomerization propensity of monovalent ISVD proteins under stressed conditions (1 week at 45° C.) was investigated by analytical size exclusion chromatography (SE-HPLC). For this, ISVD proteins with a 3×FLAG-His6 tag, produced in E. coli, purified via IMAC followed by preparative SEC, filtered (0.22 am), at a concentration of 1 mg/mL (D PBS) were used. The SE-HPLC profiles of two 100 μL aliquots were compared: one sample was incubated for 1 week at −20° C. (TO) and the other sample for 1 week at 45° C. Samples were cleared by centrifugation for 5 minutes at 20000 RCF and subsequently analysed on a Waters Xbridge column (mobile phase 10 mM Phosphate+150 mM Arginine+10% 1-propanol pH 7, flow rate 0.5 mL/minute). The difference in relative pre-peak areas of stressed (+45° C.) and non-stressed samples (−20° C.) was calculated and reported as Δ% HMW (=% HMW 1 W 45° C.-% HMW TO).

The Δ% HMW values obtained for the sequence optimized variants are given in Table B-1. Oligomerization % was acceptable for all ISVDs.

### 2.3 Onset of Aggregation Temperature Determination (Tagg Assay)

The temperature at which an ISVD protein starts to aggregate (=temperature of onset of aggregation=Tagg) was determined by Dynamic Light Scattering (DLS) using the DynaPro Plate reader (Wyatt). For this, ISVD proteins with a 3×FLAG-His6 tag, produced in E. coli, purified via IMAC followed by preparative SEC, filtered (0.22 am), at a concentration of 1 mg/mL (D-PBS) were used. After thawing the sample was filtered over a 0.1 am membrane and centrifuged for 5 min at 14000 rpm. Samples of 30 μL (4 replicates) were heated from 40 to 80° C. at a constant rate of 0.25° C./minute with continuous recording of the light scattering intensities. The Hydrodynamic radius derived from the measured intensities was plotted against the temperature to determine the temperature at which the radius started to increase (=Tagg, ° C.).

The Tagg values obtained for the sequence optimized variants are given in Table B-1. All ISVDs had acceptable Tagg values.

### 2.4 Inhibition of CX3CL1/Fraktalkine Ligand Binding to Human and Cyno CX3CR1

The ability of purified ISVD proteins to inhibit the interaction between human CX3CL1/Fraktalkine and human and cynomolgus monkey (cyno) CX3CR1 expressed on the cell surface of CHO K1 cells was assessed with flow cytometry. CHO cells overexpressing human or cyno CX3CR1 were generated using techniques known in the art. Cells were harvested and resuspended in assay buffer (PBS, 2% FBS, 0.05% NaN3). The cells were seeded in 384-well Bio-One V-bottom plates (Greiner, Cat. No. 781280), with a total of 2E04 cells seeded per well. After washing, ISVD proteins (starting from 250 nM, 3-fold dilution, 11 points, in assay buffer) were premixed with 30 μM HSA (Sigma-Aldrich, Cat. No. A8763) and human CX3CL1 labelled with AF647 (R&D Systems, Cat. No. 365-FR; labelled in house) at a final concentration of 2E-10 M (˜EC30) and added to the cells for 4 hours at 4° C. Plates were washed 3 times and cells were resuspended in PI (Miltenyi Biotec, Cat. No. 130-093-233, 1000-fold diluted in assay buffer). Cell suspensions were analysed with iQue Screener PLUS or iQue 3(Intellicyt). The IC50s were estimated by dose response modelling. Curves were fit using 4 parameter logistic regression in GraphPad (GraphPad Software Inc.).

The IC50 values obtained for the sequence optimized variants are given in Table B-1. The sequence-optimized building blocks retained acceptable Tm values for use in further development.

TABLE B-1
Characterization of selected monovalent ISVDs.

| | | | | CX3CL1 competition FACS | |
|---|---|---|---|---|---|
| | Biophysical characterization | | | CHO K1 human | CHO K1 cyno |
| Short | Tm | Tagg | Δ % | CX3CR1 | CX3CR1 |
| description | (° C.) | (° C.) | HMW | IC50 (M) | IC50 (M) |
| 66B02_SO | 65 | 66 | 0.4 | 4.8E−10 | 2.9E−09 |
| 307 (D1E) | 69 | 67 | 0.1 | NA | NA |
| 54A12_SO | 64 | 55 | 0.2 | 8.9E−10 | 6.0E−09 |

NA: not available

### Example 3: Preparation of Multivalent ISVD Constructs

A panel of 24 formats was generated containing sequence optimized anti-CX3CR1 ISVD building blocks, i.e. 66B02_SO (SEQ ID NO: 5), 66B02_SO (E1D) (SEQ ID NO: 6), 54A12_SO (SEQ ID NO: 2) and/or 54A12_SO (E1D) (SEQ ID NO: 3), and an albumin binding building block (ALB23002; SEQ ID NO: 37). The building blocks were fused head-to-tail with 9GS (SEQ ID NO: 69), 20GS (SEQ ID NO: 73) or 35GS (SEQ ID NO: 76) linkers (Table A-7). In this panel, the ALB23002 was present in second or third position. All 24 formats also had an alanine (A) introduced at the C-terminal end. Two formats (A041600085 and A041600087) were generated with building block 307 (SEQ ID NO: 7) (building block 307 is SEQ ID NO: 223 of WO 2013/130381), 307 (DiE) (SEQ ID NO: 8), and ALB23002 (SEQ ID NO: 37) or ALB11 (SEQ ID NO: 42) respectively. In A041600085 a C-terminal alanine (A) was introduced (see Table B-2).

TABLE B-2
Overview of exemplary polypeptides against CX3CR1.

| ISVD construct | | Building blocks | | | | | |
|---|---|---|---|---|---|---|---|
| Name | ID | ISVD1 | L1 | ISVD2 | L2 | ISVD3 | A |
| A041600010 | 153 | 66B02_SO (E1D) | 9GS | ALB23002 | 9GS | 66B02_SO | A |
| A041600011 | 154 | 66B02_SO (E1D) | 9GS | 66B02_SO | 9GS | ALB23002 | A |
| A041600025 | 155 | 54A12_SO (E1D) | 9GS | ALB23002 | 9GS | 54A12_SO | A |
| A041600026 | 156 | 66B02_SO (E1D) | 20GS | ALB23002 | 20GS | 66B02_SO | A |
| A041600027 | 157 | 66B02_SO (E1D) | 9GS | ALB23002 | 20GS | 66B02_SO | A |
| A041600028 | 158 | 66B02_SO (E1D) | 9GS | ALB23002 | 35GS | 66B02_SO | A |
| A041600029 | 159 | 66B02_SO (E1D) | 20GS | ALB23002 | 9GS | 66B02_SO | A |
| A041600030 | 160 | 66B02_SO (E1D) | 20GS | ALB23002 | 35GS | 66B02_SO | A |
| A041600031 | 161 | 66B02_SO (E1D) | 9GS | ALB23002 | 9GS | 54A12_SO | A |
| A041600032 | 162 | 66B02_SO (E1D) | 35GS | ALB23002 | 9GS | 66B02_SO | A |
| A041600033 | 163 | 66B02_SO (E1D) | 35GS | ALB23002 | 20GS | 66B02_SO | A |
| A041600034 | 164 | 54A12_SO (E1D) | 9GS | ALB23002 | 9GS | 66B02_SO | A |
| A041600035 | 82 | 54A12_SO (E1D) | 9GS | 54A12_SO | 9GS | ALB23002 | A |
| A041600036 | 165 | 66B02_SO (E1D) | 20GS | 66B02_SO | 9GS | ALB23002 | A |
| A041600037 | 166 | 66B02_SO (E1D) | 20GS | 66B02_SO | 35GS | ALB23002 | A |
| A041600038 | 167 | 66B02_SO (E1D) | 35GS | 66B02_SO | 9GS | ALB23002 | A |
| A041600039 | 168 | 66B02_SO (E1D) | 35GS | 66B02_SO | 20GS | ALB23002 | A |
| A041600040 | 169 | 66B02_SO (E1D) | 9GS | 54A12_SO | 9GS | ALB23002 | A |
| A041600041 | 170 | 54A12_SO (E1D) | 9GS | 66B02_SO | 9GS | ALB23002 | A |
| A041600042 | 171 | 66B02_SO (E1D) | 20GS | 66B02_SO | 20GS | ALB23002 | A |
| A041600043 | 172 | 66B02_SO (E1D) | 35GS | 66B02_SO | 35GS | ALB23002 | A |
| A041600044 | 173 | 66B02_SO (E1D) | 9GS | 66B02_SO | 20GS | ALB23002 | A |
| A041600045 | 174 | 66B02_SO (E1D) | 9GS | 66B02_SO | 35GS | ALB23002 | A |
| A041600046 | 175 | 66B02_SO (E1D) | 35GS | ALB23002 | 35GS | 66B02_SO | A |
| A041600085 | 137 | 307 | 9GS | ALB23002 | 9GS | 307 (D1E) | A |
| A041600087 | 83 | 307 | 9GS | ALB11 | 9GS | 307 (D1E) | |

### Example 4: Characterization of the Multivalent ISVD Constructs by Expression Profiling

The 24 different multivalent ISVD constructs were characterized for their expression levels in P. pastoris (Komagataella phaffii). In short, the different ISVD formats were transformed in P. pastoris, strain NRRL Y-11430 (ATCC 76273) and plated out on YPDS plates (2% Peptone (w/v), 1% Yeast Extract (w/v), 18.2% Sorbitol (w/v), 1.5% Select agar (w/v), 2% Glucose (w/v)) containing 1000 μg/ml Zeocin®. Twenty clones per multivalent ISVD construct were picked following selection on agar and used for an expression profiling in 96-well deep-well plates. To this end, Pichia clones were inoculated in 100 μl BGCM medium (10% Citric acid 1M pH 6.0, 0.1% Glycerol (v/v), 13.4 g/L Yeast Nitrogen Base (without Amino Acids) (w/v), 0.00004% (w/v) Biotin, 2.6% Peptone (w/v), 1.4% Yeast Extract (w/v)) in a master 96-well deep-well plate. After an incubation of 24 hours (30° C., 200 rpm), 5 μl of the preculture was used to inoculate 100 μl BGCM medium in a second 96-well deep-well plate. After 24 hours of growth, the cultures in the expression plate were induced by adding 75 μl MeOH (0.66% v/v) to each well. During fermentation, another 75 μl MeOH (0.66% v/v) was added to each culture to maintain induction. After 24 h of induction, the cells were harvested, and the supernatant was analysed. The amount of expressed protein in the medium was analysed on Octet Red384 using ProtA biosensors. Following normalization to a reference, the relative normalized expression was plotted for each clone within a respective format and the average normalized expression per format was determined. The formats were then ordered according to the respective ISVD building blocks and linker lengths.

An overview of the expression profiling can be found in FIG. 2. Within each format, the expression levels of clones showed considerable variation. Based on the average normalized expression (Table B-3), a number of trends were picked up. A higher expression was obtained for formats containing the 54A12_SO building block compared to the formats with the 66B02_SO building block. Higher titers could be obtained if the ALB32002 building block was at the C-terminal position. As linker between the first and second building block, 9GS linkers were preferred over 20 GS and 35 GS. As linker between the second and third building block, the 20GS linker was preferred over the 9GS linker and 35GS linker.

TABLE B-3
Overview of normalized expression values grouped per multivalent ISVD construct type.
Group I

| | | | | | | Average Normalized |
|---|---|---|---|---|---|---|
| A0416000- | Formats | | | | | expression values |
| 31 | 66B02_SO (E1D) | 19GS | ALB23002 | 9GS | 54A12_SO | 190 |
| 40 | 66B02_SO (E1D) | 9GS | 54A12_SO | 9GS | ALB23002 | 313 |
| 34 | 54A12_SO (E1D) | 9G | ALB23002 | 9GS | 66B02_SO | 301 |
| 41 | 54A12_SO (E1D) | 9GS | 66B02_SO | 9GS | ALB23002 | 391 |

Group II

| | | | | | Average Normalized |
|---|---|---|---|---|---|
| A0416000- | Formats | | | | expression values |
| 25 | 54A12_SO (E1D) | 9GS ALB23002 | 9GS | 54A12_SO | 328 |
| 35 | 54A12_SO (E1D) | 9GS 54A12_SO | 9GS | ALB23002 | 336 |

Group III

| | | | | | | Average Normalized |
|---|---|---|---|---|---|---|
| A0416000- | Formats | | | | | expression values |
| 10 | 66B02_SO (E1D) | 9GS | ALB23002 | 9GS | 66B02_SO | 206 |
| 27 | 66B02_SO (E1D) | 9GS | ALB23002 | 20GS | 66B02_SO | 211 |
| 28 | 66B02_SO (E1D) | 9GS | ALB23002 | 35GS | 66B02_SO | 180 |
| 29 | 66B02_SO (E1D) | 20GS | ALB23002 | 9GS | 66B02_SO | 229 |
| 26 | 66B02_SO (E1D) | 20GS | ALB23002 | 20GS | 66B02_SO | 297 |
| 30 | 66B02_SO (E1D) | 20GS | ALB23002 | 35GS | 66B02_SO | 212 |
| 32 | 66B02_SO (E1D) | 35GS | ALB23002 | 9GS | 66B02_SO | 93 |
| 33 | 66B02_SO (E1D) | 35GS | ALB23002 | 20GS | 66B02_SO | 110 |
| 46 | 66B02_SO (E1D) | 35GS | ALB23002 | 35GS | 66B02_SO | 105 |

Group IV

| | | | | | | Average Normalized |
|---|---|---|---|---|---|---|
| A0416000- | Formats | | | | | expression values |
| 11 | 66B02_SO (E1D) | 9GS | 66B02_SO | 9GS | ALB23002 | 219 |
| 44 | 66B02_SO (E1D) | 9GS | 66B02_SO | 20GS | ALB23002 | 174 |
| 45 | 66B02_SO (E1D) | 9GS | 66B02_SO | 35GS | ALB23002 | 127 |
| 36 | 66B02_SO (E1D) | 20GS | 66B02_SO | 9GS | ALB23002 | 196 |
| 42 | 66B02_SO (E1D) | 20GS | 66B02_SO | 20GS | ALB23002 | 379 |
| 37 | 66B02_SO (E1D) | 20GS | 66B02_SO | 35GS | ALB23002 | 143 |
| 38 | 66B02_SO (E1D) | 35GS | 66B02_SO | 9GS | ALB23002 | 143 |
| 39 | 66B02_SO (E1D) | 35GS | 66B02_SO | 20GS | ALB23002 | 206 |
| 43 | 66B02_SO (E1D) | 35GS | 66B02_SO | 35GS | ALB23002 | 120 |

Based on the ranking obtained after expression profiling, seven formats were evaluated in fed-batch fermentation. Most of the formats expressed >2 g/L. Ten ISVD formats were selected (from the 24 formats) for further characterization (see Table B-4).

Formats A041600085 and A041600087 were not included in the expression profiling experiment but were included for further characterization.

### Example 5: In Vitro Characterization of the Multivalent ISVD Constructs

### 5.1 Characterization of the Multivalent ISVD Constructs in CX3CR1 Binding Assay

The selected ISVD formats and A041600085 were characterized for binding on CHO K1 human and cyno CX3CR1 cells with flow cytometry in the presence of 30 μM HSA and compared to A041600087 (SEQ ID NO: 83).

CHO cells overexpressing human or cynomolgus CX3CR1 were generated using techniques known in the art. Cells were harvested and resuspended in assay buffer (PBS, 2% FBS, 0.05% NaN3) and seeded separately in 384-well Bio-One V-bottom plates (Greiner, Cat. No. 781280), with a total of 2E04 cells seeded per well. After washing, serial dilutions of multivalent ISVD construct (starting from 250 nM, 3-fold dilution, 11 points, diluted in assay buffer), were added and incubated for 4 hours at 4° C. Binding was performed in the presence of 30 uM HSA (Sigma-Aldrich, Cat. No. A8763). Plates were then washed 3 times and cells were incubated with an anti-VHH mouse antibody (3300-fold diluted in assay buffer) for 30 minutes at 4′° C. Subsequently, plates were washed 3 times and cells were incubated in Goat anti-mouse Fc APC (Jackson ImmunoResearch, Cat. No. 115-135-164/115-116-071) (100-fold diluted in assay buffer) for 30 minutes at 4° C. Plates were finally washed 3 times and cells were resuspended in PI (Miltenyi Biotec, Cat. No. 130-093-233, 1000-fold diluted in assay buffer). Cell suspensions were analyzed with iQue Screener PLUS (Intellicyt). EC50s were estimated by dose response modelling. Curves were fit using 4 parameter logistic regression in GraphPad (GraphPad Software Inc.). A purified anti-human CX3CR1 antibody [K0124E1] (BioLegend, Cat. No. 355701/355702) was used as positive control to detect CX3CR1 expression on the cell surface.

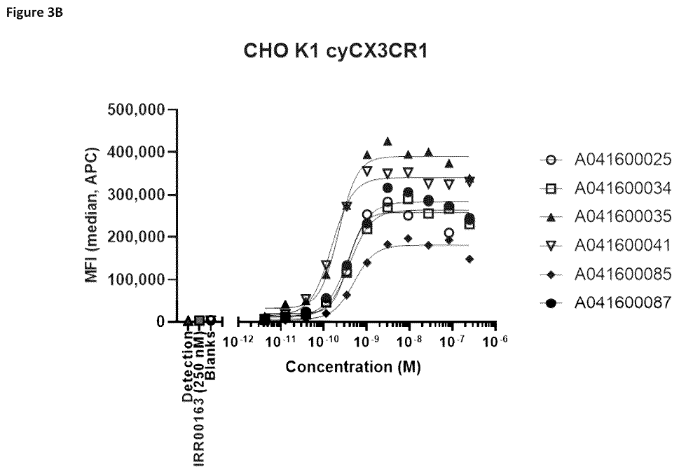

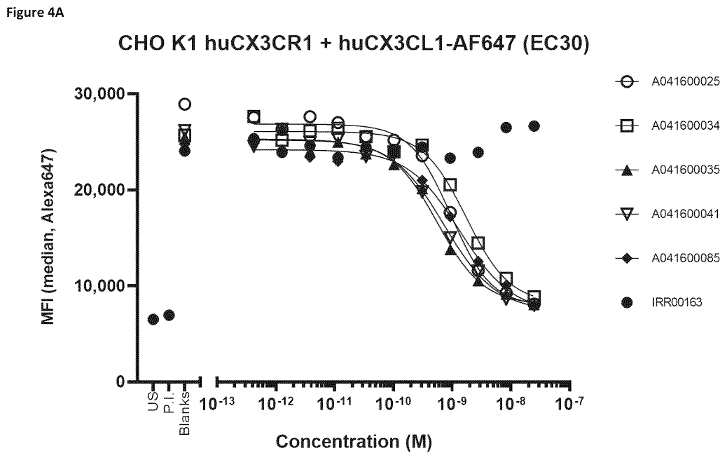

The values obtained are reported in Table B-4. All formats showed binding to human CX3CR1 with an EC50 between 0.1 and 0.6 nM and cross-reactivity with cyno CX3CR1 (maximum 3.3-fold difference in EC50). Binding curves for selected formats are shown in FIG. 3.

TABLE B-4
In vitro characterization of selected anti-CX3CR1 multivalent
ISVD constructs in CX3CR1 binding assay.

| | CX3CR1 binding | | | |
|---|---|---|---|---|
| | CHO K1 huCX3CR1 | | CHO K1 cyCX3CR1 | |
| ISVD construct | EC50 (M) | top | EC50 (M) | top |
| A041600010 | 4.1E−10 | 22787 | 4.6E−10 | 213627 |
| A041600011 | 7.9E−11 | 44155 | 1.1E−10 | 331272 |
| A041600025 | 4.6E−10 | 28710 | 3.6E−10 | 266475 |
| A041600026 | 2.9E−10 | 35606 | 2.5E−10 | 289907 |
| A041600031 | 3.0E−10 | 31329 | 3.2E−10 | 300213 |
| A041600034 | 6.3E−10 | 28217 | 4.5E−10 | 272554 |
| A041600035 | 3.6E−10 | 50938 | 2.4E−10 | 410595 |
| A041600040 | 1.0E−10 | 42224 | 1.4E−10 | 363461 |
| A041600041 | 2.2E−10 | 41839 | 1.5E−10 | 346220 |
| A041600042 | 1.3E−10 | 40048 | 1.2E−10 | 372556 |
| A041600085 | 1.7E−10 | 14571 | 5.6E−10 | 185555 |
| A041600087 | 1.6E−10 | 31331 | 3.8E−10 | 290845 |

### 5.2 Characterization of the Multivalent ISVD Constructs in a CX3CL1/Fractalkine Ligand Competition Assay

The 10 selected multivalent ISVD constructs, formats A041600085 and A041600087 and “KAND-567” were further characterized in a competition assay with AF-647 labelled soluble CX3CL1/Fraktalkine ligand at an EC30 concentration in the presence of 30 μl M HSA as described in Example 2.4.

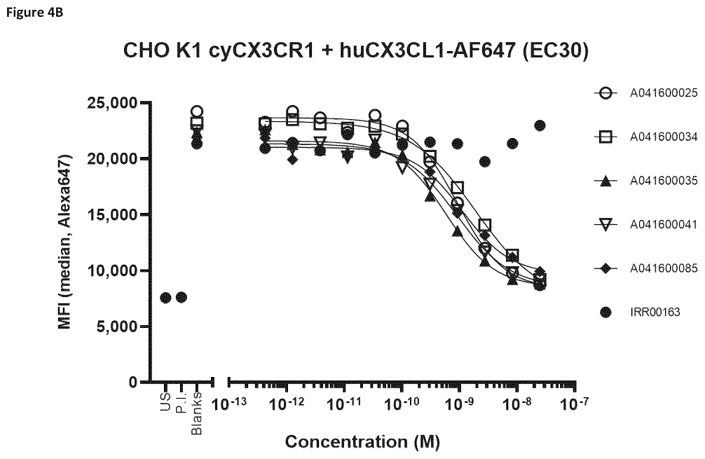

The values obtained are reported in Table B-5. All formats showed quite similar IC50s which range from 0.7 to 7.7 nM with a maximum of 8-fold difference on cyno CX3CR1. Competition curves for 5 selected multivalent ISVD constructs are shown in FIG. 4.

TABLE B-5
In vitro characterization of selected anti-CX3CR1 multivalent ISVD
constructs in a CX3CL1/Fractalkine ligand competition assay.

| | Soluble huCX3CL1-AF647 competition | | | |
|---|---|---|---|---|
| | CHO K1 huCX3CR1 | | CHO K1 cyCX3CR1 | |
| ISVD | IC50 | % | IC50 | % |
| construct | (M) | inhibition | (M) | inhibition |
| A041600010 | 3.8E−09 | 94 | 2.4E−09 | 113 |
| A041600011 | 6.6E−10 | 94 | 1.2E−09 | 111 |
| A041600025 | 3.8E−09 | 98 | 1.7E−09 | 104 |
| A041600026 | 1.1E−09 | 102 | 1.2E−09 | 104 |
| A041600031 | 2.1E−09 | 96 | 1.2E−09 | 105 |
| A041600034 | 4.5E−09 | 95 | 1.8E−09 | 104 |
| A041600035 | 2.5E−09 | 99 | 1.1E−09 | 105 |
| A041600040 | 9.9E−10 | 101 | 6.2E−10 | 117 |
| A041600041 | 2.7E−09 | 97 | 1.8E−09 | 107 |
| A041600042 | 1.4E−09 | 95 | 6.7E−10 | 104 |
| A041600085 | 7.7E−09 | 103 | 1.1E−09 | 102 |
| A041600087 | 1.5E−09 | 103 | 7.6E−10 | 104 |
| KAND-567 | ~1 μM | NA | NA | NA |

NA: not available

### 5.3 Characterization of the Multivalent ISVD Constructs for Inhibition of CX3CL1/Fraktalkine Induced Chemotaxis of BA/F3 Cells Expressing Human CX3CR1

The 10 selected multivalent ISVD constructs, formats A041600085 and A041600087, and “KAND-567” were also tested in a chemotaxis assay in which Ba/F3-huCX3CR1 cells were stimulated with soluble CX3CL1/Fraktalkine in the presence of 30 μM HSA. Ba/F3 cells overexpressing human CX3CR1 were generated using techniques known in the art. The ability of purified ISVD constructs to inhibit the CX3CL1-induced migration of BA/F3 cells expressing human CX3CR1 was assessed in a Boyden chamber-based method using NeuroProbe Chemotax plates (Cat. No. 106-5). In short, the bottom well of the chamber was filled with 0.5, 1 or 5 nM human CX3CL1 (R&D Systems, Cat. No. 365-FR) in 30 μL assay buffer (RPMI medium containing 30 μM HSA (Sigma, Cat. No. A8763)). The BA/F3 cells were pre-incubated with the ISVD construct or inhibitor for 30 min at 37° C. in RPMI medium containing 30 μM HSA (Sigma, Cat. No. SLCD9951). Next, 50 μL of the ISVD construct or inhibitor/cell suspension (containing 1E05 cells) mix was added on the membrane and the plates were incubated for 3 h at 37° C. with 5% CO2. The migrated cells were quantified using CellTiter Glo where the ATP content of a cell is measured. For this, 30 μL of the remaining cell suspension was transferred to a white Costar 96-well plate (#3917) and the wells were washed with 20 μL assay medium. Next, 50 μL of CellTiter Glo reagent (Promega, Cat. No. G7571) was added to each well and the contents were mixed for 5 min at 1000 RPM on an orbital shaker to induce cell lysis. After 10 min of incubation at RT, the signal was measured with Envision 2 (RLU-1 sec).

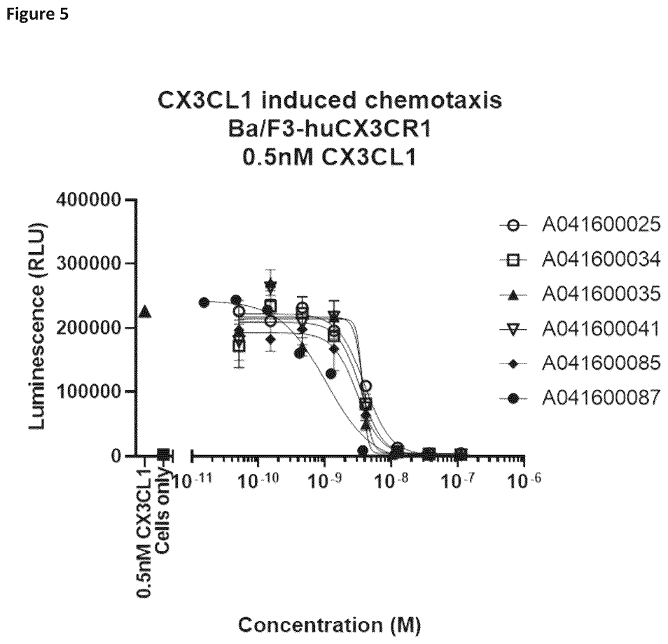

All tested multivalent ISVD constructs potently blocked huCX3CL1-induced chemotaxis of Ba/F3-huCX3CR1 cells with an IC50 of 1.6 to 3.6 nM (when 1 nM of CX3CL1 was used). The assay was performed multiple times, and the geometric mean of the different IC50s is present in Table B-6. A graph of the chemotaxis inhibition of 5 multivalent ISVD constructs that were selected for further characterization is shown in FIG. 5 and compared to A041600087.

TABLE B-6
In vitro characterization of selected anti-CX3CR1
multivalent ISVD constructs in chemotaxis assay.

| | Chemotaxis on Ba/F3-huCX3CR1 induced with soluble huCX3CL1 | | | |
|---|---|---|---|---|
| | 0.5 nM CX3CL1 | | 1 nM CX3CL1 | |
| ISVD construct | IC50 (M) | N= | IC50 (M) | N= |
| A041600010 | 2.7E−09 | 3 | 3.0E−09 | 5 |
| A041600011 | NA | | 2.7E−09 | 3 |
| A041600025 | 3.8E−09 | 7 | 3.4E−09 | 8 |
| A041600026 | 2.0E−09 | 2 | 2.6E−09 | 3 |
| A041600031 | 2.1E−09 | 2 | 2.5E−09 | 3 |
| A041600034 | 3.7E−09 | 5 | 3.6E−09 | 4 |
| A041600035 | 3.8E−09 | 4 | 2.8E−09 | 4 |
| A041600040 | 3.0E−09 | 3 | 2.0E−09 | 3 |
| A041600041 | 2.7E−09 | 5 | 2.9E−09 | 3 |
| A041600042 | 3.1E−09 | 2 | 2.4E−09 | 3 |
| A041600085 | 2.6E−09 | 4 | 3.1E−09 | 7 |
| A041600087 | 1.1E−09 | 12 | 1.6E−09 | 15 |
| KAND-567 | NA | | No clear inhibition | |

NA: not available

### Example 6: In Vitro Characterization of Selected Multivalent ISVD Constructs

Based on potency, high titer, biophysical properties and building block diversity, the multivalent ISVD constructs A041600025, A041600034, A041600041, A041600035 and A041600085 were selected for further characterization.

### 6.1 Characterization of the Multivalent ISVD Constructs for Binding Different huCX3CR1 Variants

Two important SNPs are described in huCX3CR1, i.e. V2491 (Rs3732379) and T280M (Rs3732378), which might influence receptor activity and binding by the ISVD. Both SNPs are prevalent in the global population. Different combinations of these SNPs were introduced in human CX3CR1, and binding by 5 selected multivalent ISVD constructs was assessed with flow cytometry after transient transfection of the huCX3CR1 variants in HEK293T cells.

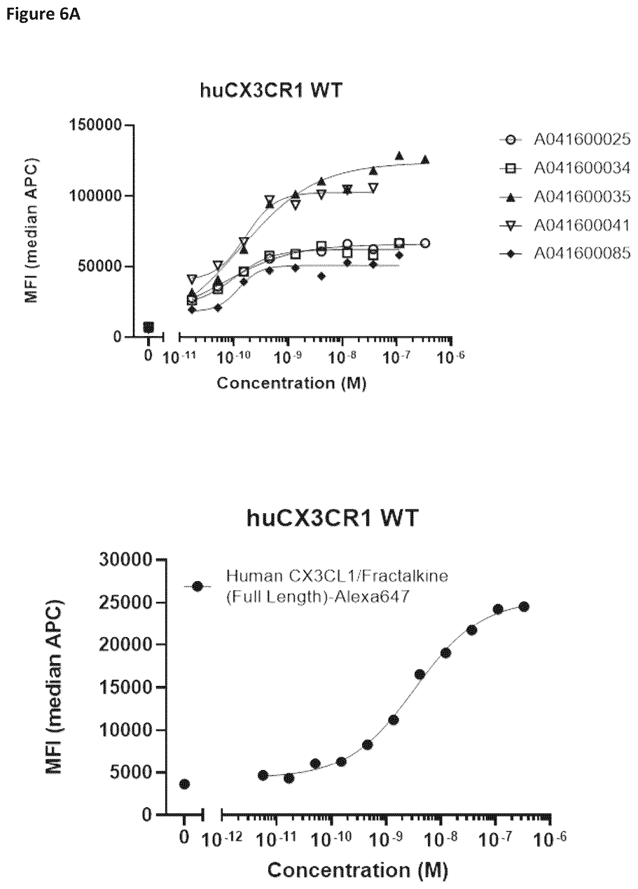

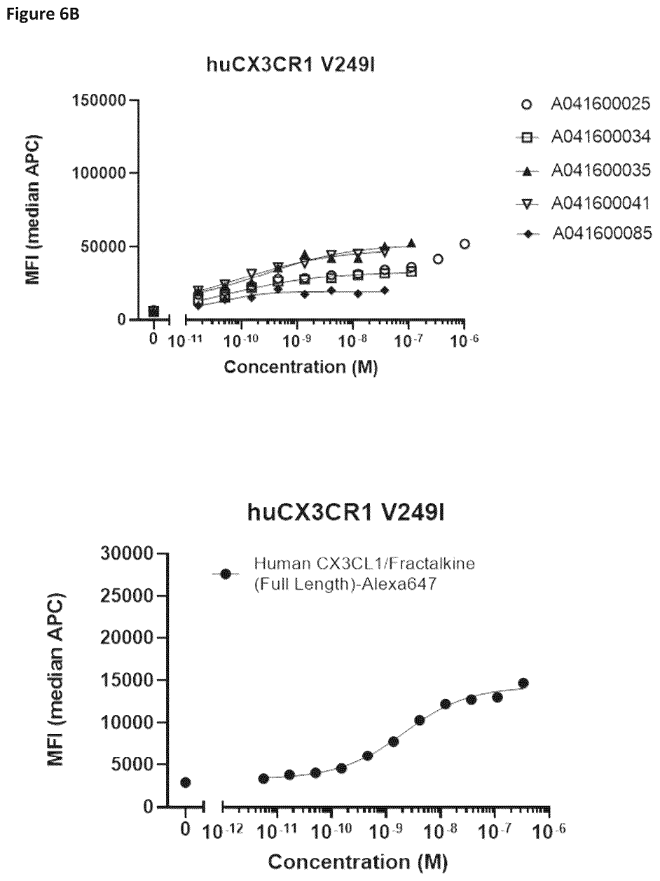

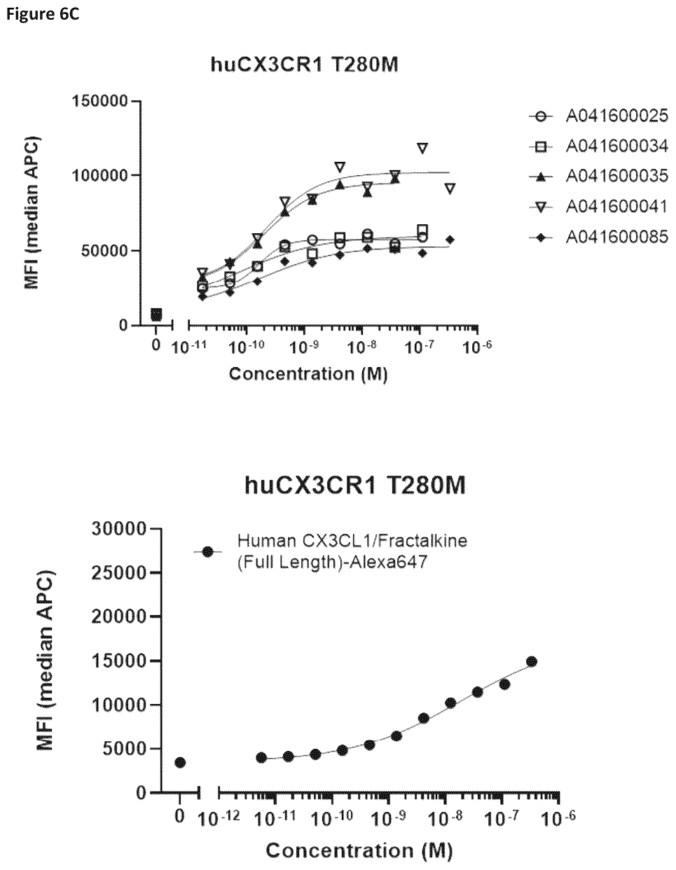

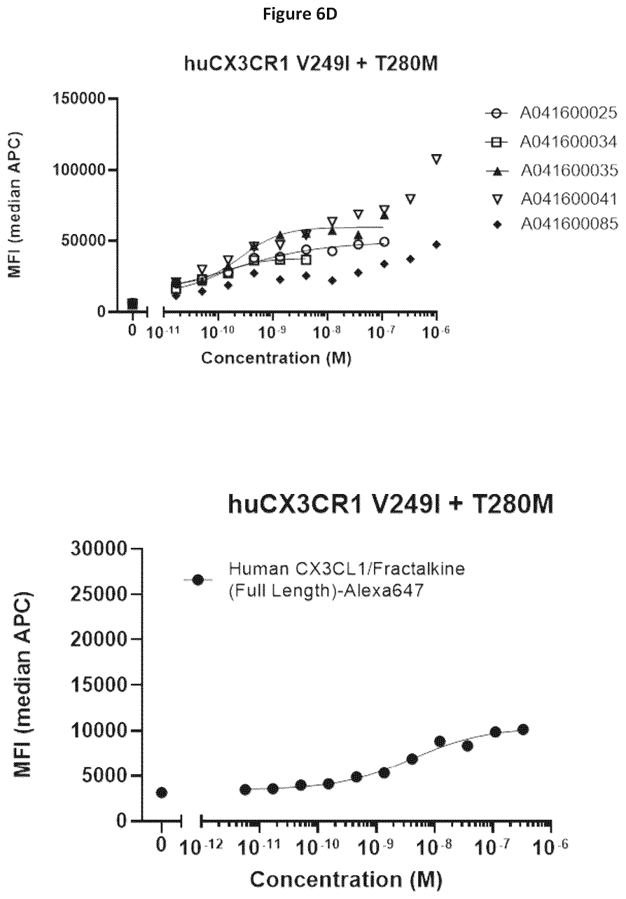

In short, the CX3CR1 constructs were cloned into pcDNA3.1 (ThermoFisher Scientific, Cat. No. V79020) and plasmid DNA was prepared from Escherichia coli TOP10 cells. HEK293T cells were seeded at a concentration of 1.5E06 cells per T75 flask and incubated overnight at 37° C. in DMEM medium (Gibco, Cat. No. 31966) supplemented with 10% FBS (Sigma. Cat. No. F7524). The medium was then replaced by Opti-MEM medium (Gibco, Cat. No. 31985). A mixture of 9 μg plasmid DNA, 27 μL Fugene 6 (Promega, Cat. No. E2691) in a final volume of 1 mL Opti-MEM was incubated for 15 minutes at room temperature and then added to the cells. After 3 hours incubation at 37° C., 10 mL of DMEM supplemented with 20% FBS was added, and incubation continued. After 48 hours, cells were washed with PBS and suspended with 4 mL trypsin EDTA (Gibco, Cat. No. 25200-056) followed by addition of 6 mL DMEM medium supplemented with 10% FBS. The binding assay was performed as described in Example 5.1. In addition to binding of the ISVDs, also binding of AF-647 labelled soluble CX3CL1/Fraktalkine ligand to the different receptor variants was included as positive control for CX3CR1 expression. The tested multivalent ISVD constructs showed similar binding to the different huCX3CR1 variants, and no binding to the parental HEK293T cells (Table B-7 and FIG. 6).

TABLE B-7
In vitro characterization of selected multivalent ISVD constructs
in CX3CR1 binding assay with different CX3CR1 variants.

| | HEK293T: human CX3CR1 | | | |
|---|---|---|---|---|
| ISVD construct | WT | V249I | T280M | V249I/T280M |
| A041600025 | 6.5E−11 | no fit | 1.6E−10 | 1.7E−10 |
| A041600034 | 1.0E−10 | 4.5E−11 | 7.4E−11 | 9.0E−11 |
| A041600035 | 1.3E−10 | 2.5E−10 | 2.0E−10 | 2.7E−10 |
| A041600041 | 1.6E−10 | 8.9E−11 | 2.2E−10 | no fit |
| A041600085 | 1.2E−10 | 5.7E−11 | 1.7E−10 | no fit |
| huCX3CL1 | 3.3E−09 | 2.1E−09 | 1.9E−08 | 4.2E−09 |

### 6.2 Binding by Pre-Existing Antibodies

The 5 selected multivalent ISVD constructs were tested for binding by pre-existing antibodies present in 96 serum samples from healthy volunteers using the ProteOn XPR36 (Bio-Rad Laboratories, Inc.) and compared to A041600087.

The results show that binding by pre-existing antibodies is very low for the 5 multivalent ISVD constructs.

### 6.3 Specificity of the Multivalent ISVD Constructs

### 6.3.1 Specificity Towards CCR2 and CCR5

Specificity of the 5 selected multivalent ISVD constructs for the huCX3CR1 receptor was evaluated by performing a FACS binding experiment on CHO-K1 cells expressing human CCR2 or human CCR5. CHO cells overexpressing huCCR2 or huCCR5 were generated using techniques known in the art. Binding of the multivalent ISVD constructs to huCR2 or huCCR5 expressed on the cell surfaces was examined by flow cytometry as described in example 5.1.

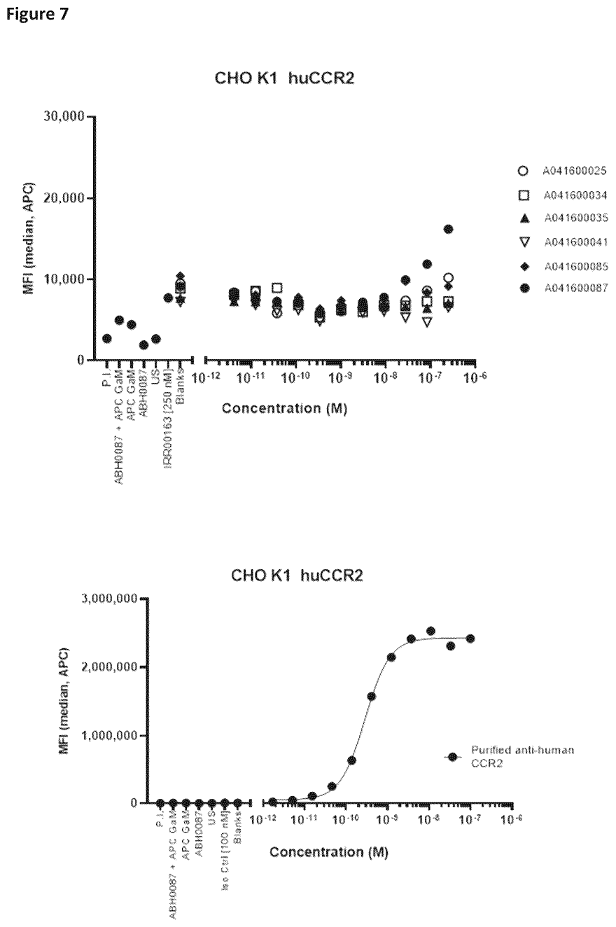

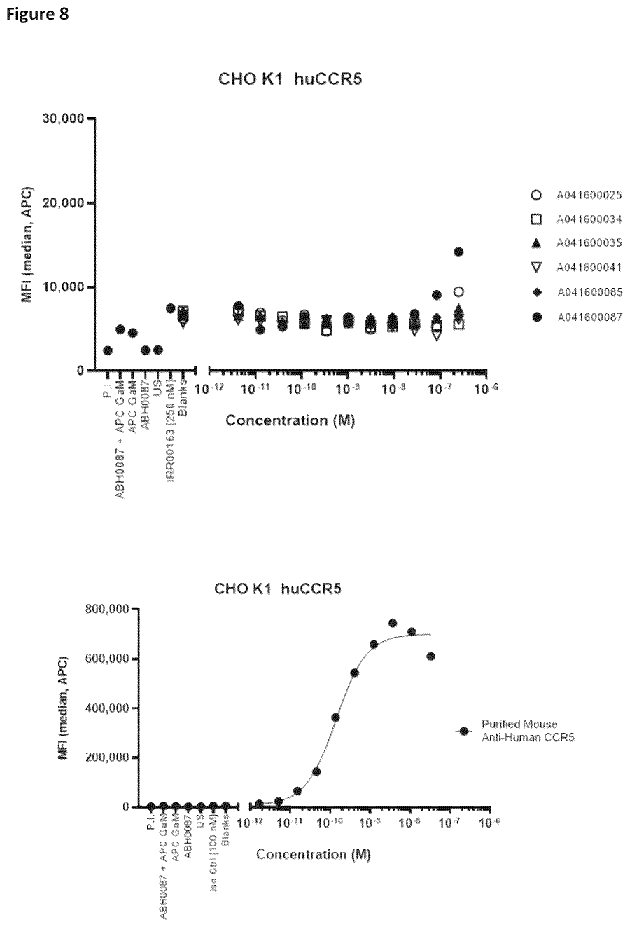

No binding to huCCR2 or huCCR5 could be detected. Binding curves are shown in FIGS. 7 and 8. A purified anti-human CCR2 [K036C2] (BioLegend, Cat. No. 357201/357202) or anti-human CCR5 [3A9](BD Biosciences Cat. No. 556041) was used as positive control to detect CCR2 or CCR5 expression on the cell surface.

### 6.3.2 Specificity in Human Membrane Protein Array

Binding of A041600035 and A041600087 was evaluated in a human membrane protein array (MPA) to profile specificity and target selectivity.

The MPA is a protein library composed of 6,000 distinct human membrane protein clones, each overexpressed in live cells from expression plasmids. Each clone was individually transfected in separate wells of a 384-well plate followed by a 36 h incubation (Tucker et al. 2018, Proc. Natl. Acad. Sci. USA. 29: 115(22): E4990-9). Cells expressing each individual MPA protein clone were arrayed in duplicate in a matrix format for high-throughput screening.

Before screening on the MPA, the optimal A041600035 and A041600087 concentration of 20 μg/mL for screening was determined on HEK-293T cells expressing positive (CX3CR1 or membrane-tethered Protein A) and negative (mock-transfected) binding controls followed by detection by flow cytometry using a fluorescently labeled secondary antibody that binds ISVDs.

A041600035 and A041600087 ligand were added to the MPA at the predetermined concentration of 20 ug/mL, and binding across the protein library was measured on an Intellicyt iQue using the fluorescently labeled secondary antibody on unfixed cells. Each array plate contains both positive (Fc-binding) and negative (empty vector) controls to ensure plate-by-plate reproducibility. Non-specific fluorescence was determined to be any value below 3 standard deviations of the mean background value.

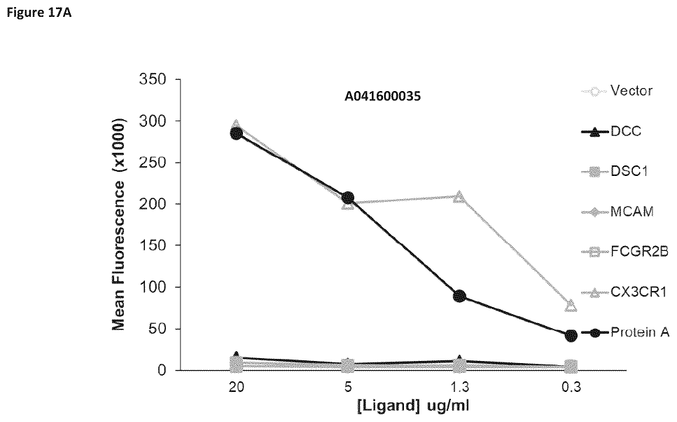

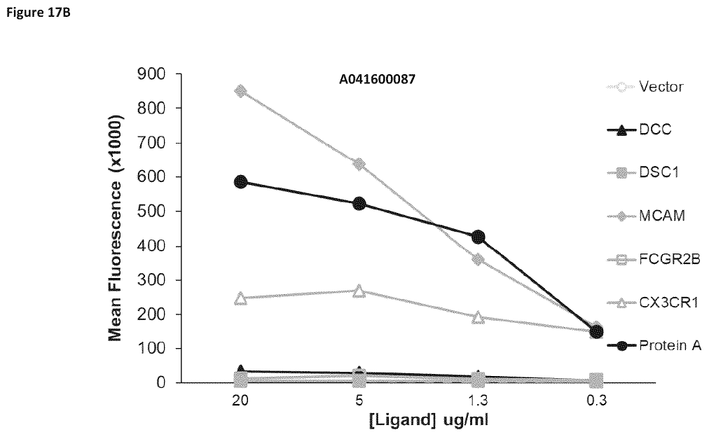

Test ligand interactions with any targets identified by MPA screening were confirmed in a second flow cytometry experiment using serial dilutions of the test antibody in buffer containing 10% human serum albumin, and the target identity was re-verified by sequencing. Validated targets demonstrating a dose response and MFI ≥2-fold above background at the two highest concentrations tested are shown in FIG. 17A for A041600035 and in FIG. 17B for A041600087.

Specific binding to CX3CR1 but to no other human membrane protein was observed for A041600035. Specific binding to CX3CR1 and MCAM (alternative name: MUC18; https://www.uniprot.org/uniprot/P43121) was observed for A041600087.

### Example 7: Manufacturability Assessment of Selected Multivalent ISVD Constructs

Based on potency, high titer, biophysical properties and building block diversity, the multivalent ISVD constructs A041600085, A041600025, A041600034, A041600041, and A041600035 were selected for further manufacturability assessment.

Manufacturability experiments focused on product titer and purity during upstream processing (USP), as well as on purification recovery, purity and DSP platform fit after downstream processing (DSP).

### 7.1. Product Titer and Purity During Upstream Processing (USP)

The titer in the supernatant and cell broth at the end of the fermentation was measured.

The other criteria measured were related to product purity and were assessed using different methods. ISVD-related HMW-species at the end of the fermentation were quantified as the sum of the relative areas of the pre-peaks in the SE-HPLC chromatogram after a Protein A clean-up of the fermentation samples. Less than 5% HMW-species is preferred at the start of process development.

The post-peak area of the RP-UHPLC chromatogram mainly gives information about the amount of variants with missing disulfide bridges. These variants cannot be removed by the purification process (although some spontaneous oxidation has been observed) and should therefore be kept to a minimum. Other variants such as carbamylated variants or, exceptionally, low molecular weight variants are also observed as post-peaks, but identification requires additional characterization (e.g., LC-MS). Hence, the post-peak area is considered as only indicative for variants with missing disulfide bridges. Preferably, the amount of variants with missing disulfide bridges after copper-treatment (WO 2010/125187) is less than 5%.

The banding pattern on SDS-PAGE has no numerical scores or threshold. The main band should be clearly visible at the expected molecular weight and there should be no or only a limited amount of degradation observed on the SDS-PAGE gel. The degree of O-glycosylation of the formats was determined by MS-ID. Ideally the sum of % hexose is below 15%.

The USP manufacturability of the exemplary multivalent ISVD constructs is shown in Table B-8. A041600025 showed a very good titer and product quality at the end of fermentation. For most USP manufacturability criteria, high quality scores were obtained, except for the LMW-species and the percentage of post-peaks on RP-UHPLC before copper treatment (just above the threshold).

A041600034 showed a lower titer and poor product quality at the end of fermentation. A high percentage of LMW-species was observed and the percentage of post-peaks on RP-UHPLC before and after copper treatment (aCu) was just above the threshold. The lower titer can have a negative impact on the development timelines, fermentation volumes and cost of goods.

A041600035 had the second highest titer at the end of fermentation and the best quality. Product quality was very good except for a high percentage of LMWs.

A041600041 showed a good titer. Only one USP manufacturability criterion had a high-quality score (percentage of post-peaks on RP-UHPLC after copper treatment).

A041600085 showed a good titer. Only one USP manufacturability criterion had a high-quality score (percentage of post-peaks on RP-UHPLC after copper treatment).

TABLE B-8
Manufacturability assessment of 5 multivalent ISVD constructs from a USP perspective.

| | | | | | RPC ^{a} | | | |
|---|---|---|---|---|---|---|---|---|
| | | | | | before Cu | | | |
| | Titer ^{a, c} | Titer ^{a, b, c} | | | treatment | RPC ^{a} | | |
| | (PA-HPLC) | (PA-HPLC) | SEC ^{a} | CGE ^{a} | % Post- | % Post- | SDS- | MS-ID ^{a} |
| | (g/L | (g/L | (% HMW | (% LMW | peaks (incl. | peaks (incl. | PAGE ^{a} | (Molar % |
| Construct | cell-free) | cell-broth) | species) | species) | missing S-S) | missing S-S) | (USP) | O-hexose) |
| A041600025 | 12.8 | 9.7 | 2.0 | 8.0* | 5.1* | 3.2 | OK | 7.0 |
| A041600034 | 5.4* | 3.3 | 1.0 | 14.9** | 7.3* | 5.2* | OK | 6.5 |
| A041600035 | 19.8 | 7.1 | 2.2 | 10.8** | 14.4 | 2.2 | OK | 12.6 |
| A041600041 | 7.3 | 5.4 | 11.4** | 12.1** | 9.8* | 3.0 | OK | 14.5 |
| A041600085 | 8.7 | 6.4 | 9.3* | 17.1** | 17.6** | 3.5 | OK | 8.5 |

Asterisk labeling: no asterisk, high quality; one asterisk, medium quality; two asterisks, lower quality.
^{a} Sample aCu unless mentioned otherwise, analysis after ProtA clean-up except for PA-HPLC.
^{b} Calculated using end of fermentation wet cell weight (typically 350-400 g/L) as follows: Titer cell free [g/L] × ((1000-wet cell weight [g/L])/1000) × 1.12.
^{c} PA-HPLC titers determined with generic standard curve.

### 7.2 Downstream Processing (DSP) Manufacturability Assessment

The DSP manufacturability assessment aims to evaluate the multivalent ISVD construct purification suitability. A qualitative and quantitative assessment, including criteria for capture resin binding capacity (>18 mg/mL), capture resin recovery (>90%), eluate pH adjustment recovery (>95%), UF/DF/UF recovery (>85%) and conservation of the molecular integrity during UF/DF/UF, was performed during a larger scale purification.

Results of the DSP manufacturability assessment are summarized in Table B-9. The target capture resin binding capacity was set at ‘>18 mg/mL’ taking into account the trivalent format of the multivalent ISVD constructs. Out of the 5 multivalent ISVD constructs tested, two candidates had binding capacity to the capture resin below this pre-defined limit, A041600085 and A041600041. This lower capture resin binding capacity translated for these two multivalent ISVD constructs into lower recovery during the capture, at a loading of 20 mg of ISVD/mL of resin. Limited protein loss was measured during pH adjustment for A041600035, A041600025, A041600041, but a more significant drop was observed for A041600085, A041600034 leading to recoveries below 95%. Finally, all five multivalent ISVD constructs had high recovery during the final UF/DF/UF step with no impact on their molecular integrity.

TABLE B-9
Manufacturability assessment of 5 selected multivalent ISVD constructs in downstream processing (DSP).

| Success Criteria | A041600085 | A041600034 | A041600035 | A041600025 | A041600041 |
|---|---|---|---|---|---|
| DSP Capture recovery (%) at 20 | 78* | 96 | 92 | 93 | 81* |
| mg/ml load (PA-HPLC) | | | | | |
| DSP Capture binding capacity (mg/mL) | 15.5* | 19.3 | 18.3 | 18.7 | 16.3* |
| DSP Post-capture pH adjustment | 86* | 87* | 95 | 97 | 97 |
| recovery (%) (PA-HPLC) (based on | | | | | |
| additional pH adjustment study) | | | | | |
| DSP UF/DF/UF recovery (%) (OD280) | 95.5 | 98.1 | 95.9 | 96.6 | 101.5 |
| DSP UF/DF/UF conservation | No change | No change | No change | No change | No change |
| molecular integrity | | | | | |

Asterisk labeling: no asterisk, high quality; one asterisk, medium quality; two asterisks, lower quality.

The DSP manufacturability assessment on the 5 selected multivalent ISVD constructs confirmed that A041600035 and A041600025 met all the criteria: capture binding capacity (>18 mg/mL), capture recovery at a load of 20 mg/mL (>90%), pH adjustment recovery (>95%), UF/DF/UF recovery (>85%) and conservation of molecular integrity during UF/DF/UF (Table B-9).

### 7.3 Molecular Profiling of Exemplary Multivalent ISVD Constructs

Molecular profiling was assessed for the 5 selected multivalent ISVD constructs to identify and characterize the biochemical properties and structure-related liabilities for the five selected multivalent ISVD constructs.

### 7.3.1 Determination of High Molecular Weight Products

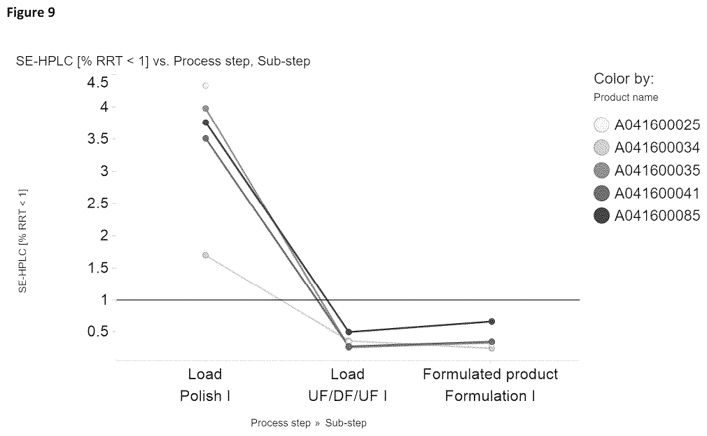

The level of high molecular weight products (HMWs) was determined by Size exclusion high performance liquid chromatography (SE-HPLC). Results are shown in FIG. 9.

The sum of HMWs after capture was lower for A041600034 (1.5% HMWs) compared to the four other candidates. However, HMWs decreased below 1% after polish chromatography for all five selected multivalent ISVD constructs. In addition, this level of HMWs remained stable below 1% during UF/DF/UF and until the final step of formulation.

### 7.3.2 Determination of Low Molecular Weight Products

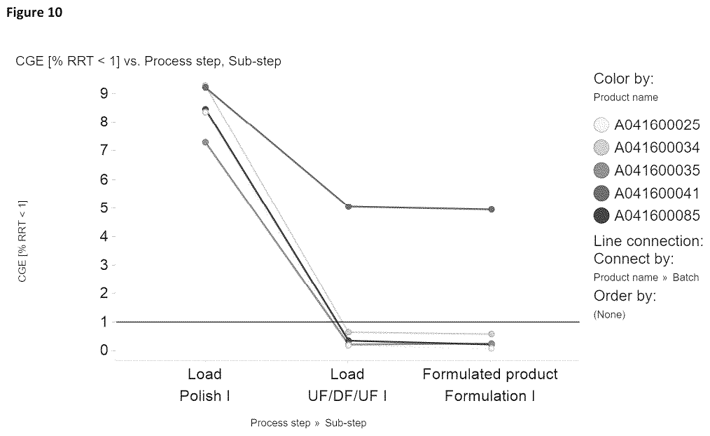

The level of low molecular weight products (LMWs) was determined by Capillary gel electrophoresis (CGE). A polish step by cation exchange chromatography (CEX) significantly reduced the lower molecular weight products (LMWs) but to different levels, as shown in FIG. 10. A041600041 did not decrease below 1% after the polish step. The level of LMWs remains stable during UF/DF/UF until the final step of formulation in all 5 multivalent ISVD constructs.

### 7.3.3 Visual Inspection—Turbidity

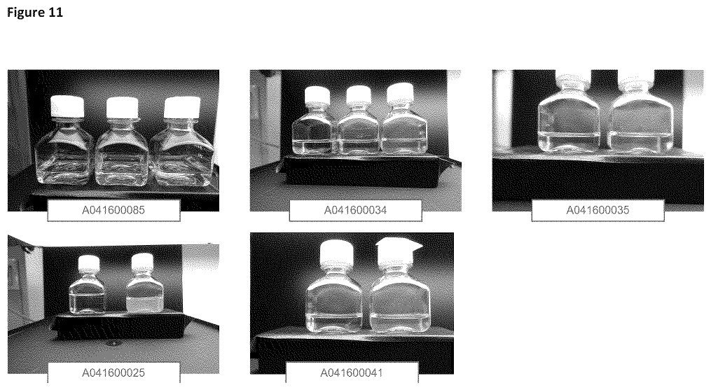

Visual inspection was done by direct observation of a large volume of the final product (FIG. 11). Results were classified as ‘Clear’, ‘Slightly Turbid’ or ‘Turbid’. At the intermediate concentration of 50 mg/mL, all multivalent ISVD constructs tested were ‘Clear’. At the target concentration of 100 mg/mL, four out of the five multivalent ISVD constructs were found ‘Slightly Turbid’, and significant turbidity and/or opalescence was observed for A041600025 and was therefore classified as ‘Turbid’.

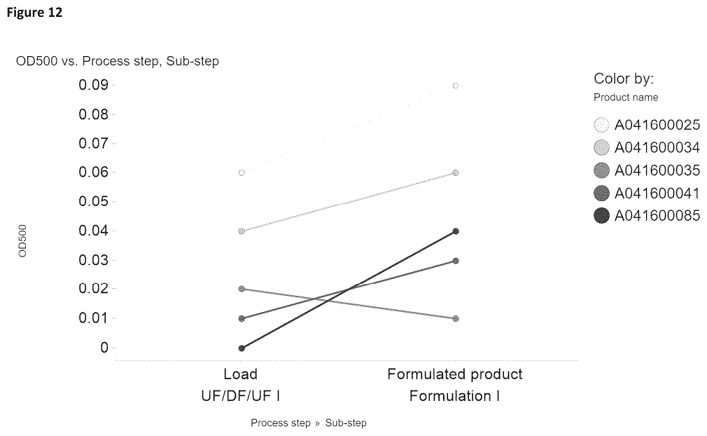

Further analysis of this turbidity and/or opalescence was done using OD500 (FIG. 12) and by comparing with a turbidity standard curve. Both confirm the direct observation of samples. A041600025 is confirmed as ‘Turbid’, whereas other multivalent ISVD constructs are deemed ‘Slightly Turbid’ at 100 mg/mL. In addition, final product of the 5 multivalent ISVD constructs was found slightly colored (yellow) and free of particles by direct visualization.

### 7.3.4 Measurement of Host Cell Proteins

The host cell proteins (HCP) were measured with an immune-enzymatic assay for the 5 multivalent ISVD constructs after the different DSP steps. The HCP content in A04160035 was below 30 ppm. The 4 other multivalent ISVD constructs had an HCP ranging from 30 to 60 ppm.

### 7.3.5 Conclusion of the Molecular Profiling

Table B-10 summarizes the outcome of the molecular profiling data.

TABLE B-10
Molecular profiling assessment of the multivalent ISVD constructs in DSP.

| | A041600085 | | A041600034 | | A041600035 | | A041600025 | | A041600041 | |
|---|---|---|---|---|---|---|---|---|---|---|
| Success | 50 | 100 | 50 | 100 | 50 | 100 | 50 | 100 | 50 | 100 |
| Criteria | mg/mL | mg/mL | mg/mL | mg/mL | mg/mL | mg/mL | mg/mL | mg/mL | mg/mL | mg/mL |
| Clarity | Clear | Slightly | Clear | Slightly | Clear | Slightly | Clear | Turbid** | Clear | Slightly |
| (Visual | | Turbid* | | Turbid* | | Turbid* | | | | Turbid* |
| inspection) | | | | | | | | | | |
| Color | Slightly | | Slightly | | Slightly | | Slightly | | Slightly | |
| (Visual | Yellow | | Yellow | | Yellow | | Yellow | | Yellow | |
| inspection) | | | | | | | | | | |
| Purity | 99.9% | 99.8% | 99.5% | 99.4% | 99.7% | 99.8% | 99.8% | 99.1% | 95.1* | 95.1%* |
| (CGE) | | | | | | | | | | |
| HMW | 0.5% | 0.7% | 0.2% | 0.3% | 0.3% | 0.3% | 0.6% | 0.2% | 0.4% | 0.4% |
| (SE-HPLC) | | | | | | | | | | |
| LMW | 0.1% | 0.2% | 0.5% | 0.6% | 0.3% | 0.2% | 0.2% | 0.1% | 4.9%** | 4.9%** |
| (CGE) | | | | | | | | | | |

| | | | | | |
|---|---|---|---|---|---|
| HCP | 30-60 ppm | 30-60 ppm | <30 ppm | 30-60 ppm | 30-60 ppm |

Asterisk labeling: no asterisk, high quality; one asterisk, medium quality; two asterisks, lower quality.

### Example 8: Further Characterization of the Selected Multivalent ISVD Constructs

### 8.1 Characterization of the Multivalent ISVD Constructs for Blocking Hu CX3CL1 Induced Phosphorylation of ERK in BA/F3 Cells Expressing Human CX3CR1

The ability of multivalent ISVD constructs, A041600034, A041600035, A041600085 and A041600087 as well as “KAND-567”, to inhibit the CX3CL1-induced ERK phosphorylation in BA/F3 cells expressing human CX3CR1, was assessed in a HTRF-based assay, using the Advanced phospho-ERK (Thr202/Tyr204) cellular kit from Cisbio Bioassays (Cat. No. 64AERPEG|64AERPEH|64AERPET). In short, the ISVD construct or inhibitor was premixed with 1.3 nM CX3CL1 (˜EC30) in assay buffer (RPMI+30 μM HSA) and 50 μL was added to each well of a Costar 96-well plate (#3596). Next, 50 μL cell suspension (6E04 BA/F3 cells in assay buffer) was added to each well and incubated for 10 min at RT. The plates were centrifuged for 2 min at 300 g and the assay medium was removed by inverting the plates. Next, 50 μL of lysis/ERK blocking solution (prepared according to the manufacturer's instructions) was added and the plates were incubated for 5 min at RT while being shaken at 900 RPM, followed by an additional incubation of 40 min at RT at the bench. Before read-out, the plates were shaken again for 30 sec at 900 RPM and 16 uL of the lysate (without pipetting up and down) was transferred to a HTRF compatible 384-well plate (Perkin Elmer; ProxiPlate-384 Plus, White 384-shallow well Microplate; #6008280). The donor and acceptor antibodies were mixed in a 1:1 ratio in detection buffer, and 4 μL of this mix was added to each well and incubated for 3 h in the dark at RT. The signal was determined with the TECAN F200, and the ratio was determined by dividing the TRF signal at 665 nm by the TRF signal at 620 nm and multiplied by 1E04.

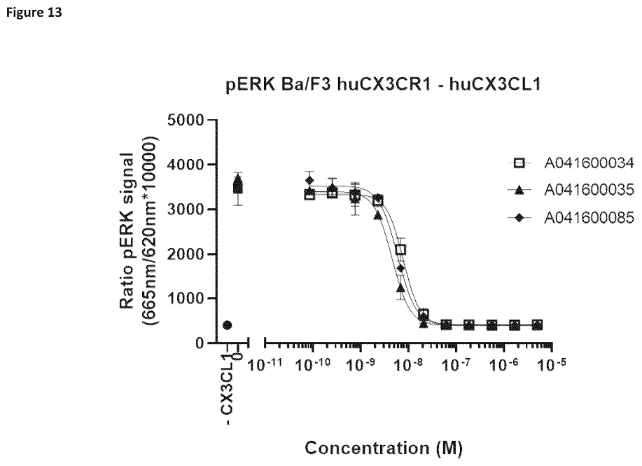

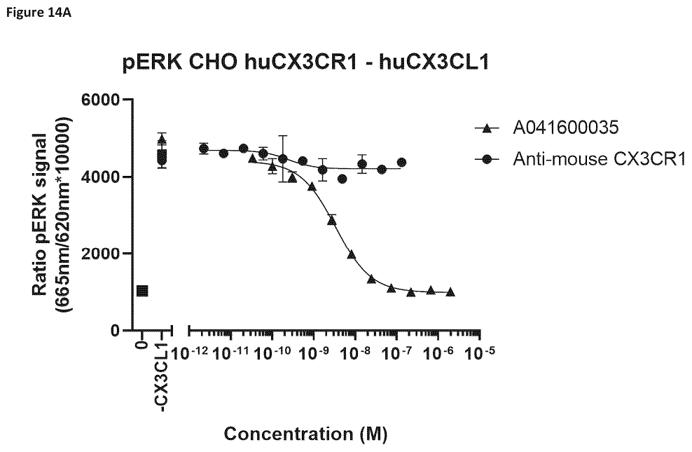

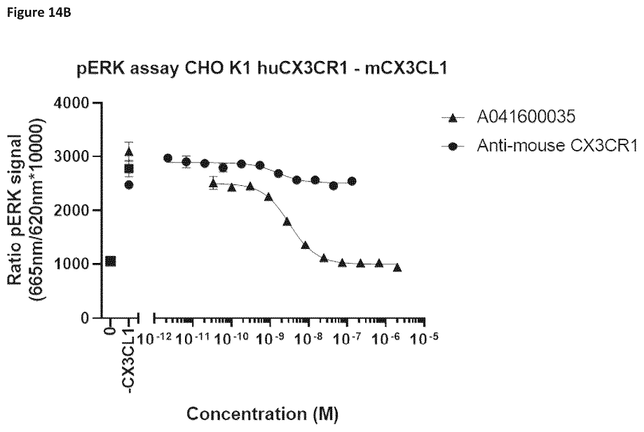

The values obtained are reported in Table B-11. The multivalent ISVD constructs block huCX3CL1 induced phosphorylation of ERK in cells expressing human CX3CR1 with an IC50 from 2.7 to 5.4 nM (see FIG. 13). The assay was performed multiple times, and the geometric mean of the different IC50s is shown in Table B-11.

TABLE B-11
In vitro characterization of selected anti-CX3CR1
multivalent ISVD constructs in pERK assay.

pERK on Ba/F3-huCX3CR1 induced
with soluble huCX3CL1

| ISVD construct | IC50 (M) | N= |
|---|---|---|
| A041600034 | 5.4E−09 | 3 |
| A041600035 | 3.0E−09 | 3 |
| A041600085 | 3.6E−09 | 2 |
| A041600087 | 2.7E−09 | 3 |
| KAND-567 | ~3 μM | |

### 8.2 Characterization of the Multivalent ISVD A041600035 for Blocking Human or Mouse CX3CL1 Induced Phosphorylation of ERK in CHO K1 Cells Expressing Human CX3CR1

The pERK assay was repeated for A041600035 with CHO K1 cells expressing human CX3CR1, stimulated with human or mouse CX3CL1. A surrogate antibody recognizing mouse CX3CR1 (AB5715, Biolegend Cat. No. 149002) was used as a positive control for the blocking of CX3CL1 induced phosphorylation of ERK in CHO K1 mouse CX3CR1 cells.

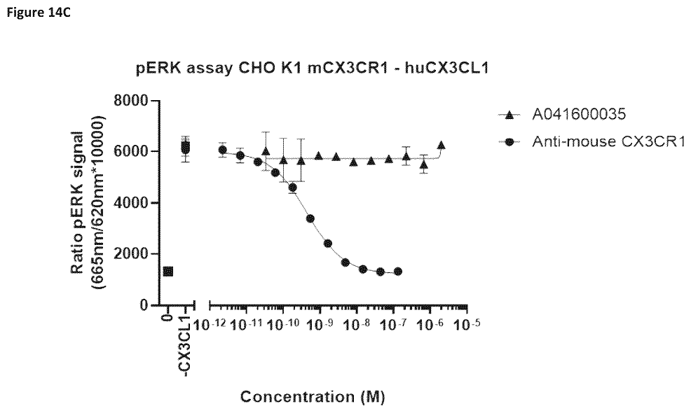

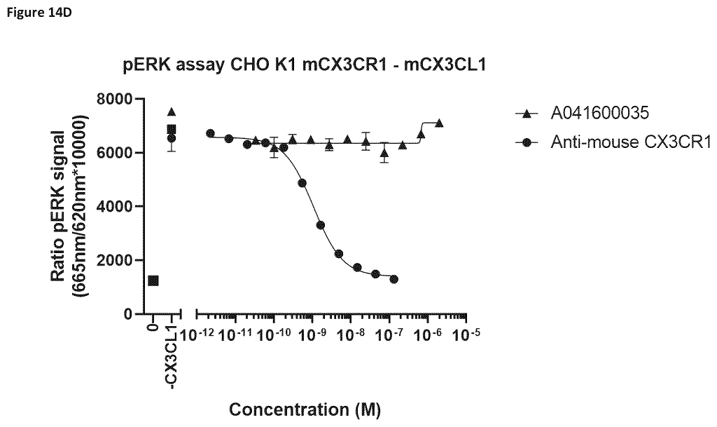

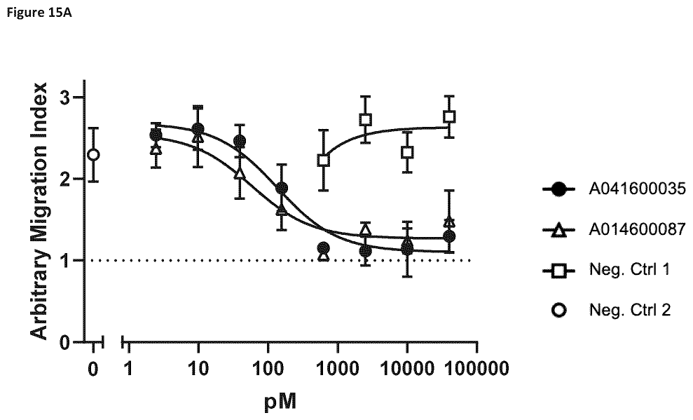

A041600035 can block the activity of both ligands on human CX3CR1 to an equal extent with an IC50 of 6 nM (see Table B-12 and FIG. 14). These experiments also show that mouse CX3CL1 is about 10 to 40-fold less potent to induce ERK phosphorylation compared to human CX3CL1 (both on human or mouse CX3CR1). The surrogate antibody recognizing mouse CX3CR1 (AB5715, Biolegend Cat. No. 149002) blocked CX3CL1 induced ERK phosphorylation in CHO K1 mouse CX3CR1 cells with an IC50 of 0.8 to 1.6 nM (see Table B-12 and FIG. 14).

TABLE B-12
Inhibition of ERK phosphorylation induced by human (hu) or mouse (m) fractalkine (CX3CL1).

| | | A041600035 | | Anti-mouse CX3CR1 (N = 3) | |
|---|---|---|---|---|---|
| (N = 5) | | (N = 4) | | (Biolegend, Cat No. 149002) | |
| huCX3CL1 | mCX3CL1 | huCX3CL1 | mCX3CL1 | huCX3CL1 | mCX3CL1 |
| EC50 (M) | EC50 (M) | IC50 (M) | IC50 (M) | IC50 (M) | IC50 (M) |

| | | | | | |
|---|---|---|---|---|---|
| huCX3CR1 | 2.0E−09 | 7.3E−08 | 5.9E−09 | 5.6E−09 | No activity |

| | | | | | |
|---|---|---|---|---|---|
| mCX3CR1 | 2.5E−09 | 2.3E−08 | No activity | 7.5E−10 | 1.6E−10 |

Italics and underlined: indicative value

### 8.3 Affinity Determination

The in-solution affinity of A041600035 for cell expressed human and cyno CX3CR1 was determined via the solution equilibrium kinetic exclusion KD measurement method MSD-SET (Meso Scale Discovery-solution equilibration titration) in the presence of 30 μM HSA. The affinity was determined on 3 independent assay occasions.

The geometric means of the 3 independent experiments are shown in Table B-13. There is no difference in affinity for human and cyno CX3CR1.

TABLE B-13
In-solution affinity (KD) of A041600035
for human and cyno CX3CR1.

| Human CX3CR1 (N = 3) | Cyno CX3CR1 (N = 3) |
|---|---|
| 1.3E−10M | 1.1E−10M |

### 8.4 Characterization of A041600035 in Blocking Fractalkine-Mediated Monocyte Chemotaxis from Healthy and Lupus Nephritis Patients

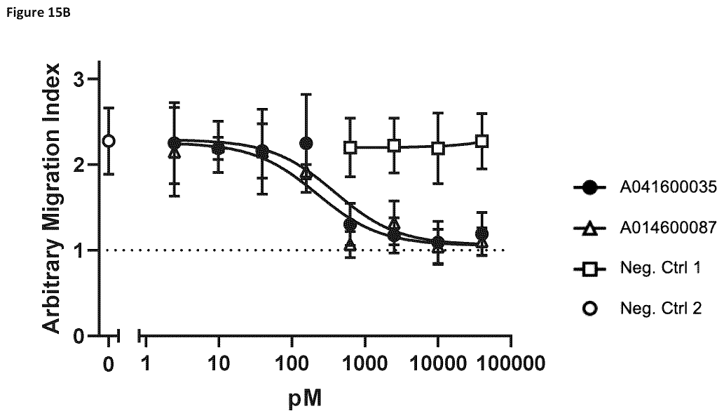

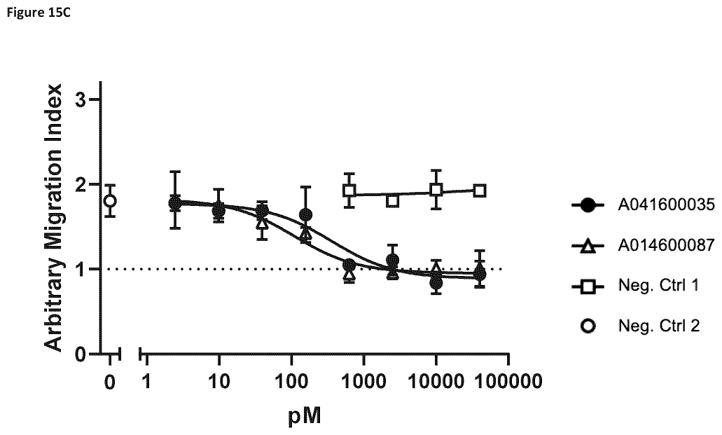

Pan monocytes from healthy donors (HemaCare) were thawed at 37° C., washed with RPMI containing 10% FBS, resuspended in RPM11640 containing 10% FBS and 25 ug/mL DNAse I (Sigma-Aldrich), and rested for 1 h at 37 C, 5% CO₂. Monocytes were spun down and stained with anti-human CD14-APC (Biolegend) and anti-human CD16-PE (Biolegend) in RPM11640 containing 10% FBS at 1:100 dilution for 10 min at room temperature. Monocytes were then washed and resuspended in RPM11640 containing 1% FBS at the concentration of 5×10E6/mL. Monocyte suspension was incubated for 10 min at room temperature at 1:1 volume ratio with either A041600035, A041600087, or negative control (IRR00163; VHH reference) or media at different concentrations. Full-length recombinant fractalkine (R&D systems) was diluted at 50 ng/mL in RPM11640 containing 1% FBS and 500 uL of diluted fractalkine was added in the lower chambers of the transwell (24 well, Corning). Then 100 uL of monocyte suspension was gently added to the top chambers of the transwell. The transwell plate was placed in the incubator and cultured for 3 h at 37 C, 5% C02. Top chambers were then removed, and lower chambers were mixed gently by pipetting. Flowcytometric analysis was performed to count monocyte subsets (Classical: CD14+CD16−, Intermediate: CD14−CD16+, and Non-classical: CD14−CD16+) in 100 uL of suspension from the lower chambers. Arbitrary chemotaxis index was shown as a fold change to the media control wells: (cell number)/(cell number in media control) (Table B-14; FIG. 15).

TABLE B-14
Descriptive table of arbitrary migration index for
A041600035 with different monocyte subsets from
healthy volunteers (n = 3 for all conditions)

| | Concen- | Classical | Intermediate | Non-classical |
|---|---|---|---|---|
| | tration | Monocytes | Monocytes | Monocytes |
| Compound | (pM) | GM | GM | GM |
| IRR00163 | 625 | 2.17 | 2.15 | 1.91 |
| | 2500 | 2.70 | 2.18 | 1.80 |
| | 10000 | 2.30 | 2.11 | 1.91 |
| | 40000 | 2.74 | 2.23 | 1.92 |
| A041600035 | 625 | 1.15* | 1.26* | 1.05* |
| | 2500 | 1.09** | 1.14* | 1.08* |
| | 10000 | 1.05* | 1.04** | 0.82** |
| | 40000 | 1.27* | 1.13** | 0.91* |

Adjusted p-values were obtained with Dunnett's test at fixed concentration after a two-way ANOVA with factors compound and concentration and their interactions on the log-transformed arbitrary migration index parameter for the relevant index
*IRR00163 vs SAR445611 adjusted P value < 0.05;
**IRR00163 vs SAR445611 adjusted P value < 0.001
GM = Geometric mean

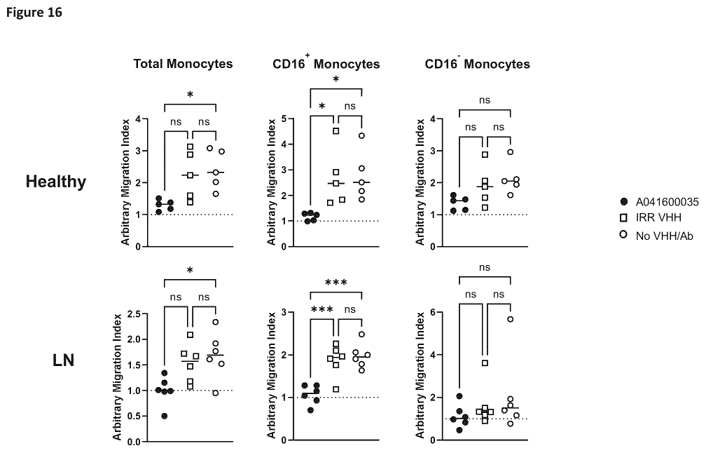

Blood samples from lupus nephritis patients (LN disease class 2-5) were provided by Dr. Ian R. Rifkin from Boston Medical Center, MA. PBMC were isolated from fresh blood using SepMate (Stemcell), and pan-monocytes were enriched using pan-monocyte enrichment kit (Miltenyi Biotech). Isolated monocytes were used for transwell assay in triplicates and arbitrary chemotaxis index was calculated as described above (Table B-15 and FIG. 16).

TABLE B-15
Arbitrary migration index for A041600035 with the different monocyte subsets
from healthy volunteers and LN patients (n = 3 for all conditions).

| | | Fold inhibition | | |
|---|---|---|---|---|
| | | (A041600035 VS | | Adjusted |
| Population | Monocyte Cell type | IRR VHH) | 95% CI on FI | p-value |
| Healthy | CD16+ Monocytes | 2.34 | [1.71; 3.21] | <.0001 |
| | CD16− Monocytes | 1.50 | [1.10; 2.06] | 0.0133 |
| | Total Monocytes | 1.82 | [1.33; 2.49] | 0.0014 |
| LN | CD16+ Monocytes | 1.90 | [1.46; 2.46] | 0.0001 |
| | CD16− Monocytes | 1.71 | [1.32; 2.22] | 0.0006 |
| | Total Monocytes | 1.71 | [1.32; 2.22] | 0.0006 |

P-values are obtained with mixed-effect model on log-transformed arbitrary migration index values applying Bonferroni-Holm adjustment per population (correction for multiple monocyte cell type testing). Abbreviations: CD = cluster of differentiation, CI = Confidence Interval, LN = lupus nephritis

### Example 9: Evaluation of Selected Multivalent ISVD Constructs in a Non-Accelerated Model of NTS-Induced Glomerulonephritis in Knock-In hCX3CR1 Transgenic Mice

### 9.1 Nephrotoxic Nephritis (NTN) Model

The non-accelerated NTN model in mice is described in the literature as a model of acute glomerulonephritis where nephritis is initiated by the administration of an anti-glomerular serum (ie, nephrotoxic antiserum [NTS]). The NTS binds and deposits in kidneys' glomeruli and impairs the glomerular filtration barrier resulting in proteinuria and inflammation (Ougaard et al. 2018, Int. J. Nephrol. 2018: 8424502: 1-12). In view of the lack of mouse cross reactivity of the multivalent ISVD constructs, NTN studies were performed on hCX3CR1 KI transgenic mice. A hCX3CR1 KI (C57BL/6) strain was developed by Boehringer Ingelheim. Female hCX3CR1 KI (C57BL/6) mice received a single injection of sheep anti-rat glomeruli serum with a 7-day observation period (Example 9.4) or a 21-day (Example 9.5) observation period post-NTS administration.

### 9.2 Immunohistochemical Procedures

Tissue renal damage mediated by glomerulus-infiltrating cells is a key pathogenic early event expected to be detectable in this model (Ougaard et al. 2018, Int. J. Nephrol. ID 8424502: 1-12). Efficacy of treatment with the multivalent ISVD constructs on compartment-specific monocyte/macrophage and T cell types infiltration in renal cortex was analyzed using different biomarkers used in literature (Hochheiser et al. 2013, J. Clin. Invest. 123: 4242-4254; Guo et al. 2019, Faseb J. 33: 2359-2371; Sung et al. 2017, J. Immunol. 198: 2589-2601). Treatment with Dexamethasone (daily administrations at a unique dose) and an irrelevant VHH (IRR00163) not binding to any mouse protein were used as controls to evaluate the effect of treatment.

CD11b and Mac-2 antibodies were used to differently detect both infiltrating blood-derived monocytes and macrophages that could be distinct to F4/80 positive resident and more mature macrophages found in the interstitial and periglomerular regions (Geissmann et al. 2003, Immunity 19: 71-82; Steinmetz et al. 2009, J. Immunol. 183: 4693-704; Bideak et al. 2018, Kidney Int. 93: 826-41). Interestingly, galectin 3 protein (recognized by Mac-2 antibody) expression in renal tissue and serum galectin-3 levels were reported to be elevated in patients with LN versus healthy controls and identified as possible disease activity biomarkers in LN (Kang et al. 2009, Lupus 18: 22-28). Additional CD4 and CD8 immunostaining was used to detect a subpopulation of lymphocyte T cells that are also considered important for LN pathogenesis (Couzi et al. 2007, Arthritis Rheum. 56: 2362-2370).

### 9.2.1 FFPE Kidney Tissue Preparation

At day 7 and/or 21 of animal's necropsy, left kidney dedicated for immunohistochemical studies were divided in its middle part into two pieces along the longitudinal axis (in a horizontal section in the middle of the kidney). The two halves were placed for 2 days in 4% w/v formaldehyde buffered at pH 6.9 RS solution (Sigma Aldrich) at room temperature. The fixation step was stopped for kidney pieces kept in appropriate cassettes by rinsing in PBS buffer (1×PBS Gibco pH7.4, Cat #10010-031) for 2×5 min before dehydration and embedding in paraffin.

Paraffin-embedded tissue blocks containing kidney of hCX3CR1 KI mice were sectioned at 3 μm thickness on a microtome and the sections were transferred onto glass slides suitable for IHC. Immunostainings were performed on dewaxed slides using either Ventana Discovery XT or Ventana ULTRA automated system according to manufacturer's instructions (Ventana Medical Systems, Inc, USA).

### 9.2.2 Immunostaining

Different primary antibodies were used to detect myeloid cell types on murine FFPE kidney tissues (i.e., anti-F4/80, anti-CD11b and anti-Mac-2/Galectin-3 antibodies) in different IHC assays performed on either Ventana Discovery XT or Ventana ULTRA automated systems. Anti-CD4 and/or anti-CD8 antibodies were used to detect lymphocyte T cells. Detections systems with different degrees of amplification (manufactured by Ventana Medical System Inc) were used according to the primary antibody used.

The F4/80 (D2S9R) XP© antibody, a rabbit monoclonal Immunoglobulin G (IgG) (Cell Signaling Technology, reference #70076) is usually used to detect macrophage cell types. Detection system was Biotin free Discovery anti-rabbit UltraMap™ horseradish peroxidase (HRP) conjugate (760-4315, Ventana Medical Systems, Inc, USA).

The Mac-2/Galectin-3 antibody, a rat monoclonal IgG2a antibody (Clone M3/38, Cedarlane, reference CL8942AP) is usually used to detect monocyte/macrophage cell types. Detection systems were a secondary antibody (linker) corresponding to Rabbit@rat IgG (Clone R18-2, Abcam, reference ab125900) and a biotin free Discovery anti-Rabbit OmniMap horseradish peroxidase (HRP) conjugate (760-4311, Ventana Medical Systems, Inc, USA).

The CD11b [EPR1344] rabbit antibody, a rabbit monoclonal IgG antibody (Abcam, reference ab133357) is usually used to detect monocyte cell types. Detection system were Discovery anti-Rabbit HQ (760-4815, Ventana Medical Systems, Inc, USA) and anti-HQ HRP multimer (760-4820, Ventana Medical Systems, Inc, USA).

The CD4 rat monoclonal IgG1 antibody (Clone 4SM95, Invitrogen, reference 14-9766-82) is used to detect a cell surface receptor on a subpopulation of lymphocyte T cells. Detection systems were a secondary antibody (linker) corresponding to Rabbit@rat IgG (Clone R18-2, Abcam, reference ab125900), Discovery anti-Rabbit HQ (760-4815, Ventana Medical Systems, Inc, USA) and Discovery anti-HQ HRP multimer (760-4820), Ventana Medical Systems, Inc, USA).

The CD8a rat monoclonal IgG2a antibody (Clone 4SM15, Invitrogen, reference 14-0808-82) is used to detect a cell surface receptor on a subpopulation of lymphocyte T cells. Detection systems were a secondary antibody (linker) corresponding to Rabbit@rat IgG (Clone R18-2, Abcam, reference ab125900), Discovery anti-Rabbit HQ (760-4815, Ventana Medical Systems, Inc, USA) and Discovery anti-HQ HRP multimer (760-4820), Ventana Medical Systems, Inc, USA).

Immunostainings were finalized using the universal 3,3′diaminobenzidine (DAB) chromogenic detection kit (760-159, Ventana Medical Systems, Inc, USA) for CD11b, F4/80 and Mac-2/Galectin-3 markers. For CD4 and CD8 markers, Discovery purple kit (760-229) was used. A counterstaining step was also done with the hematoxylin II (790-2208, Ventana Medical Systems, Inc, USA) and bluing reagent was applied (760-2037, Ventana Medical Systems, Inc, USA). Stained slides were dehydrated, and cover slipped with cytoseal XYL (8312-4, Richard-Allan scientific, USA).

### 9.3 Data Analysis

### 9.3.1 Image Analysis

Quantitative image analysis was performed on an image analysis platform (HALO, Indica Labs) using Multiplex IHC module based on automated cell count. Analysis was done either within the whole cortex (in this case, values of mean density of positive cells per treatment group were analyzed) or in glomeruli only (in this case, values of mean number of positive cells in glomeruli analyzed) considered as 2 different regions of interest. For CD11b/CD4 double staining, glomeruli were automatically segmented using HALO AI Dense Net neural network (with a range of 130 to 214 glomeruli detected per animal at 7 days and 143 to 284 glomeruli detected per animal for analysis at day 21). For single Mac-2/Galectin-3 (day 7) or double CD8 and Mac-2/Galectin-3 (day 21) staining, 80 glomeruli (7-day study) or 100 glomeruli (21-day study), manually delineated over the whole cortex, were analyzed for each animal.

### 9.3.2 Statistical Analysis

One-way ANOVAs have been fitted per IHC parameter on log-transformed responses to relieve variance heterogeneity. By the log-transformation, effect interpretations are in terms of ratios (fold changes) of GMs. Dunnett's corrections (both on p-value and on Cl bounds) have been applied per IHC parameter for the 2 day and 4 treatment effect comparisons to control the family-wise error rate. Analysis results are represented per IHC parameter including fold decreases and significance scorings.

Analyses has been performed in SAS Enterprise Guide v8.2.0 with SAS© software version 9.4 for Windows 10 Enterprise 64-bit. Fold decreases and significance scoring were scripted in RStudio v1.1.453 with R v4.1.3 using packages tidyverse, rstatix, ggplot2, ggpubr, plotly and EnvStats.

### 9.4 Evaluation of Renal Immunohistochemical Immune Cell Markers in a 7-Day Non-Accelerated Model of NTS-Induced Glomerulonephritis in Knock-In hCX3CR1 Transgenic Mice in Response to A041600035 Administration

### 9.4.1 Study Design

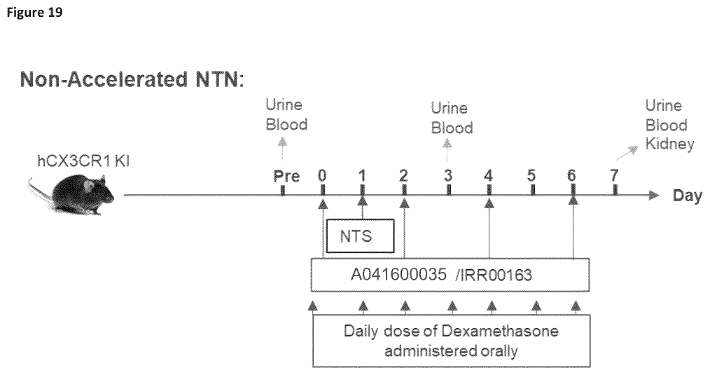

NTS (sheep anti-rat glomeruli serum; PTX-001S-Ms, Probetex) was injected via intraperitoneal route on Day 1 to induce non-accelerated NTN. The dose levels and route of administration of NTS were based on literature on this model (Ougaard et al. 2018, Int. J. Nephrol. ID 8424502: 1-12; Hochheiser et al. 2013, J. Clin. Invest. 123: 4242-4254; Guo et al. 2019, Faseb J. 33: 2359-2371) and were further effectively determined in an optimization study. The study contained five treatment groups and each treatment group initially included 10 female hCX3CR1 KI mice injected at day 1 with 240 μl NTS. The protocol of the experimental in vivo study design is outlined in FIG. 19.

The treatments were administered as detailed in Table B-16. A041600035 was administered on days 0, 2, 4 and 6 intraperitoneally (IP). Dexamethasone (0.3 mg/kg) was administered daily per oral (PO) gavage.

TABLE B-16
Treatment groups and dosing details
of 7-day non-accelerated NTN study.

| | | | # of |
|---|---|---|---|
| Group | Treatment | Planned Dose Level | mice |
| 1 | NTS/IRR00163 | 30 mg/kg; day 0, 2, 4 & 6 IP | 10 |
| 2 | NTS/A041600035 | Dose 1; day 0, 2, 4 & 6 IP | 10 |
| 3 | NTS/A041600035 | Dose 2; day 0, 2, 4 & 6 IP | 10 |
| 4 | NTS/A041600035 | Dose 3; day 0, 2, 4 & 6 IP | 10 |
| 5 | NTS/Dexamethasone | 0.3 mg/kg, daily; PO | 10* |

*1 animal in the Dexamethasone group has been euthanized before day 7.

### 9.4.2 Microscopic Observations

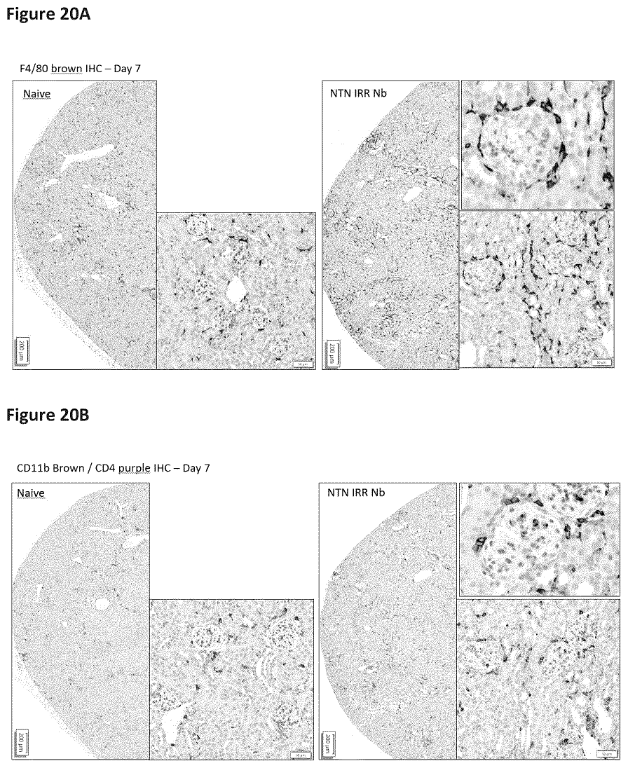

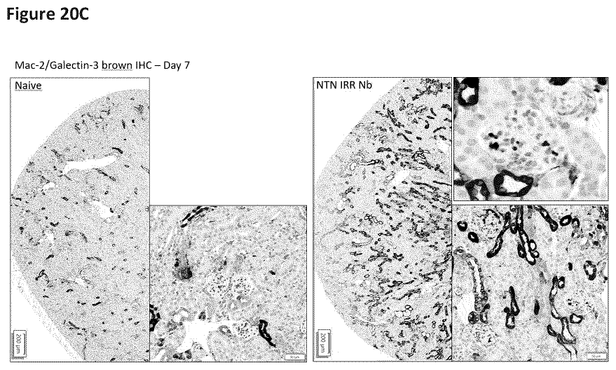

Marked alterations of the distribution pattern and density of F4/80, CD11b and Mac-2/Galectin-3 positive cells over renal tissue were microscopically observed for all hCX3CR1 KI mice of NTS/IRR00163 group when compared with the 2 naive non treated hCX3CR1 KI mice (FIG. 20). As expected, NTS injection induced at day 7 a prominent overall accumulation of F4/80 positive cells over the entire cortical area and almost exclusively in peri-glomerular and tubulointerstitial regions. In contrast, increase in CD11b and Mac-2/Galectin-3 positive cells at day 7 was mainly detectable in glomeruli and in more localized subareas of the tubulo-interstitium as compared to F4/80. Of note, Mac-2/Galectin-3 protein expression was also markedly increased in tubular epithelium within the renal-interstitium, as already reported in cases of renal pathology (Henderson et al. 2008, Am. J. Pathol. 172: 288-298).

### 9.4.3 Quantitative Analysis

Five IHC marker/location variables, and—hereafter called IHC parameters—have been analyzed: 1) ‘Mean density of F4/80+ cells in entire cortex (per mm²)’; 2) ‘Mean density of CD11b+ cells in entire cortex (per mm²)’; 3) ‘Mean density of CD4+ cells in entire cortex (per mm²)’; 4) ‘Mean number of CD11b+ cell per glomerulus in cortex’; 5) ‘Mean number of Mac-2/Galectin-3+ cell per glomerulus in cortex’.

The IHC analysis of tissue from study revealed that 7 days after NTS administration, there was a dose dependent significant reduction in the number of monocytes/macrophages (CD11b+; Mac-2/Galectin-3+) per glomerulus in response to A041600035 treatment compared to the mice receiving negative control with fold decrease (FD) in excess of 2.0 (Table B-17). In comparison, daily dexamethasone administration for 7 consecutive days at 0.3 mg/kg daily IP administration induced a more limited protective effect on mean number of positive cells per cortical glomerulus for both CD11b (estimated average of 1.35 FD, p=0.0105) and Mac-2/Galectin-3 (estimated average of 1.50 FD, p=0.0058) markers.

No change in the mean density of CD4+ cells in the cortex could be detected with any of the treatments.

TABLE B-17
Fold decrease treatment effects on mean number of CD11b+ and Mac-2/Galectin
3+ cells per glomerulus in cortex at Day 7 compared to NTS IRR00163 group.

| | | CD11b+ | Adjusted | Mac-2/Galectin3+ | Adjusted |
|---|---|---|---|---|---|
| Treatment Group | N | FD^{a} | p-value | FD^{a} | p-value |
| NTS/dexamethasone | 9 | 1.35 | 0.0105 | 1.50 | 0.0058 |
| NTS/A041600035 | 10 | 1.35 | 0.0098 | 1.44 | 0.0116 |
| Dose 1 | | | | | |
| NTS/A041600035 | 10 | 1.86 | <0.0001 | 1.73 | 0.0001 |
| Dose 2 | | | | | |
| NTS/A041600035 | 10 | 2.24 | <0.0001 | 2.46 | <0.0001 |
| Dose 3 | | | | | |

^{a}FD (fold decrease) compared to NTS/IRR00163

Based on IHC evaluation of CD11b, Mac-2/Galectin-3 and F4/80 immune cell markers, this study suggest that A041600035 could induce a dose-dependent decrease in the number of monocyte/macrophage cell types in kidney tissue from a non-accelerated NTN model in hCX3CR1 KI mice, at day 7 post-NTS injection. The fold decreased observed for the CD11b and Mac-2/Galectin-3 IHC markers for the 2 highest concentrations of A041600035 were more pronounced as compared to the Dexamethasone tested dose.

### 9.5 Evaluation of Renal Immunohistochemical Immune Cell Markers in a 21-Day Non-Accelerated Model of NTS-Induced Glomerulonephritis in Knock-In hCX3CR1 Transgenic Mice in Response to A041600035 Administration

### 9.4.1 Study Design

NTS (sheep anti-rat glomeruli serum; PTX-001S-Ms, Probetex) was injected via intraperitoneal route on Day 1 to induce non-accelerated NTN. The dose levels and route of administration of NTS were based on literature on this model (Ougaard et al. 2018, Int. J. Nephrol. ID 8424502: 1-12; Hochheiser et al. 2013, J. Clin. Invest. 123: 4242-4254; Guo et al. 2019, Faseb J. 33: 2359-2371) and were further effectively determined in an optimization study. The study contained eleven treatment groups and each treatment group included 7 female hCX3CR1 KI mice injected at day 1 with 240 I NTS, except for naive group (without any NTS injection) which contained 2 animals. For all treatment groups, dosing was initiated at Day 0 and necropsy was done at day 7 for one part of animals (corresponding to group 1 to 6) and then at day 21 (corresponding to group 7 to 11) post unique injection of NTS. The protocol of the experimental in vivo study design is outlined in FIG. 21. The treatments were administered as detailed in Table B-18.

TABLE B-18
Treatment groups and dosing details of 7-day
and 21-day non-accelerated NTN mouse model.

| | | | |
|---|---|---|---|
| Group | Treatment | Dose Level | # of mice |

Day 7 Necropsy

| | | | |
|---|---|---|---|
| 1 | Naive | — | 2 |
| 2 | NTS/Dexamethasone | 0.5 mg/kg, daily; IP | 7 |
| 3 | NTS/Cyclophosphamide | 30 mg/kg, weekly; IP | 7 |
| 4 | NTS/Mycophenolate mofetil | 100 mg/kg, daily, PO | 7 |
| 5 | NTS/IRR00163 | 30 mg/kg, every two days; IP | 7* |
| 6 | NTS/A041600035 | Dose 3, every two days; IP | 7 |

Day 21 Necropsy

| | | | |
|---|---|---|---|
| 7 | NTS/Dexamethasone | 0.5 mg/kg, daily; IP | 7 |
| 8 | NTS/Cyclophosphamide | 30 mg/kg, weekly; IP | 7 |
| 9 | NTS/Mycophenolate mofetil | 100 mg/kg, daily, PO | 7 |
| 10 | NTS/IRR00163 | 30 mg/kg, every two days; IP | 7 |
| 11 | NTS/A041600035 | Dose 3, every two days; IP | 7 |

*one animal is missing for IHC studies because of an issue during the tissue processing.

### 9.4.2 Microscopic Observations

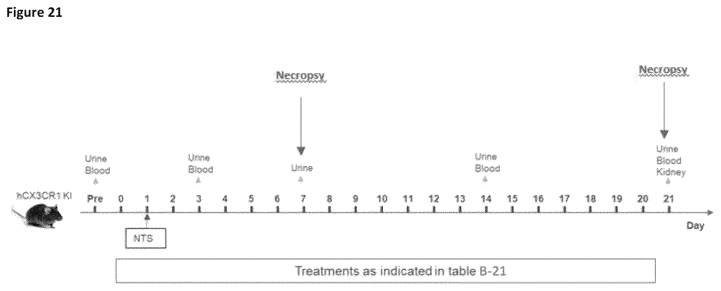

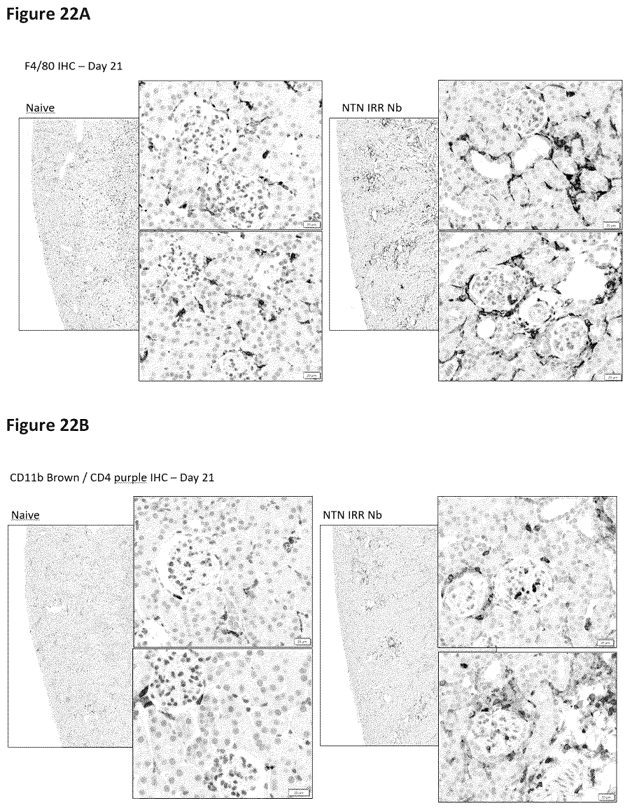

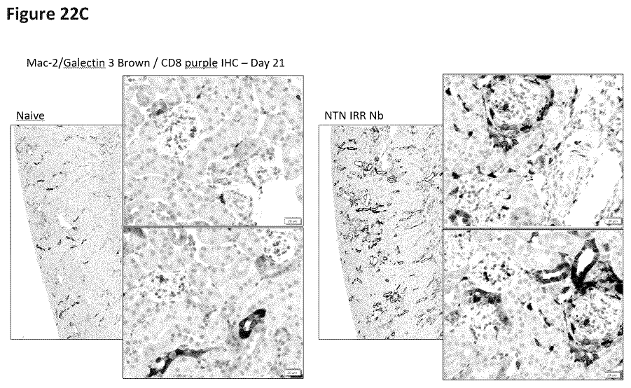

As already described in Example 9.3, at the early timepoint of 7 days post-unique injection of NTS, marked alterations of the distribution pattern and density of F4/80, CD11b, Mac-2/Galectin-3 and CD4 positive cells over renal tissue were microscopically observed for all hCX3CR1 KI mice treated with NTS and IRR00163 Nb, when compared with the 2 naive non NTS-treated hCX3CR1 KI mice. At 21 days after injection, NTS qualitatively induced similar microscopically alterations, with a noticeable increase of the density of CD4 positive cells into the whole renal cortex of hCX3CR1 KI mice treated with NTS and IRR00163 (FIG. 22). A prominent overall accumulation of F4/80 positive cells over the entire cortical area and almost exclusively in peri-glomerular and tubulointerstitial regions was clearly still detectable at day 21. Densities of CD11b and Mac-2/Galectin-3 positive cells were increased in glomeruli and in more localized (as compared to F4/80) subareas of the tubulointerstitium. Of note, Mac-2/Galectin-3 protein expression was still increased at day 21 in tubular epithelium within the renal interstitium, as already reported in cases of renal pathology (Henderson et al. 2008, Am. J. Pathol. 172: 288-298). In contrast to other markers at day 21, overall alteration of CD8 marker induced at day 21 post NTS injection was limited.

### 9.4.3 Quantitative Analysis

Various IHC markers/location variables—hereafter called IHC parameters—have been selected for analysis at day 7 and 21 days after first IP injections or oral gavage considering 5 treatment groups. The following 6 have been selected for analysis at day 7: ‘Mean density of F4/80+ cells in entire cortex (mm²)’, ‘Mean density of CD4+ cells in entire cortex (mm²)’, ‘Mean density of CD11b+ cells in entire cortex (mm²)’, ‘Mean number of CD4+ cells per glomerulus in cortex’, ‘Mean number of CD11b+ cells per glomerulus in cortex’ and ‘Mean number of Mac-2/Galectin-3+ cells per glomerulus in cortex’. Analysis at day 21 additionally includes ‘Mean density of CD8+ cells in entire cortex (mm²)’ and ‘Mean number of CD8+ cells per glomerulus in cortex’ IHC markers. Each of these parameters have been evaluated by contrasting the “Dexamethasone (0.5 mg/kg)”, “Cyclophosphamide (30 mg/kg)”, “Mycophenolate mofetil (100 mg/kg)” and ‘NTS/A041600035′ treatment groups with the ‘NTS/IRR00163′ (30 mg/kg)’ control group.

The IHC analysis of tissue, including Day 7 and Day 21 after NTS administration, revealed a distinct pharmacological effect for A041600035 with a marked and significant reduction in CD11b+ and Mac-2/Galectin-3 monocytes/macrophages per glomerulus. In comparison, dexamethasone and to a lesser degree mycophenolate mofetil decreased CD4+ lymphocyte T cell populations. (Table B-19).

TABLE B-19
Summary of fold-decrease treatment effects on mean density of F4/80+, CD11b+, Mac-
2/Galectin-3, CD4+ and CD8+ cells per glomerulus at Days 7 and 21.

| | F4/80 | | CD11b | | Mac-2/Galectin-3 | | CD4 | | CD8 | |
|---|---|---|---|---|---|---|---|---|---|---|
| Glomeruli in cortex^{a} | 7 d | 21 d | 7 d | 21 d | 7 d | 21 d | 7 d | 21 d | 7 d | 21 d |
| NTS/Dexamethasone | NA¹ | NA¹ | 1.22 | 1.11 | 1.48 | 1.18 | 1.27 | 2.13* | NA² | 1.23 |
| NTS/Cyclophosphamide | NA¹ | NA¹ | 1.05 | 1.16 | 0.95 | 0.97 | 0.67 | 1.29 | NA² | 1.30 |
| NTS/Mycophenolate | NA¹ | NA¹ | 1.05 | 0.71 | 1.29 | 0.81 | 1.00 | 1.19 | NA² | 1.09 |
| NTS/A041600035 | NA¹ | NA¹ | 2.02^{4*} | 2.00^{3*} | 2.50^{2*} | 2.43^{4*} | 1.03 | 1.15 | NA² | 1.05 |

NA¹: Not Assessed in view of the intense staining on endothelial cells
NA²: Not Assessed due to operational issues
Significance codes for
*adjusted p values:
*p < 0.05;
²*p < 0.01;
³*p < 0.001 and
⁴*p < 0.0001
^{a}FD (fold decrease) compared to NTS/IRR00163

Overall, these results show a profound effect of A041600035 on CD11b+ and Mac-2/Galectin-3+ infiltrating blood-derived monocytes and macrophages in the kidney cortex and more significantly in the glomerular area of NTN animals at 7 and 21 days post-NTS injection. A less pronounced but significant effect was only observed on F4/80+ macrophages in the 7-day study, possibly reflecting an activity on the blood-derived subset of this mixed population of infiltrating and tissue resident macrophages. No effect of A041600035 could be detected on CD4 and CD8 T cell populations.

The effect of A041600035 was well differentiated from dexamethasone especially at 21-days after NTS injection. In comparison, Dexamethasone treatment induced significant decreases in both CD4 and CD8 positive T cell populations at 21 days after NTS injection but had no effect on monocytes and macrophages markers. In the same model, Mycophenolate mofetil treatment only had a significant effect on the CD4 positive T cell population at 21 days post NTS injection and when the whole kidney cortex was measured.

No significant effect could be detected on any of the selected IHC markers following cyclophosphamide treatment in this model.

### Example 10: Determination of the Pharmacokinetics (PK) Profile

A041600035 or A041600087 were administered as a single dose to Cynomolgus monkeys by intravenous infusion. Blood samples were collected at different timepoints after dosing. The concentrations of A041600035 or A041600087 were measured by ligand binding assay. The concentration—time profiles were plotted (FIG. 18).

The different pK profile observed for A041600087 at higher dose compared to lower dose might be an indication of target mediated drug deposition (TMDD). For A041600035 this TMDD is not observed. Moreover, at similar dose, the exposure for A041600035 was higher compared to the exposure for A041600087.

### 7. INDUSTRIAL APPLICATION

The present technology is capable of exploitation in industry. Applications and practical exploitation in industry may be derived from the present description by the skilled person's general knowledge.

### 8. SEQUENCE LISTING

The term “ID” in the tables below (last column) refers to “SEQ ID NO” as used herein.

TABLE A-1
Monovalent anti-CX3CR1 ISVD sequences having the structure
FR1 - CDR1 - FR2 - CDR2 - FR3 - CDR3 - FR4 in which FR1 to
FR4 refer to framework regions and CDR1 to CDR3 refer to
complementarity determining regions.

| Name | | |
|---|---|---|
| [further | | |
| info] | Sequence | ID |
| 54A12 | EVQLVESGGGSVQAGESLRLSCAASGSIFSSNAMAWYRQAPGKQRDL | 1 |
| | VAVINSVGITKYADSVKGRFTISGDNAKNTVYLQMNSLKPEDTAVYY | |
| | CTSDARRGWDTRYWGQGTQVTVSS | |
| 54A12_SO | EVQLVESGGGSVQPGGSLRLSCAASGSIFSSTAMAWYRQAPGKQRDL | 2 |
| | VAVISTVGITKYADSVKGRFTISRDNAKNTVYLQMNSLRPEDTALYY | |
| | CTSDARRGWDTRYWGQGTLVTVSS | |
| 54A12_SO | DVQLVESGGGSVQPGGSLRLSCAASGSIFSSTAMAWYRQAPGKQRDL | 3 |
| (E1D) | VAVISTVGITKYADSVKGRFTISRDNAKNTVYLQMNSLRPEDTALYY | |
| | CTSDARRGWDTRYWGQGTLVTVSS | |
| 66B02 | EVQLVESGGGSVQAGESLRLSCAASGSIFSSNAMAWYRQAPGKRRDL | 4 |
| | VAAINSVGVTKYADSVKGRFTISRDNAKNTVYLQMNSLKPEDTAVYY | |
| | CTSDPRRGWDTRYWGQGTQVTVSS | |
| 66B02_SO | EVQLVESGGGSVQPGGSLRLSCAASGSIFSSTAMAWYRQAPGKRRDL | 5 |
| | VAAINTVGVTKYADSVKGRFTISRDNAKNTVYLQMNSLRPEDTALYY | |
| | CTSDPRRGWDTRYWGQGTLVTVSS | |
| 66B02_SO | DVQLVESGGGSVQPGGSLRLSCAASGSIFSSTAMAWYRQAPGKRRDL | 6 |
| (E1D) | VAAINTVGVTKYADSVKGRFTISRDNAKNTVYLQMNSLRPEDTALYY | |
| | CTSDPRRGWDTRYWGQGTLVTVSS | |
| 307 | DVQLVESGGGLVQPGGSLRLSCAASGSIFSSTAMAWYRQAPGKRRDL | 7 |
| | VAAISTVGVTKYADSVKGRFTISRDNSKNTVYLQMNSLRPEDTAVYY | |
| | CTSDPRRGWDTRYWGQGTLVTVSS | |
| 307 (D1E) | EVQLVESGGGLVQPGGSLRLSCAASGSIFSSTAMAWYRQAPGKRRDL | 8 |
| | VAAISTVGVTKYADSVKGRFTISRDNSKNTVYLQMNSLRPEDTAVYY | |
| | CTSDPRRGWDTRYWGQGTLVTVSS | |

TABLE A-2
Anti-CX3CR1 ISVD sequence parts: CDRs according to
AbM numbering and frameworks.

| Name [further info] | Sequence | ID |
|---|---|---|
| 54A12_FR1 [CX3CR1, FR1, | EVQLVESGGGSVQAGESLRLSCAAS | 16 |
| AbM] | | |
| 54A12_CDR1 [CX3CR1, | GSIFSSNAMA | 17 |
| CDR1, AbM] | | |
| 54A12_FR2 [CX3CR1, FR2, | WYRQAPGKQRDLVA | 11 |
| AbM] | | |
| 54A12_CDR2 [CX3CR1, | VINSVGITK | 20 |
| CDR2, AbM] | | |
| 54A12_FR3 [CX3CR1, FR3, | YADSVKGRFTISGDNAKNTVYLQMNSLKPEDTAVYYCTS | 25 |
| AbM] | | |
| 54A12_CDR3 [CX3CR1, | DARRGWDTRY | 14 |
| CDR3, AbM] | | |
| 54A12_FR4 [CX3CR1, FR4, | WGQGTQVTVSS | 28 |
| AbM] | | |
| 54A12_SO_FR1 [CX3CR1, | EVQLVESGGGSVQPGGSLRLSCAAS | 9 |
| FR1, AbM] | | |
| 54A12_SO_CDR1 [CX3CR1, | GSIFSSTAMA | 10 |
| CDR1, AbM] | | |
| 54A12_SO_FR2 [CX3CR1, | WYRQAPGKQRDLVA | 11 |
| FR2, AbM] | | |
| 54A12_SO_CDR2 [CX3CR1, | VISTVGITK | 12 |
| CDR2, AbM] | | |
| 54A12_SO_FR3 [CX3CR1, | YADSVKGRFTISRDNAKNTVYLQMNSLRPEDTALYYCTS | 13 |
| FR3, AbM] | | |
| 54A12_SO_CDR3 [CX3CR1, | DARRGWDTRY | 14 |
| CDR3, AbM] | | |
| 54A12_SO_FR4 [CX3CR1, | WGQGTLVTVSS | 15 |
| FR4, AbM] | | |
| 54A12_SO_(E1D) FR1 | DVQLVESGGGSVQPGGSLRLSCAAS | 29 |
| [CX3CR1, FR1, AbM] | | |
| 66B02_FR1 [CX3CR1, FR1, | EVQLVESGGGSVQAGESLRLSCAAS | 16 |
| AbM] | | |
| 66B02_CDR1 [CX3CR1, | GSIFSSNAMA | 17 |
| CDR1, AbM] | | |
| 66B02_FR2 [CX3CR1, FR2, | WYRQAPGKRRDLVA | 18 |
| AbM] | | |
| 66B02_CDR2 [CX3CR1, | AINSVGVTK | 139 |
| CDR2, AbM] | | |
| 66B02_FR3 [CX3CR1, FR3, | YADSVKGRFTISRDNAKNTVYLQMNSLKPEDTAVYYCTS | 140 |
| AbM] | | |
| 66B02_CDR3 [CX3CR1, | DPRRGWDTRY | 21 |
| CDR3, AbM] | | |
| 66B02_FR4 [CX3CR1, FR4, | WGQGTQVTVSS | 28 |
| AbM] | | |
| 66B02_SO_FR1 [CX3CR1, | EVQLVESGGGSVQPGGSLRLSCAAS | 9 |
| FR1, AbM] | | |
| 66B02_SO_CDR1 [CX3CR1, | GSIFSSTAMA | 10 |
| CDR1, AbM] | | |
| 66B02_SO_FR2 [CX3CR1, | WYRQAPGKRRDLVA | 18 |
| FR2, AbM] | | |
| 66B02_SO_CDR2 [CX3CR1, | AINTVGVTK | 19 |
| CDR2, AbM] | | |
| 66B02_SO_FR3 [CX3CR1, | YADSVKGRFTISRDNAKNTVYLQMNSLRPEDTALYYCTS | 13 |
| FR3, AbM] | | |
| 66B02_SO_CDR3 [CX3CR1, | DPRRGWDTRY | 21 |
| CDR3, AbM] | | |
| 66B02_SO_FR4 [CX3CR1, | WGQGTLVTVSS | 15 |
| FR4, AbM] | | |
| 66B02_SO_(E1D) FR1 | DVQLVESGGGSVQPGGSLRLSCAAS | 29 |
| [CX3CR1, FR1, AbM] | | |
| 307_FR1 [CX3CR1, FR1, | DVQLVESGGGLVQPGGSLRLSCAAS | 141 |
| AbM] | | |
| 307_CDR1 [CX3CR1, CDR1, | GSIFSSTAMA | 10 |
| AbM] | | |
| 307_FR2 [CX3CR1, FR2, | WYRQAPGKRRDLVA | 18 |
| AbM] | | |
| 307_CDR2 [CX3CR1, CDR2, | AISTVGVTK | 142 |
| AbM] | | |
| 307_FR3 [CX3CR1, FR3, | YADSVKGRFTISRDNSKNTVYLQMNSLRPEDTAVYYCTS | 143 |
| AbM] | | |
| 307_CDR3 [CX3CR1, CDR3, | DPRRGWDTRY | 21 |
| AbM] | | |
| 307_FR4 [CX3CR1, FR4, | WGQGTLVTVSS | 15 |
| AbM] | | |
| 307 (D1E)_FR1 [CX3CR1, | EVQLVESGGGLVQPGGSLRLSCAAS | 144 |
| FR1, AbM] | | |
| Formula [CX3CR1, CDR2, | X₁IX₃TVGX₇TK containing the ambiguity residue X | 202 |
| AbM] | | |
| Formula [CX3CR1, CDR3, | DXRRGWDTRY containing the ambiguity residue X | 138 |
| AbM] | | |

TABLE A-3
Anti-CX3CR1 ISVD sequence parts: CDRs according to
Kabat numbering and frameworks.

| Name [further info] | Sequence | ID |
|---|---|---|
| 54A12_FR1 [CX3CR1, FR1, | EVQLVESGGGSVQAGESLRLSCAASGSIFS | 145 |
| Kabat] | | |
| 54A12_CDR1 [CX3CR1, | SNAMA | 146 |
| CDR1, Kabat] | | |
| 54A12_FR2 [CX3CR1, FR2, | WYRQAPGKQRDLVA | 11 |
| Kabat] | | |
| 54A12_CDR2 [CX3CR1, | VINSVGITKYADSVKG | 147 |
| CDR2, Kabat] | | |
| 54A12_FR3 [CX3CR1, FR3, | RFTISGDNAKNTVYLQMNSLKPEDTAVYYCTS | 148 |
| Kabat] | | |
| 54A12_CDR3 [CX3CR1, | DARRGWDTRY | 14 |
| CDR3, Kabat] | | |
| 54A12_FR4 [CX3CR1, FR4, | WGQGTQVTVSS | 28 |
| Kabat] | | |
| 54A12_SO_FR1 [CX3CR1, | EVQLVESGGGSVQPGGSLRLSCAASGSIFS | 23 |
| FR1, Kabat] | | |
| 54A12_SO_CDR1 [CX3CR1, | STAMA | 24 |
| CDR1, Kabat] | | |
| 54A12_SO_FR2 [CX3CR1, | WYRQAPGKQRDLVA | 11 |
| FR2, Kabat] | | |
| 54A12_SO_CDR2 [CX3CR1, | VISTVGITKYADSVKG | 26 |
| CDR2, Kabat] | | |
| 54A12_SO_FR3 [CX3CR1, | RFTISRDNAKNTVYLQMNSLRPEDTALYYCTS | 27 |
| FR3, Kabat] | | |
| 54A12_SO_CDR3 [CX3CR1, | DARRGWDTRY | 14 |
| CDR3, Kabat] | | |
| 54A12_SO_FR4 [CX3CR1, | WGQGTLVTVSS | 15 |
| FR4, Kabat] | | |
| 54A12_SO (E1D)_FR1 | DVQLVESGGGSVQPGGSLRLSCAASGSIFS | 149 |
| [CX3CR1, FR1, Kabat] | | |
| 66B02_FR1 [CX3CR1, FR1, | EVQLVESGGGSVQAGESLRLSCAASGSIFS | 145 |
| Kabat] | | |
| 66B02_CDR1 [CX3CR1, | SNAMA | 146 |
| CDR1, Kabat] | | |
| 66B02_FR2 [CX3CR1, FR2, | WYRQAPGKRRDLVA | 18 |
| Kabat] | | |
| 66B02_CDR2 [CX3CR1, | AINSVGVTKYADSVKG | 150 |
| CDR2, Kabat] | | |
| 66B02_FR3 [CX3CR1, FR3, | RFTISRDNAKNTVYLQMNSLKPEDTAVYYCTS | 151 |
| Kabat] | | |
| 66B02_CDR3 [CX3CR1, | DPRRGWDTRY | 21 |
| CDR3, Kabat] | | |
| 66B02_FR4 [CX3CR1, FR4, | WGQGTQVTVSS | 28 |
| Kabat] | | |
| 66B02_SO_FR1 [CX3CR1, | EVQLVESGGGSVQPGGSLRLSCAASGSIFS | 23 |
| FR1, Kabat] | | |
| 66B02_SO_CDR1 [CX3CR1, | STAMA | 24 |
| CDR1, Kabat] | | |
| 66B02_SO_FR2 [CX3CR1, | WYRQAPGKRRDLVA | 18 |
| FR2, Kabat] | | |
| 66B02_SO_CDR2 [CX3CR1, | AINTVGVTKYADSVKG | 33 |
| CDR2, Kabat] | | |
| 66B02_SO_FR3 [CX3CR1, | RFTISRDNAKNTVYLQMNSLRPEDTALYYCTS | 27 |
| FR3, Kabat] | | |
| 66B02_SO_CDR3 [CX3CR1, | DPRRGWDTRY | 21 |
| CDR3, Kabat] | | |
| 66B02_SO_FR4 [CX3CR1, | WGQGTLVTVSS | 15 |
| FR4, Kabat] | | |
| 66B02_SO_(E1D) FR1 | DVQLVESGGGSVQPGGSLRLSCAASGSIFS | 149 |
| [CX3CR1, FR1, Kabat] | | |
| 307_FR1 [CX3CR1, FR1, | DVQLVESGGGLVQPGGSLRLSCAASGSIFS | 152 |
| Kabat] | | |
| 307_CDR1 [CX3CR1, CDR1, | STAMA | 24 |
| Kabat] | | |
| 307_FR2 [CX3CR1, FR2, | WYRQAPGKRRDLVA | 18 |
| Kabat] | | |
| 307_CDR2 [CX3CR1, CDR2, | AISTVGVTKYADSVKG | 64 |
| Kabat] | | |
| 307_FR3 [CX3CR1, FR3, | RFTISRDNSKNTVYLQMNSLRPEDTAVYYCTS | 65 |
| Kabat] | | |
| 307_CDR3 [CX3CR1, CDR3, | DPRRGWDTRY | 21 |
| Kabat] | | |
| 307_FR4 [CX3CR1, FR4, | WGQGTLVTVSS | 15 |
| Kabat] | | |
| 307_(D1E) FR1 [CX3CR1, | EVQLVESGGGLVQPGGSLRLSCAASGSIFS | 61 |
| FR1, Kabat] | | |

TABLE A-4
Serum albumin binding ISVD sequences.

| Name | | |
|---|---|---|
| [further | | |
| info] | Sequence | ID |
| ALB23002 | EVGLVESGGGVVQPGGSLRLSCAASGFTFRSFGMSWVRQAPGKGPEWVSSIS | 37 |
| | GSGSDTLYADSVKGRFTISRDNSKNTLYLQMNSLRPEDTALYYCTIGGSLSR | |
| | SSQGTLVTVSS | |
| Alb8 | EVGLVESGGGLVQPGNSLRLSCAASGFTFSSFGMSWVRQAPGKGLEWVSSIS | 38 |
| | GSGSDTLYADSVKGRFTISRDNAKTTLYLQMNSLRPEDTAVYYCTIGGSLSR | |
| | SSQGTLVTVSS | |
| Alb23 | EVQLLESGGGLVQPGGSLRLSCAASGFTFRSFGMSWVRQAPGKGPEWVSSIS | 39 |
| | GSGSDTLYADSVKGRFTISRDNSKNTLYLQMNSLRPEDTAVYYCTIGGSLSR | |
| | SSQGTLVTVSS | |
| Alb129 | EVQLVESGGGVVQPGNSLRLSCAASGFTFSSFGMSWVRQAPGKGLEWVSSIS | 40 |
| | GSGSDTLYADSVKGRFTISRDNAKTTLYLQMNSLRPEDTATYYCTIGGSLSR | |
| | SSQGTLVTVSSA | |
| Alb132 | EVGLVESGGGVVQPGGSLRLSCAASGFTFRSFGMSWVRQAPGKGPEWVSSIS | 41 |
| | GSGSDTLYADSVKGRFTISRDNSKNTLYLQMNSLRPEDTATYYCTIGGSLSR | |
| | SSQGTLVTVSSA | |
| Alb11 | EVGLVESGGGLVQPGNSLRLSCAASGFTFSSFGMSWVRQAPGKGLEWVSSIS | 42 |
| | GSGSDTLYADSVKGRFTISRDNAKTTLYLQMNSLRPEDTAVYYCTIGGSLSR | |
| | SSQGTLVTVSS | |
| Alb11 | EVGLVESGGGLVQPGNSLRLSCAASGFTFSSFGMSWVRQAPGKGLEWVSSIS | 43 |
| (S112K)-A | GSGSDTLYADSVKGRFTISRDNAKTTLYLQMNSLRPEDTAVYYCTIGGSLSR | |
| | SSQGTLVKVSSA | |
| Alb82 | EVGLVESGGGVVQPGNSLRLSCAASGFTFSSFGMSWVRQAPGKGLEWVSSIS | 44 |
| | GSGSDTLYADSVKGRFTISRDNAKTTLYLQMNSLRPEDTALYYCTIGGSLSR | |
| | SSQGTLVTVSS | |
| Alb82-A | EVGLVESGGGVVQPGNSLRLSCAASGFTFSSFGMSWVRQAPGKGLEWVSSIS | 45 |
| | GSGSDTLYADSVKGRFTISRDNAKTTLYLQMNSLRPEDTALYYCTIGGSLSR | |
| | SSQGTLVTVSSA | |
| Alb82-AA | EVGLVESGGGVVQPGNSLRLSCAASGFTFSSFGMSWVRQAPGKGLEWVSSIS | 46 |
| | GSGSDTLYADSVKGRFTISRDNAKTTLYLQMNSLRPEDTALYYCTIGGSLSR | |
| | SSQGTLVTVSSAA | |
| Alb82-AAA | EVGLVESGGGVVQPGNSLRLSCAASGFTFSSFGMSWVRQAPGKGLEWVSSIS | 47 |
| | GSGSDTLYADSVKGRFTISRDNAKTTLYLQMNSLRPEDTALYYCTIGGSLSR | |
| | SSQGTLVTVSSAAA | |
| Alb82-G | EVGLVESGGGVVQPGNSLRLSCAASGFTFSSFGMSWVRQAPGKGLEWVSSIS | 48 |
| | GSGSDTLYADSVKGRFTISRDNAKTTLYLQMNSLRPEDTALYYCTIGGSLSR | |
| | SSQGTLVTVSSG | |
| Alb82-GG | EVGLVESGGGVVQPGNSLRLSCAASGFTFSSFGMSWVRQAPGKGLEWVSSIS | 49 |
| | GSGSDTLYADSVKGRFTISRDNAKTTLYLQMNSLRPEDTALYYCTIGGSLSR | |
| | SSQGTLVTVSSGG | |
| Alb82-GGG | EVGLVESGGGVVQPGNSLRLSCAASGFTFSSFGMSWVRQAPGKGLEWVSSIS | 50 |
| | GSGSDTLYADSVKGRFTISRDNAKTTLYLQMNSLRPEDTALYYCTIGGSLSR | |
| | SSQGTLVTVSSGGG | |
| Alb223 | EVGLVESGGGVVQPGGSLRLSCAASGFTFRSFGMSWVRQAPGKGPEWVSSIS | 51 |
| | GSGSDTLYADSVKGRFTISRDNSKNTLYLQMNSLRPEDTALYYCTIGGSLSR | |
| | SSQGTLVTVSSA | |

TABLE A-5
Anti-albumin ISVD sequence parts: CDRs according
to AbM numbering and frameworks.

| Name [further info] | Sequence | ID |
|---|---|---|
| ALB23002_FR1 [albumin, | EVQLVESGGGVVQPGGSLRLSCAAS | 52 |
| FR1, AbM] | | |
| ALB23002_CDR1 [albumin, | GFTFRSFGMS | 53 |
| CDR1, AbM] | | |
| ALB23002_FR2 [albumin, | WVRQAPGKGPEWVS | 54 |
| FR2, AbM] | | |
| ALB23002_CDR2 [albumin, | SISGSGSDTL | 55 |
| CDR2, AbM] | | |
| ALB23002_FR3 [albumin, | YADSVKGRFTISRDNSKNTLYLQMN | 56 |
| FR3, AbM] | SLRPEDTALYYCTI | |
| ALB23002_CDR3 [albumin, | GGSLSR | 57 |
| CDR3, AbM] | | |
| ALB23002_FR4 [albumin, | SSQGTLVTVSS | 58 |
| FR4, AbM] | | |

TABLE A-6
Anti-albumin ISVD sequence parts: CDRs according
to Kabat numbering and frameworks.

| Name [further info] | Sequence | ID |
|---|---|---|
| ALB23002_FR1 [albumin, | EVQLVESGGGVVQPGGSLRLSCAAS | 59 |
| FR1, Kabat] | GFTER | |
| ALB23002_CDR1 [albumin, | SFGMS | 60 |
| CDR1, Kabat] | | |
| ALB23002_FR2 [albumin, | WVRQAPGKGPEWVS | 54 |
| FR2, Kabat] | | |
| ALB23002_CDR2 [albumin, | SISGSGSDTLYADSVKG | 62 |
| CDR2, Kabat] | | |
| ALB23002_FR3 [albumin, | RFTISRDNSKNTLYLQMNSLRPEDT | 63 |
| FR3, Kabat] | ALYYCTI | |
| ALB23002_CDR3 [albumin, | GGSLSR | 57 |
| CDR3, Kabat] | | |
| ALB23002_FR4 [albumin, | SSQGTLVTVSS | 58 |
| FR4, Kabat] | | |

TABLE A-7
Peptidic linker sequences.

| Name | | |
|---|---|---|
| [further | | |
| info] | Sequence | ID |
| 3A linker | AAA | — |
| 5GS linker | GGGGS | 66 |
| 7GS linker | SGGSGGS | 67 |
| 8GS linker | GGGGSGGS | 68 |
| 9GS linker | GGGGSGGGS | 69 |
| 10GS linker | GGGGSGGGGS | 70 |
| 15GS linker | GGGGSGGGGSGGGGS | 71 |
| 18GS linker | GGGGSGGGGSGGGGSGGS | 72 |
| 20GS linker | GGGGSGGGGSGGGGSGGGGS | 73 |
| 25GS linker | GGGGSGGGGSGGGGSGGGGSGGGGS | 74 |
| 30GS linker | GGGGGGGGSGGGGSGGGGSGGGGSGGGGS | 75 |
| 35GS linker | GGGGSGGGGSGGGGSGGGGSGGGGSGGGG | 76 |
| | SGGGGS | |
| 40GS linker | GGGGSGGGGSGGGGSGGGGSGGGGSGGGG | 77 |
| | SGGGGSGGGGS | |
| G1 hinge | EPKSCDKTHTCPPCP | 78 |
| 9GS-G1 hinge | GGGGSGGGSEPKSCDKTHTCPPCP | 79 |
| Llama upper | EPKTPKPQPAAA | 80 |
| long hinge | | |
| region | | |
| G3 hinge | ELKTPLGDTTHTCPRCPEPKSCDTPPPCP | 81 |
| | RCPEPKSCDTPPPCPRCPEPKSCDTPPPC | |
| | PRCP | |
| (gly3ser2)3 | GGGSSGGGSSGGGSS | 136 |
| linker | | |

TABLE A-8
Exemplary multivalent ISVD polypeptides.

| Name | | |
|---|---|---|
| [further | | |
| info] | Sequence | ID |
| A041600010 | DVQLVESGGGSVQPGGSLRLSCAASGSIFSSTAMAWYRQAPGKRRDLVAAIN | 153 |
| | TVGVTKYADSVKGRFTISRDNAKNTVYLQMNSLRPEDTALYYCTSDPRRGWD | |
| | TRYWGQGTLVTVSSGGGGSGGGSEVQLVESGGGVVQPGGSLRLSCAASGFTF | |
| | RSFGMSWVRQAPGKGPEWVSSISGSGSDTLYADSVKGRFTISRDNSKNTLYL | |
| | QMNSLRPEDTALYYCTIGGSLSRSSQGTLVTVSSGGGGSGGGSEVQLVESGG | |
| | GSVQPGGSLRLSCAASGSIFSSTAMAWYRQAPGKQRDLVAVISTVGITKYAD | |
| | SVKGRFTISRDNAKNTVYLQMNSLRPEDTALYYCTSDARRGWDTRYWGQGTL | |
| | VTVSSA | |
| A041600011 | DVQLVESGGGSVQPGGSLRLSCAASGSIFSSTAMAWYRQAPGKRRDLVAAIN | 154 |
| | TVGVTKYADSVKGRFTISRDNAKNTVYLQMNSLRPEDTALYYCTSDPRRGWD | |
| | TRYWGQGTLVTVSSGGGGSGGGSEVQLVESGGGSVQPGGSLRLSCAASGSIF | |
| | SSTAMAWYRQAPGKRRDLVAAINTVGVTKYADSVKGRFTISRDNAKNTVYLQ | |
| | MNSLRPEDTALYYCTSDPRRGWDTRYWGQGTLVTVSSGGGGSGGGSEVQLVE | |
| | SGGGVVQPGGSLRLSCAASGFTFRSFGMSWVRQAPGKGPEWVSSISGSGSDT | |
| | LYADSVKGRFTISRDNSKNTLYLQMNSLRPEDTALYYCTIGGSLSRSSQGTL | |
| | VTVSSA | |
| A041600025 | DVQLVESGGGSVQPGGSLRLSCAASGSIFSSTAMAWYRQAPGKQRDLVAVIS | 155 |
| | TVGITKYADSVKGRFTISRDNAKNTVYLQMNSLRPEDTALYYCTSDARRGWD | |
| | TRYWGQGTLVTVSSGGGGSGGGSEVQLVESGGGVVQPGGSLRLSCAASGFTF | |
| | RSFGMSWVRQAPGKGPEWVSSISGSGSDTLYADSVKGRFTISRDNSKNTLYL | |
| | QMNSLRPEDTALYYCTIGGSLSRSSQGTLVTVSSGGGGSGGGSEVQLVESGG | |
| | GSVQPGGSLRLSCAASGSIFSSTAMAWYRQAPGKQRDLVAVISTVGITKYAD | |
| | SVKGRFTISRDNAKNTVYLQMNSLRPEDTALYYCTSDARRGWDTRYWGQGTL | |
| | VTVSSA | |
| A041600026 | DVQLVESGGGSVQPGGSLRLSCAASGSIFSSTAMAWYRQAPGKRRDLVAAIN | 156 |
| | TVGVTKYADSVKGRFTISRDNAKNTVYLQMNSLRPEDTALYYCTSDPRRGWD | |
| | TRYWGQGTLVTVSSGGGGSGGGGSGGGGSGGGGSEVQLVESGGGVVQPGGSL | |
| | RLSCAASGFTFRSFGMSWVRQAPGKGPEWVSSISGSGSDTLYADSVKGRFTI | |
| | SRDNSKNTLYLQMNSLRPEDTALYYCTIGGSLSRSSQGTLVTVSSGGGGSGG | |
| | GGSGGGGSGGGGSEVQLVESGGGSVQPGGSLRLSCAASGSIFSSTAMAWYRQ | |
| | APGKQRDLVAVISTVGITKYADSVKGRFTISRDNAKNTVYLQMNSLRPEDTA | |
| | LYYCTSDARRGWDTRYWGQGTLVTVSSA | |
| A041600027 | DVQLVESGGGSVQPGGSLRLSCAASGSIFSSTAMAWYRQAPGKRRDLVAAIN | 157 |
| | TVGVTKYADSVKGRFTISRDNAKNTVYLQMNSLRPEDTALYYCTSDPRRGWD | |
| | TRYWGQGTLVTVSSGGGGSGGGSEVQLVESGGGVVQPGGSLRLSCAASGFTF | |
| | RSFGMSWVRQAPGKGPEWVSSISGSGSDTLYADSVKGRFTISRDNSKNTLYL | |
| | QMNSLRPEDTALYYCTIGGSLSRSSQGTLVTVSSGGGGSGGGGSGGGGSGGG | |
| | GSEVQLVESGGGSVQPGGSLRLSCAASGSIFSSTAMAWYRQAPGKQRDLVAV | |
| | ISTVGITKYADSVKGRFTISRDNAKNTVYLQMNSLRPEDTALYYCTSDARRG | |
| | WDTRYWGQGTLVTVSSA | |
| A041600028 | DVQLVESGGGSVQPGGSLRLSCAASGSIFSSTAMAWYRQAPGKRRDLVAAIN | 158 |
| | TVGVTKYADSVKGRFTISRDNAKNTVYLQMNSLRPEDTALYYCTSDPRRGWD | |
| | TRYWGQGTLVTVSSGGGGSGGGSEVQLVESGGGVVQPGGSLRLSCAASGFTF | |
| | RSFGMSWVRQAPGKGPEWVSSISGSGSDTLYADSVKGRFTISRDNSKNTLYL | |
| | QMNSLRPEDTALYYCTIGGSLSRSSQGTLVTVSSGGGGSGGGGSGGGGSGGG | |
| | GSGGGGSGGGGSGGGGSEVQLVESGGGSVQPGGSLRLSCAASGSIFSSTAMA | |
| | WYRQAPGKQRDLVAVISTVGITKYADSVKGRFTISRDNAKNTVYLQMNSLRP | |
| | EDTALYYCTSDARRGWDTRYWGQGTLVTVSSA | |
| A041600029 | DVQLVESGGGSVQPGGSLRLSCAASGSIFSSTAMAWYRQAPGKRRDLVAAIN | 159 |
| | TVGVTKYADSVKGRFTISRDNAKNTVYLQMNSLRPEDTALYYCTSDPRRGWD | |
| | TRYWGQGTLVTVSSGGGGSGGGGSGGGGSGGGGSEVQLVESGGGVVQPGGSL | |
| | RLSCAASGFTFRSFGMSWVRQAPGKGPEWVSSISGSGSDTLYADSVKGRFTI | |
| | SRDNSKNTLYLQMNSLRPEDTALYYCTIGGSLSRSSQGTLVTVSSGGGGSGG | |
| | GSEVQLVESGGGSVQPGGSLRLSCAASGSIFSSTAMAWYRQAPGKQRDLVAV | |
| | ISTVGITKYADSVKGRFTISRDNAKNTVYLQMNSLRPEDTALYYCTSDARRG | |
| | WDTRYWGQGTLVTVSSA | |
| A041600030 | DVQLVESGGGSVQPGGSLRLSCAASGSIFSSTAMAWYRQAPGKRRDLVAAIN | 160 |
| | TVGVTKYADSVKGRFTISRDNAKNTVYLQMNSLRPEDTALYYCTSDPRRGWD | |
| | TRYWGQGTLVTVSSGGGGSGGGGSGGGGSGGGGSEVQLVESGGGVVQPGGSL | |
| | RLSCAASGFTFRSFGMSWVRQAPGKGPEWVSSISGSGSDTLYADSVKGRFTI | |
| | SRDNSKNTLYLQMNSLRPEDTALYYCTIGGSLSRSSQGTLVTVSSGGGGSGG | |
| | GGSGGGGSGGGGSGGGGSGGGGSGGGGSEVQLVESGGGSVQPGGSLRLSCAA | |
| | SGSIFSSTAMAWYRQAPGKQRDLVAVISTVGITKYADSVKGRFTISRDNAKN | |
| | TVYLQMNSLRPEDTALYYCTSDARRGWDTRYWGQGTLVTVSSA | |
| A041600031 | DVQLVESGGGSVQPGGSLRLSCAASGSIFSSTAMAWYRQAPGKRRDLVAAIN | 161 |
| | TVGVTKYADSVKGRFTISRDNAKNTVYLQMNSLRPEDTALYYCTSDPRRGWD | |
| | TRYWGQGTLVTVSSGGGGSGGGSEVQLVESGGGVVQPGGSLRLSCAASGFTF | |
| | RSFGMSWVRQAPGKGPEWVSSISGSGSDTLYADSVKGRFTISRDNSKNTLYL | |
| | QMNSLRPEDTALYYCTIGGSLSRSSQGTLVTVSSGGGGSGGGSEVQLVESGG | |
| | GSVQPGGSLRLSCAASGSIFSSTAMAWYRQAPGKQRDLVAVISTVGITKYAD | |
| | SVKGRFTISRDNAKNTVYLQMNSLRPEDTALYYCTSDARRGWDTRYWGQGTL | |
| | VTVSSA | |
| A041600032 | DVQLVESGGGSVQPGGSLRLSCAASGSIFSSTAMAWYRQAPGKRRDLVAAIN | 162 |
| | TVGVTKYADSVKGRFTISRDNAKNTVYLQMNSLRPEDTALYYCTSDPRRGWD | |
| | TRYWGQGTLVTVSSGGGGSGGGGSGGGGSGGGGSGGGGSGGGGSGGGGSEVQ | |
| | LVESGGGVVQPGGSLRLSCAASGFTFRSFGMSWVRQAPGKGPEWVSSISGSG | |
| | SDTLYADSVKGRFTISRDNSKNTLYLQMNSLRPEDTALYYCTIGGSLSRSSQ | |
| | GTLVTVSSGGGGSGGGSEVQLVESGGGSVQPGGSLRLSCAASGSIFSSTAMA | |
| | WYRQAPGKQRDLVAVISTVGITKYADSVKGRFTISRDNAKNTVYLQMNSLRP | |
| | EDTALYYCTSDARRGWDTRYWGQGTLVTVSSA | |
| A041600033 | DVQLVESGGGSVQPGGSLRLSCAASGSIFSSTAMAWYRQAPGKRRDLVAAIN | 163 |
| | TVGVTKYADSVKGRFTISRDNAKNTVYLQMNSLRPEDTALYYCTSDPRRGWD | |
| | TRYWGQGTLVTVSSGGGGSGGGGSGGGGSGGGGSGGGGSGGGGSGGGGSEVQ | |
| | LVESGGGVVQPGGSLRLSCAASGFTFRSFGMSWVRQAPGKGPEWVSSISGSG | |
| | SDTLYADSVKGRFTISRDNSKNTLYLQMNSLRPEDTALYYCTIGGSLSRSSQ | |
| | GTLVTVSSGGGGSGGGGSGGGGSGGGGSEVQLVESGGGSVQPGGSLRLSCAA | |
| | SGSIFSSTAMAWYRQAPGKQRDLVAVISTVGITKYADSVKGRFTISRDNAKN | |
| | TVYLQMNSLRPEDTALYYCTSDARRGWDTRYWGQGTLVTVSSA | |
| A041600034 | DVQLVESGGGSVQPGGSLRLSCAASGSIFSSTAMAWYRQAPGKQRDLVAVIS | 164 |
| | TVGITKYADSVKGRFTISRDNAKNTVYLQMNSLRPEDTALYYCTSDARRGWD | |
| | TRYWGQGTLVTVSSGGGGSGGGSEVQLVESGGGVVQPGGSLRLSCAASGFTF | |
| | RSFGMSWVRQAPGKGPEWVSSISGSGSDTLYADSVKGRFTISRDNSKNTLYL | |
| | QMNSLRPEDTALYYCTIGGSLSRSSQGTLVTVSSGGGGSGGGSEVQLVESGG | |
| | GSVQPGGSLRLSCAASGSIFSSTAMAWYRQAPGKQRDLVAVISTVGITKYAD | |
| | SVKGRFTISRDNAKNTVYLQMNSLRPEDTALYYCTSDARRGWDTRYWGQGTL | |
| | VTVSSA | |
| A041600035 | DVQLVESGGGSVQPGGSLRLSCAASGSIFSSTAMAWYRQAPGKQRDLVAVIS | 82 |
| | TVGITKYADSVKGRFTISRDNAKNTVYLQMNSLRPEDTALYYCTSDARRGWD | |
| | TRYWGQGTLVTVSSGGGGSGGGSEVQLVESGGGSVQPGGSLRLSCAASGSIF | |
| | SSTAMAWYRQAPGKQRDLVAVISTVGITKYADSVKGRFTISRDNAKNTVYLQ | |
| | MNSLRPEDTALYYCTSDARRGWDTRYWGQGTLVTVSSGGGGSGGGSEVQLVE | |
| | SGGGVVQPGGSLRLSCAASGFTERSFGMSWVRQAPGKGPEWVSSISGSGSDT | |
| | LYADSVKGRFTISRDNSKNTLYLQMNSLRPEDTALYYCTIGGSLSRSSQGTL | |
| | VTVSSA | |
| A041600036 | DVQLVESGGGSVQPGGSLRLSCAASGSIFSSTAMAWYRQAPGKRRDLVAAIN | 165 |
| | TVGVTKYADSVKGRFTISRDNAKNTVYLQMNSLRPEDTALYYCTSDPRRGWD | |
| | TRYWGQGTLVTVSSGGGGSGGGGSGGGGSGGGGSEVQLVESGGGSVQPGGSL | |
| | RLSCAASGSIFSSTAMAWYRQAPGKRRDLVAAINTVGVTKYADSVKGRFTIS | |
| | RDNAKNTVYLQMNSLRPEDTALYYCTSDPRRGWDTRYWGQGTLVTVSSGGGG | |
| | SGGGSEVQLVESGGGVVQPGGSLRLSCAASGFTFRSFGMSWVRQAPGKGPEW | |
| | VSSISGSGSDTLYADSVKGRFTISRDNSKNTLYLQMNSLRPEDTALYYCTIG | |
| | GSLSRSSQGTLVTVSSA | |
| A041600037 | DVQLVESGGGSVQPGGSLRLSCAASGSIFSSTAMAWYRQAPGKRRDLVAAIN | 166 |
| | TVGVTKYADSVKGRFTISRDNAKNTVYLQMNSLRPEDTALYYCTSDPRRGWD | |
| | TRYWGQGTLVTVSSGGGGSGGGGSGGGGSGGGGSEVQLVESGGGSVQPGGSL | |
| | RLSCAASGSIFSSTAMAWYRQAPGKRRDLVAAINTVGVTKYADSVKGRFTIS | |
| | RDNAKNTVYLQMNSLRPEDTALYYCTSDPRRGWDTRYWGQGTLVTVSSGGGG | |
| | SGGGGSGGGGSGGGGSGGGGSGGGGSGGGGSEVQLVESGGGVVQPGGSLRLS | |
| | CAASGFTFRSFGMSWVRQAPGKGPEWVSSISGSGSDTLYADSVKGRFTISRD | |
| | NSKNTLYLQMNSLRPEDTALYYCTIGGSLSRSSQGTLVTVSSA | |
| A041600038 | DVQLVESGGGSVQPGGSLRLSCAASGSIFSSTAMAWYRQAPGKRRDLVAAIN | 167 |
| | TVGVTKYADSVKGRFTISRDNAKNTVYLQMNSLRPEDTALYYCTSDPRRGWD | |
| | TRYWGQGTLVTVSSGGGGGGGGSGGGGSGGGGSGGGGSGGGGSGGGGSEVQL | |
| | VESGGGSVQPGGSLRLSCAASGSIFSSTAMAWYRQAPGKRRDLVAAINTVGV | |
| | TKYADSVKGRFTISRDNAKNTVYLQMNSLRPEDTALYYCTSDPRRGWDTRYW | |
| | GQGTLVTVSSGGGGSGGGSEVQLVESGGGVVQPGGSLRLSCAASGFTFRSFG | |
| | MSWVRQAPGKGPEWVSSISGSGSDTLYADSVKGRFTISRDNSKNTLYLQMNS | |
| | LRPEDTALYYCTIGGSLSRSSQGTLVTVSSA | |
| A041600039 | DVQLVESGGGSVQPGGSLRLSCAASGSIFSSTAMAWYRQAPGKRRDLVAAIN | 168 |
| | TVGVTKYADSVKGRFTISRDNAKNTVYLQMNSLRPEDTALYYCTSDPRRGWD | |
| | TRYWGQGTLVTVSSGGGGSGGGGSGGGGSGGGGSGGGGSGGGGSGGGGSEVQ | |
| | LVESGGGSVQPGGSLRLSCAASGSIFSSTAMAWYRQAPGKRRDLVAAINTVG | |
| | VTKYADSVKGRFTISRDNAKNTVYLQMNSLRPEDTALYYCTSDPRRGWDTRY | |
| | WGQGTLVTVSSGGGGSGGGGSGGGGSGGGGSEVQLVESGGGVVQPGGSLRLS | |
| | CAASGFTFRSFGMSWVRQAPGKGPEWVSSISGSGSDTLYADSVKGRFTISRD | |
| | NSKNTLYLQMNSLRPEDTALYYCTIGGSLSRSSQGTLVTVSSA | |
| A041600040 | DVQLVESGGGSVQPGGSLRLSCAASGSIFSSTAMAWYRQAPGKRRDLVAAIN | 169 |
| | TVGVTKYADSVKGRFTISRDNAKNTVYLQMNSLRPEDTALYYCTSDPRRGWD | |
| | TRYWGQGTLVTVSSGGGGSGGGSEVQLVESGGGSVQPGGSLRLSCAASGSIF | |
| | SSTAMAWYRQAPGKQRDLVAVISTVGITKYADSVKGRFTISRDNAKNTVYLQ | |
| | MNSLRPEDTALYYCTSDARRGWDTRYWGQGTLVTVSSGGGGSGGGSEVQLVE | |
| | SGGGVVQPGGSLRLSCAASGFTFRSFGMSWVRQAPGKGPEWVSSISGSGSDT | |
| | LYADSVKGRFTISRDNSKNTLYLQMNSLRPEDTALYYCTIGGSLSRSSQGTL | |
| | VTVSSA | |
| A041600041 | DVQLVESGGGSVQPGGSLRLSCAASGSIFSSTAMAWYRQAPGKQRDLVAVIS | 170 |
| | TVGITKYADSVKGRFTISRDNAKNTVYLQMNSLRPEDTALYYCTSDARRGWD | |
| | TRYWGQGTLVTVSSGGGGSGGGSEVQLVESGGGSVQPGGSLRLSCAASGSIF | |
| | SSTAMAWYRQAPGKRRDLVAAINTVGVTKYADSVKGRFTISRDNAKNTVYLQ | |
| | MNSLRPEDTALYYCTSDPRRGWDTRYWGQGTLVTVSSGGGGSGGGSEVQLVE | |
| | SGGGVVQPGGSLRLSCAASGFTFRSFGMSWVRQAPGKGPEWVSSISGSGSDT | |
| | LYADSVKGRFTISRDNSKNTLYLQMNSLRPEDTALYYCTIGGSLSRSSQGTL | |
| | VTVSSA | |
| A041600042 | DVQLVESGGGSVQPGGSLRLSCAASGSIFSSTAMAWYRQAPGKRRDLVAAIN | 171 |
| | TVGVTKYADSVKGRFTISRDNAKNTVYLQMNSLRPEDTALYYCTSDPRRGWD | |
| | TRYWGQGTLVTVSSGGGGSGGGGSGGGGSGGGGSEVQLVESGGGSVQPGGSL | |
| | RLSCAASGSIFSSTAMAWYRQAPGKRRDLVAAINTVGVTKYADSVKGRFTIS | |
| | RDNAKNTVYLQMNSLRPEDTALYYCTSDPRRGWDTRYWGQGTLVTVSSGGGG | |
| | SGGGGSGGGGSGGGGSEVQLVESGGGVVQPGGSLRLSCAASGFTFRSFGMSW | |
| | VRQAPGKGPEWVSSISGSGSDTLYADSVKGRFTISRDNSKNTLYLQMNSLRP | |
| | EDTALYYCTIGGSLSRSSQGTLVTVSSA | |
| A041600043 | DVQLVESGGGSVQPGGSLRLSCAASGSIFSSTAMAWYRQAPGKRRDLVAAIN | 172 |
| | TVGVTKYADSVKGRFTISRDNAKNTVYLQMNSLRPEDTALYYCTSDPRRGWD | |
| | TRYWGQGTLVTVSSGGGGSGGGGSGGGGSGGGGSGGGGGGGGSGGGGSEVQL | |
| | VESGGGSVQPGGSLRLSCAASGSIFSSTAMAWYRQAPGKRRDLVAAINTVGV | |
| | TKYADSVKGRFTISRDNAKNTVYLQMNSLRPEDTALYYCTSDPRRGWDTRYW | |
| | GQGTLVTVSSGGGGSGGGGSGGGGSGGGGSGGGGSGGGGSGGGGSEVQLVES | |
| | GGGVVQPGGSLRLSCAASGFTFRSFGMSWVRQAPGKGPEWVSSISGSGSDTL | |
| | YADSVKGRFTISRDNSKNTLYLQMNSLRPEDTALYYCTIGGSLSRSSQGTLV | |
| | TVSSA | |
| A041600044 | DVQLVESGGGSVQPGGSLRLSCAASGSIFSSTAMAWYRQAPGKRRDLVAAIN | 173 |
| | TVGVTKYADSVKGRFTISRDNAKNTVYLQMNSLRPEDTALYYCTSDPRRGWD | |
| | TRYWGQGTLVTVSSGGGGSGGGSEVQLVESGGGSVQPGGSLRLSCAASGSIF | |
| | SSTAMAWYRQAPGKRRDLVAAINTVGVTKYADSVKGRFTISRDNAKNTVYLQ | |
| | MNSLRPEDTALYYCTSDPRRGWDTRYWGQGTLVTVSSGGGGSGGGGSGGGGS | |
| | GGGGSEVQLVESGGGVVQPGGSLRLSCAASGFTFRSFGMSWVRQAPGKGPEW | |
| | VSSISGSGSDTLYADSVKGRFTISRDNSKNTLYLQMNSLRPEDTALYYCTIG | |
| | GSLSRSSQGTLVTVSSA | |
| A041600045 | DVQLVESGGGSVQPGGSLRLSCAASGSIFSSTAMAWYRQAPGKRRDLVAAIN | 174 |
| | TVGVTKYADSVKGRFTISRDNAKNTVYLQMNSLRPEDTALYYCTSDPRRGWD | |
| | TRYWGQGTLVTVSSGGGGSGGGSEVQLVESGGGSVQPGGSLRLSCAASGSIF | |
| | SSTAMAWYRQAPGKRRDLVAAINTVGVTKYADSVKGRFTISRDNAKNTVYLQ | |
| | MNSLRPEDTALYYCTSDPRRGWDTRYWGQGTLVTVSSGGGGSGGGGGGGGSG | |
| | GGGSGGGGSGGGGSGGGGSEVQLVESGGGVVQPGGSLRLSCAASGFTFRSFG | |
| | MSWVRQAPGKGPEWVSSISGSGSDTLYADSVKGRFTISRDNSKNTLYLQMNS | |
| | LRPEDTALYYCTIGGSLSRSSQGTLVTVSSA | |
| A041600046 | DVQLVESGGGSVQPGGSLRLSCAASGSIFSSTAMAWYRQAPGKRRDLVAAIN | 175 |
| | TVGVTKYADSVKGRFTISRDNAKNTVYLQMNSLRPEDTALYYCTSDPRRGWD | |
| | TRYWGQGTLVTVSSGGGGSGGGGSGGGGSGGGGSGGGGSGGGGSGGGGSEVQ | |
| | LVESGGGVVQPGGSLRLSCAASGFTFRSFGMSWVRQAPGKGPEWVSSISGSG | |
| | SDTLYADSVKGRFTISRDNSKNTLYLQMNSLRPEDTALYYCTIGGSLSRSSQ | |
| | GTLVTVSSGGGGSGGGGSGGGGGGGGSGGGGSGGGGSGGGGSEVQLVESGGG | |
| | SVQPGGSLRLSCAASGSIFSSTAMAWYRQAPGKQRDLVAVISTVGITKYADS | |
| | VKGRFTISRDNAKNTVYLQMNSLRPEDTALYYCTSDARRGWDTRYWGQGTLV | |
| | TVSSA | |
| A041600085 | DVQLVESGGGLVQPGGSLRLSCAASGSIFSSTAMAWYRQAPGKRRDLVAAIS | 137 |
| | TVGVTKYADSVKGRFTISRDNSKNTVYLQMNSLRPEDTAVYYCTSDPRRGWD | |
| | TRYWGQGTLVTVSSGGGGSGGGSEVQLVESGGGVVQPGGSLRLSCAASGFTF | |
| | RSFGMSWVRQAPGKGPEWVSSISGSGSDTLYADSVKGRFTISRDNSKNTLYL | |
| | QMNSLRPEDTALYYCTIGGSLSRSSQGTLVTVSSGGGGSGGGSEVQLVESGG | |
| | GLVQPGGSLRLSCAASGSIFSSTAMAWYRQAPGKRRDLVAAISTVGVTKYAD | |
| | SVKGRFTISRDNSKNTVYLQMNSLRPEDTAVYYCTSDPRRGWDTRYWGQGTL | |
| | VTVSSA | |
| A041600087 | DVQLVESGGGLVQPGGSLRLSCAASGSIFSSTAMAWYRQAPGKRRDLVAAIS | 83 |
| | TVGVTKYADSVKGRFTISRDNSKNTVYLQMNSLRPEDTAVYYCTSDPRRGWD | |
| | TRYWGQGTLVTVSSGGGGSGGGSEVQLVESGGGLVQPGNSLRLSCAASGFTF | |
| | SSFGMSWVRQAPGKGLEWVSSISGSGSDTLYADSVKGRFTISRDNAKTTLYL | |
| | QMNSLRPEDTAVYYCTIGGSLSRSSQGTLVTVSSGGGGSGGGSEVQLVESGG | |
| | GLVQPGGSLRLSCAASGSIFSSTAMAWYRQAPGKRRDLVAAISTVGVTKYAD | |
| | SVKGRFTISRDNSKNTVYLQMNSLRPEDTAVYYCTSDPRRGWDTRYWGQGTL | |
| | VTVSS | |
| A041600010 | EVQLVESGGGSVQPGGSLRLSCAASGSIFSSTAMAWYRQAPGKRRDLVAAIN | 176 |
| (D1E) | TVGVTKYADSVKGRFTISRDNAKNTVYLQMNSLRPEDTALYYCTSDPRRGWD | |
| | TRYWGQGTLVTVSSGGGGSGGGSEVQLVESGGGVVQPGGSLRLSCAASGFTF | |
| | RSFGMSWVRQAPGKGPEWVSSISGSGSDTLYADSVKGRFTISRDNSKNTLYL | |
| | QMNSLRPEDTALYYCTIGGSLSRSSQGTLVTVSSGGGGSGGGSEVQLVESGG | |
| | GSVQPGGSLRLSCAASGSIFSSTAMAWYRQAPGKQRDLVAVISTVGITKYAD | |
| | SVKGRFTISRDNAKNTVYLQMNSLRPEDTALYYCTSDARRGWDTRYWGQGTL | |
| | VTVSSA | |
| A041600011 | EVQLVESGGGSVQPGGSLRLSCAASGSIFSSTAMAWYRQAPGKRRDLVAAIN | 177 |
| (D1E) | TVGVTKYADSVKGRFTISRDNAKNTVYLQMNSLRPEDTALYYCTSDPRRGWD | |
| | TRYWGQGTLVTVSSGGGGSGGGSEVQLVESGGGSVQPGGSLRLSCAASGSIF | |
| | SSTAMAWYRQAPGKRRDLVAAINTVGVTKYADSVKGRFTISRDNAKNTVYLQ | |
| | MNSLRPEDTALYYCTSDPRRGWDTRYWGQGTLVTVSSGGGGSGGGSEVQLVE | |
| | SGGGVVQPGGSLRLSCAASGFTFRSFGMSWVRQAPGKGPEWVSSISGSGSDT | |
| | LYADSVKGRFTISRDNSKNTLYLQMNSLRPEDTALYYCTIGGSLSRSSQGTL | |
| | VTVSSA | |
| A041600025 | EVQLVESGGGSVQPGGSLRLSCAASGSIFSSTAMAWYRQAPGKQRDLVAVIS | 178 |
| (D1E) | TVGITKYADSVKGRFTISRDNAKNTVYLQMNSLRPEDTALYYCTSDARRGWD | |
| | TRYWGQGTLVTVSSGGGGSGGGSEVQLVESGGGVVQPGGSLRLSCAASGFTE | |
| | RSFGMSWVRQAPGKGPEWVSSISGSGSDTLYADSVKGRFTISRDNSKNTLYL | |
| | QMNSLRPEDTALYYCTIGGSLSRSSQGTLVTVSSGGGGSGGGSEVQLVESGG | |
| | GSVQPGGSLRLSCAASGSIFSSTAMAWYRQAPGKQRDLVAVISTVGITKYAD | |
| | SVKGRFTISRDNAKNTVYLQMNSLRPEDTALYYCTSDARRGWDTRYWGQGTL | |
| | VTVSSA | |
| A041600026 | EVQLVESGGGSVQPGGSLRLSCAASGSIFSSTAMAWYRQAPGKRRDLVAAIN | 179 |
| (D1E) | TVGVTKYADSVKGRFTISRDNAKNTVYLQMNSLRPEDTALYYCTSDPRRGWD | |
| | TRYWGQGTLVTVSSGGGGSGGGGSGGGGSGGGGSEVQLVESGGGVVQPGGSL | |
| | RLSCAASGFTFRSFGMSWVRQAPGKGPEWVSSISGSGSDTLYADSVKGRFTI | |
| | SRDNSKNTLYLQMNSLRPEDTALYYCTIGGSLSRSSQGTLVTVSSGGGGSGG | |
| | GGSGGGGSGGGGSEVQLVESGGGSVQPGGSLRLSCAASGSIFSSTAMAWYRQ | |
| | APGKQRDLVAVISTVGITKYADSVKGRFTISRDNAKNTVYLQMNSLRPEDTA | |
| | LYYCTSDARRGWDTRYWGQGTLVTVSSA | |
| A041600027 | EVQLVESGGGSVQPGGSLRLSCAASGSIFSSTAMAWYRQAPGKRRDLVAAIN | 180 |
| (D1E) | TVGVTKYADSVKGRFTISRDNAKNTVYLQMNSLRPEDTALYYCTSDPRRGWD | |
| | TRYWGQGTLVTVSSGGGGSGGGSEVQLVESGGGVVQPGGSLRLSCAASGFTF | |
| | RSFGMSWVRQAPGKGPEWVSSISGSGSDTLYADSVKGRFTISRDNSKNTLYL | |
| | QMNSLRPEDTALYYCTIGGSLSRSSQGTLVTVSSGGGGSGGGGSGGGGSGGG | |
| | GSEVQLVESGGGSVQPGGSLRLSCAASGSIFSSTAMAWYRQAPGKQRDLVAV | |
| | ISTVGITKYADSVKGRFTISRDNAKNTVYLQMNSLRPEDTALYYCTSDARRG | |
| | WDTRYWGQGTLVTVSSA | |
| A041600028 | EVQLVESGGGSVQPGGSLRLSCAASGSIFSSTAMAWYRQAPGKRRDLVAAIN | 181 |
| (D1E) | TVGVTKYADSVKGRFTISRDNAKNTVYLQMNSLRPEDTALYYCTSDPRRGWD | |
| | TRYWGQGTLVTVSSGGGGSGGGSEVQLVESGGGVVQPGGSLRLSCAASGFTF | |
| | RSFGMSWVRQAPGKGPEWVSSISGSGSDTLYADSVKGRFTISRDNSKNTLYL | |
| | QMNSLRPEDTALYYCTIGGSLSRSSQGTLVTVSSGGGGSGGGGSGGGGSGGG | |
| | GSGGGGSGGGGSGGGGSEVQLVESGGGSVQPGGSLRLSCAASGSIFSSTAMA | |
| | WYRQAPGKQRDLVAVISTVGITKYADSVKGRFTISRDNAKNTVYLQMNSLRP | |
| | EDTALYYCTSDARRGWDTRYWGQGTLVTVSSA | |
| A041600029 | EVQLVESGGGSVQPGGSLRLSCAASGSIFSSTAMAWYRQAPGKRRDLVAAIN | 182 |
| (D1E) | TVGVTKYADSVKGRFTISRDNAKNTVYLQMNSLRPEDTALYYCTSDPRRGWD | |
| | TRYWGQGTLVTVSSGGGGSGGGGSGGGGSGGGGSEVQLVESGGGVVQPGGSL | |
| | RLSCAASGFTFRSFGMSWVRQAPGKGPEWVSSISGSGSDTLYADSVKGRFTI | |
| | SRDNSKNTLYLQMNSLRPEDTALYYCTIGGSLSRSSQGTLVTVSSGGGGSGG | |
| | GSEVQLVESGGGSVQPGGSLRLSCAASGSIFSSTAMAWYRQAPGKQRDLVAV | |
| | ISTVGITKYADSVKGRFTISRDNAKNTVYLQMNSLRPEDTALYYCTSDARRG | |
| | WDTRYWGQGTLVTVSSA | |
| A041600030 | EVQLVESGGGSVQPGGSLRLSCAASGSIFSSTAMAWYRQAPGKRRDLVAAIN | 183 |
| (D1E) | TVGVTKYADSVKGRFTISRDNAKNTVYLQMNSLRPEDTALYYCTSDPRRGWD | |
| | TRYWGQGTLVTVSSGGGGSGGGGSGGGGSGGGGSEVQLVESGGGVVQPGGSL | |
| | RLSCAASGFTFRSFGMSWVRQAPGKGPEWVSSISGSGSDTLYADSVKGRFTI | |
| | SRDNSKNTLYLQMNSLRPEDTALYYCTIGGSLSRSSQGTLVTVSSGGGGSGG | |
| | GGSGGGGSGGGGSGGGGSGGGGSGGGGSEVQLVESGGGSVQPGGSLRLSCAA | |
| | SGSIFSSTAMAWYRQAPGKQRDLVAVISTVGITKYADSVKGRFTISRDNAKN | |
| | TVYLQMNSLRPEDTALYYCTSDARRGWDTRYWGQGTLVTVSSA | |
| A041600031 | EVQLVESGGGSVQPGGSLRLSCAASGSIFSSTAMAWYRQAPGKRRDLVAAIN | 184 |
| (D1E) | TVGVTKYADSVKGRFTISRDNAKNTVYLQMNSLRPEDTALYYCTSDPRRGWD | |
| | TRYWGQGTLVTVSSGGGGSGGGSEVQLVESGGGVVQPGGSLRLSCAASGFTF | |
| | RSFGMSWVRQAPGKGPEWVSSISGSGSDTLYADSVKGRFTISRDNSKNTLYL | |
| | QMNSLRPEDTALYYCTIGGSLSRSSQGTLVTVSSGGGGSGGGSEVQLVESGG | |
| | GSVQPGGSLRLSCAASGSIFSSTAMAWYRQAPGKQRDLVAVISTVGITKYAD | |
| | SVKGRFTISRDNAKNTVYLQMNSLRPEDTALYYCTSDARRGWDTRYWGQGTL | |
| | VTVSSA | |
| A041600032 | EVQLVESGGGSVQPGGSLRLSCAASGSIFSSTAMAWYRQAPGKRRDLVAAIN | 185 |
| (D1E) | TVGVTKYADSVKGRFTISRDNAKNTVYLQMNSLRPEDTALYYCTSDPRRGWD | |
| | TRYWGQGTLVTVSSGGGGSGGGGSGGGGSGGGGSGGGGGGGGSGGGGSEVQL | |
| | VESGGGVVQPGGSLRLSCAASGFTFRSFGMSWVRQAPGKGPEWVSSISGSGS | |
| | DTLYADSVKGRFTISRDNSKNTLYLQMNSLRPEDTALYYCTIGGSLSRSSQG | |
| | TLVTVSSGGGGSGGGSEVQLVESGGGSVQPGGSLRLSCAASGSIFSSTAMAW | |
| | YRQAPGKQRDLVAVISTVGITKYADSVKGRFTISRDNAKNTVYLQMNSLRPE | |
| | DTALYYCTSDARRGWDTRYWGQGTLVTVSSA | |
| A041600033 | EVQLVESGGGSVQPGGSLRLSCAASGSIFSSTAMAWYRQAPGKRRDLVAAIN | 186 |
| (D1E) | TVGVTKYADSVKGRFTISRDNAKNTVYLQMNSLRPEDTALYYCTSDPRRGWD | |
| | TRYWGQGTLVTVSSGGGGSGGGGSGGGGSGGGGSGGGGSGGGGSGGGGSEVQ | |
| | LVESGGGVVQPGGSLRLSCAASGFTFRSFGMSWVRQAPGKGPEWVSSISGSG | |
| | SDTLYADSVKGRFTISRDNSKNTLYLQMNSLRPEDTALYYCTIGGSLSRSSQ | |
| | GTLVTVSSGGGGSGGGGSGGGGSGGGGSEVQLVESGGGSVQPGGSLRLSCAA | |
| | SGSIFSSTAMAWYRQAPGKQRDLVAVISTVGITKYADSVKGRFTISRDNAKN | |
| | TVYLQMNSLRPEDTALYYCTSDARRGWDTRYWGQGTLVTVSSA | |
| A041600034 | EVQLVESGGGSVQPGGSLRLSCAASGSIFSSTAMAWYRQAPGKQRDLVAVIS | 187 |
| (D1E) | TVGITKYADSVKGRFTISRDNAKNTVYLQMNSLRPEDTALYYCTSDARRGWD | |
| | TRYWGQGTLVTVSSGGGGSGGGSEVQLVESGGGVVQPGGSLRLSCAASGFTE | |
| | RSFGMSWVRQAPGKGPEWVSSISGSGSDTLYADSVKGRFTISRDNSKNTLYL | |
| | QMNSLRPEDTALYYCTIGGSLSRSSQGTLVTVSSGGGGSGGGSEVQLVESGG | |
| | GSVQPGGSLRLSCAASGSIFSSTAMAWYRQAPGKQRDLVAVISTVGITKYAD | |
| | SVKGRFTISRDNAKNTVYLQMNSLRPEDTALYYCTSDARRGWDTRYWGQGTL | |
| | VTVSSA | |
| A041600035 | EVQLVESGGGSVQPGGSLRLSCAASGSIFSSTAMAWYRQAPGKQRDLVAVIS | 188 |
| (D1E) | TVGITKYADSVKGRFTISRDNAKNTVYLQMNSLRPEDTALYYCTSDARRGWD | |
| | TRYWGQGTLVTVSSGGGGSGGGSEVQLVESGGGSVQPGGSLRLSCAASGSIF | |
| | SSTAMAWYRQAPGKQRDLVAVISTVGITKYADSVKGRFTISRDNAKNTVYLQ | |
| | MNSLRPEDTALYYCTSDARRGWDTRYWGQGTLVTVSSGGGGSGGGSEVQLVE | |
| | SGGGVVQPGGSLRLSCAASGFTERSFGMSWVRQAPGKGPEWVSSISGSGSDT | |
| | LYADSVKGRFTISRDNSKNTLYLQMNSLRPEDTALYYCTIGGSLSRSSQGTL | |
| | VTVSSA | |
| A041600036 | EVQLVESGGGSVQPGGSLRLSCAASGSIFSSTAMAWYRQAPGKRRDLVAAIN | 189 |
| (D1E) | TVGVTKYADSVKGRFTISRDNAKNTVYLQMNSLRPEDTALYYCTSDPRRGWD | |
| | TRYWGQGTLVTVSSGGGGSGGGGSGGGGSGGGGSEVQLVESGGGSVQPGGSL | |
| | RLSCAASGSIFSSTAMAWYRQAPGKRRDLVAAINTVGVTKYADSVKGRFTIS | |
| | RDNAKNTVYLQMNSLRPEDTALYYCTSDPRRGWDTRYWGQGTLVTVSSGGGG | |
| | SGGGSEVQLVESGGGVVQPGGSLRLSCAASGFTFRSFGMSWVRQAPGKGPEW | |
| | VSSISGSGSDTLYADSVKGRFTISRDNSKNTLYLQMNSLRPEDTALYYCTIG | |
| | GSLSRSSQGTLVTVSSA | |
| A041600037 | EVQLVESGGGSVQPGGSLRLSCAASGSIFSSTAMAWYRQAPGKRRDLVAAIN | 190 |
| (D1E) | TVGVTKYADSVKGRFTISRDNAKNTVYLQMNSLRPEDTALYYCTSDPRRGWD | |
| | TRYWGQGTLVTVSSGGGGSGGGGSGGGGSGGGGSEVQLVESGGGSVQPGGSL | |
| | RLSCAASGSIFSSTAMAWYRQAPGKRRDLVAAINTVGVTKYADSVKGRFTIS | |
| | RDNAKNTVYLQMNSLRPEDTALYYCTSDPRRGWDTRYWGQGTLVTVSSGGGG | |
| | SGGGGSGGGGSGGGGSGGGGSGGGGSGGGGSEVQLVESGGGVVQPGGSLRLS | |
| | CAASGFTFRSFGMSWVRQAPGKGPEWVSSISGSGSDTLYADSVKGRFTISRD | |
| | NSKNTLYLQMNSLRPEDTALYYCTIGGSLSRSSQGTLVTVSSA | |
| A041600038 | EVQLVESGGGSVQPGGSLRLSCAASGSIFSSTAMAWYRQAPGKRRDLVAAIN | 191 |
| | TVGVTKYADSVKGRFTISRDNAKNTVYLQMNSLRPEDTALYYCTSDPRRGWD | |
| | TRYWGQGTLVTVSSGGGGSGGGGGGGGSGGGGSGGGGSGGGGSGGGGSEVQL | |
| | VESGGGSVQPGGSLRLSCAASGSIFSSTAMAWYRQAPGKRRDLVAAINTVGV | |
| | TKYADSVKGRFTISRDNAKNTVYLQMNSLRPEDTALYYCTSDPRRGWDTRYW | |
| | GQGTLVTVSSGGGGSGGGSEVQLVESGGGVVQPGGSLRLSCAASGFTFRSFG | |
| | MSWVRQAPGKGPEWVSSISGSGSDTLYADSVKGRFTISRDNSKNTLYLQMNS | |
| | LRPEDTALYYCTIGGSLSRSSQGTLVTVSSA | |
| A041600039 | EVQLVESGGGSVQPGGSLRLSCAASGSIFSSTAMAWYRQAPGKRRDLVAAIN | 192 |
| (D1E) | TVGVTKYADSVKGRFTISRDNAKNTVYLQMNSLRPEDTALYYCTSDPRRGWD | |
| | TRYWGQGTLVTVSSGGGGSGGGGSGGGGSGGGGSGGGGGGGGSGGGGSEVQL | |
| | VESGGGSVQPGGSLRLSCAASGSIFSSTAMAWYRQAPGKRRDLVAAINTVGV | |
| | TKYADSVKGRFTISRDNAKNTVYLQMNSLRPEDTALYYCTSDPRRGWDTRYW | |
| | GQGTLVTVSSGGGGSGGGGSGGGGSGGGGSEVQLVESGGGVVQPGGSLRLSC | |
| | AASGFTFRSFGMSWVRQAPGKGPEWVSSISGSGSDTLYADSVKGRFTISRDN | |
| | SKNTLYLQMNSLRPEDTALYYCTIGGSLSRSSQGTLVTVSSA | |
| A041600040 | EVQLVESGGGSVQPGGSLRLSCAASGSIFSSTAMAWYRQAPGKRRDLVAAIN | 193 |
| (D1E) | TVGVTKYADSVKGRFTISRDNAKNTVYLQMNSLRPEDTALYYCTSDPRRGWD | |
| | TRYWGQGTLVTVSSGGGGSGGGSEVQLVESGGGSVQPGGSLRLSCAASGSIF | |
| | SSTAMAWYRQAPGKQRDLVAVISTVGITKYADSVKGRFTISRDNAKNTVYLQ | |
| | MNSLRPEDTALYYCTSDARRGWDTRYWGQGTLVTVSSGGGGSGGGSEVQLVE | |
| | SGGGVVQPGGSLRLSCAASGFTFRSFGMSWVRQAPGKGPEWVSSISGSGSDT | |
| | LYADSVKGRFTISRDNSKNTLYLQMNSLRPEDTALYYCTIGGSLSRSSQGTL | |
| | VTVSSA | |
| A041600041 | EVQLVESGGGSVQPGGSLRLSCAASGSIFSSTAMAWYRQAPGKQRDLVAVIS | 194 |
| (D1E) | TVGITKYADSVKGRFTISRDNAKNTVYLQMNSLRPEDTALYYCTSDARRGWD | |
| | TRYWGQGTLVTVSSGGGGSGGGSEVQLVESGGGSVQPGGSLRLSCAASGSIF | |
| | SSTAMAWYRQAPGKRRDLVAAINTVGVTKYADSVKGRFTISRDNAKNTVYLQ | |
| | MNSLRPEDTALYYCTSDPRRGWDTRYWGQGTLVTVSSGGGGSGGGSEVQLVE | |
| | SGGGVVQPGGSLRLSCAASGFTFRSFGMSWVRQAPGKGPEWVSSISGSGSDT | |
| | LYADSVKGRFTISRDNSKNTLYLQMNSLRPEDTALYYCTIGGSLSRSSQGTL | |
| | VTVSSA | |
| A041600042 | EVQLVESGGGSVQPGGSLRLSCAASGSIFSSTAMAWYRQAPGKRRDLVAAIN | 195 |
| (D1E) | TVGVTKYADSVKGRFTISRDNAKNTVYLQMNSLRPEDTALYYCTSDPRRGWD | |
| | TRYWGQGTLVTVSSGGGGSGGGGSGGGGSGGGGSEVQLVESGGGSVQPGGSL | |
| | RLSCAASGSIFSSTAMAWYRQAPGKRRDLVAAINTVGVTKYADSVKGRFTIS | |
| | RDNAKNTVYLQMNSLRPEDTALYYCTSDPRRGWDTRYWGQGTLVTVSSGGGG | |
| | SGGGGSGGGGSGGGGSEVQLVESGGGVVQPGGSLRLSCAASGFTFRSFGMSW | |
| | VRQAPGKGPEWVSSISGSGSDTLYADSVKGRFTISRDNSKNTLYLQMNSLRP | |
| | EDTALYYCTIGGSLSRSSQGTLVTVSSA | |
| A041600043 | EVQLVESGGGSVQPGGSLRLSCAASGSIFSSTAMAWYRQAPGKRRDLVAAIN | 196 |
| (D1E) | TVGVTKYADSVKGRFTISRDNAKNTVYLQMNSLRPEDTALYYCTSDPRRGWD | |
| | TRYWGQGTLVTVSSGGGGSGGGGSGGGGSGGGGSGGGGSGGGGSGGGGSEVQ | |
| | LVESGGGSVQPGGSLRLSCAASGSIFSSTAMAWYRQAPGKRRDLVAAINTVG | |
| | VTKYADSVKGRFTISRDNAKNTVYLQMNSLRPEDTALYYCTSDPRRGWDTRY | |
| | WGQGTLVTVSSGGGGGGGGSGGGGSGGGGSGGGGSGGGGSGGGGSEVQLVES | |
| | GGGVVQPGGSLRLSCAASGFTFRSFGMSWVRQAPGKGPEWVSSISGSGSDTL | |
| | YADSVKGRFTISRDNSKNTLYLQMNSLRPEDTALYYCTIGGSLSRSSQGTLV | |
| | TVSSA | |
| A041600044 | EVQLVESGGGSVQPGGSLRLSCAASGSIFSSTAMAWYRQAPGKRRDLVAAIN | 197 |
| (D1E) | TVGVTKYADSVKGRFTISRDNAKNTVYLQMNSLRPEDTALYYCTSDPRRGWD | |
| | TRYWGQGTLVTVSSGGGGSGGGSEVQLVESGGGSVQPGGSLRLSCAASGSIF | |
| | SSTAMAWYRQAPGKRRDLVAAINTVGVTKYADSVKGRFTISRDNAKNTVYLQ | |
| | MNSLRPEDTALYYCTSDPRRGWDTRYWGQGTLVTVSSGGGGSGGGGSGGGGS | |
| | GGGGSEVQLVESGGGVVQPGGSLRLSCAASGFTFRSFGMSWVRQAPGKGPEW | |
| | VSSISGSGSDTLYADSVKGRFTISRDNSKNTLYLQMNSLRPEDTALYYCTIG | |
| | GSLSRSSQGTLVTVSSA | |
| A041600045 | EVQLVESGGGSVQPGGSLRLSCAASGSIFSSTAMAWYRQAPGKRRDLVAAIN | 198 |
| (D1E) | TVGVTKYADSVKGRFTISRDNAKNTVYLQMNSLRPEDTALYYCTSDPRRGWD | |
| | TRYWGQGTLVTVSSGGGGSGGGSEVQLVESGGGSVQPGGSLRLSCAASGSIF | |
| | SSTAMAWYRQAPGKRRDLVAAINTVGVTKYADSVKGRFTISRDNAKNTVYLQ | |
| | MNSLRPEDTALYYCTSDPRRGWDTRYWGQGTLVTVSSGGGGSGGGGSGGGGS | |
| | GGGGSGGGGSGGGGSGGGGSEVQLVESGGGVVQPGGSLRLSCAASGFTFRSF | |
| | GMSWVRQAPGKGPEWVSSISGSGSDTLYADSVKGRFTISRDNSKNTLYLQMN | |
| | SLRPEDTALYYCTIGGSLSRSSQGTLVTVSSA | |
| A041600046 | EVQLVESGGGSVQPGGSLRLSCAASGSIFSSTAMAWYRQAPGKRRDLVAAIN | 199 |
| (D1E) | TVGVTKYADSVKGRFTISRDNAKNTVYLQMNSLRPEDTALYYCTSDPRRGWD | |
| | TRYWGQGTLVTVSSGGGGSGGGGSGGGGSGGGGSGGGGSGGGGSGGGGSEVQ | |
| | LVESGGGVVQPGGSLRLSCAASGFTFRSFGMSWVRQAPGKGPEWVSSISGSG | |
| | SDTLYADSVKGRFTISRDNSKNTLYLQMNSLRPEDTALYYCTIGGSLSRSSQ | |
| | GTLVTVSSGGGGSGGGGSGGGGSGGGGSGGGGSGGGGSGGGGSEVQLVESGG | |
| | GSVQPGGSLRLSCAASGSIFSSTAMAWYRQAPGKQRDLVAVISTVGITKYAD | |
| | SVKGRFTISRDNAKNTVYLQMNSLRPEDTALYYCTSDARRGWDTRYWGQGTL | |
| | VTVSSA | |
| A041600085 | EVQLVESGGGLVQPGGSLRLSCAASGSIFSSTAMAWYRQAPGKRRDLVAAIS | 200 |
| (D1E) | TVGVTKYADSVKGRFTISRDNSKNTVYLQMNSLRPEDTAVYYCTSDPRRGWD | |
| | TRYWGQGTLVTVSSGGGGSGGGSEVQLVESGGGVVQPGGSLRLSCAASGFTF | |
| | RSFGMSWVRQAPGKGPEWVSSISGSGSDTLYADSVKGRFTISRDNSKNTLYL | |
| | QMNSLRPEDTALYYCTIGGSLSRSSQGTLVTVSSGGGGSGGGSEVQLVESGG | |
| | GLVQPGGSLRLSCAASGSIFSSTAMAWYRQAPGKRRDLVAAISTVGVTKYAD | |
| | SVKGRFTISRDNSKNTVYLQMNSLRPEDTAVYYCTSDPRRGWDTRYWGQGTL | |
| | VTVSSA | |
| A041600087 | EVQLVESGGGLVQPGGSLRLSCAASGSIFSSTAMAWYRQAPGKRRDLVAAIS | 201 |
| (D1E) | TVGVTKYADSVKGRFTISRDNSKNTVYLQMNSLRPEDTAVYYCTSDPRRGWD | |
| | TRYWGQGTLVTVSSGGGGSGGGSEVQLVESGGGLVQPGNSLRLSCAASGFTF | |
| | SSFGMSWVRQAPGKGLEWVSSISGSGSDTLYADSVKGRFTISRDNAKTTLYL | |
| | QMNSLRPEDTAVYYCTIGGSLSRSSQGTLVTVSSGGGGSGGGSEVQLVESGG | |
| | GLVQPGGSLRLSCAASGSIFSSTAMAWYRQAPGKRRDLVAAISTVGVTKYAD | |
| | SVKGRFTISRDNSKNTVYLQMNSLRPEDTAVYYCTSDPRRGWDTRYWGQGTL | |
| | VTVSS | |

TABLE A-9
CX3CR1 sequences.

| Name [further | | |
|---|---|---|
| info] | Sequence | ID |
| human CX3CR1 | MDQFPESVTENFEYDDLAEACYIGDIVVFGTVFLSIFYSVIFAIGLVGNLLVV | 84 |
| (homo sapiens) | FALTNSKKPKSVTDIYLLNLALSDLLFVATLPFWTHYLINEKGLHNAMCKETT | |
| | AFFFIGFFGSIFFITVISIDRYLAIVLAANSMNNRTVQHGVTISLGVWAAAIL | |
| | VAAPQFMFTKQKENECLGDYPEVLQEIWPVLRNVETNFLGFLLPLLIMSYCYF | |
| | RIIQTLFSCKNHKKAKAIKLILLVVIVFFLFWTPYNVMIFLETLKLYDFFPSC | |
| | DMRKDLRLALSVTETVAFSHCCLNPLIYAFAGEKFRRYLYHLYGKCLAVLCGR | |
| | SVHVDFSSSESQRSRHGSVLSSNFTYHTSDGDALLLL | |
| cynomolgus | MDPFPESVTENFEYDDSAEACYIGDIVAFGTVFLSIFYSVVFAIGLVGNLLVV | 85 |
| CX3CR1 (Macaca | FALTNSKKPKSVTDIYLLNLALSDLLFVATLPFWTHYVINEEGLQNAMCKETT | |
| fascicularis) | AFFFIGFFGSIFFITIISIDRYLAIVLAANSMNNRTVQHGVTISLGVWAAAIL | |
| | VAAPQFMFTKQKENECLGDYPEVLQEIWPVLRNVEANFLGFLLPLLIMSYCYF | |
| | RIIQTLFSCKNHKKAKAIKLILLVVVVFFLFWTPYNVMIFLETLKLYDFFPSC | |
| | DMRRDLRLALSVTETVAFSHCCLNPLIYAFAGEKERRYLYHLYGKCLAVLCGR | |
| | SVHVDFSPSESQRSRQGSVLSSNFTYHTSDGDASLLL | |

TABLE A-10
Additional sequences.

| Name [further info] | Sequence | ID |
|---|---|---|
| C-terminus of FR4 | VTVSS | 135 |

TABLE A-11
Combinations of hallmark residues in
an ISVD polypeptide (see Table 2).

| Name [further info] | Sequence | ID |
|---|---|---|
| ISVD, positions 43-46 | KERE | 86 |
| [notes (1), (3), (4)] | | |
| ISVD, positions 43-46 | KORE | 87 |
| [notes (1), (3), (4)] | | |
| ISVD, positions 44-47 | GLEW | 88 |
| [notes (2), (4), (6), | | |
| (7)] | | |
| ISVD, positions 43-46 | KEREL | 89 |
| [note (3)] | | |
| | KEREF | 90 |
| | KOREL | 91 |
| | KQREF | 92 |
| | KEREG | 93 |
| | KOREW | 94 |
| | KQREG | 95 |
| | TERE | 96 |
| | TEREL | 97 |
| | TQRE | 98 |
| | TQREL | 99 |
| | KECE | 100 |
| | KECEL | 101 |
| | KECER | 102 |
| | KQCE | 103 |
| | KQCEL | 104 |
| | RERE | 105 |
| | REREG | 106 |
| | RQRE | 107 |
| | RQREL | 108 |
| | RQREF | 109 |
| | RQREW | 110 |
| | QERE | 111 |
| | QEREG | 112 |
| | QQRE | 113 |
| | QQREW | 114 |
| | QQREL | 115 |
| | QQREF | 116 |
| | KGRE | 117 |
| | KGREG | 118 |
| | KDRE | 119 |
| | KDREV | 120 |
| | DECKL | 121 |
| | NVCEL | 122 |
| ISVD, positions 44-47 | GVEW | 123 |
| [note (8)] | | |
| | EPEW | 124 |
| | GLER | 125 |
| | DQEW | 126 |
| | DLEW | 127 |
| | GIEW | 128 |
| | ELEW | 129 |
| | GPEW | 130 |
| | EWLP | 131 |
| | GPER | 132 |
| | GLER | 133 |
| | ELEW | 134 |

### 9. LIST OF REFERENCES

Imai et al., Cell 1997 Nov. 14; 91(4):521-30, PMID: 9390561
Umehara et al., Arterioscler Thromb Vasc Biol. 2004 January; 24(1):34-40; PMID: 12969992
Faure et al., Science 2000 Mar. 24; 287(5461):2274-7; PMID: 10731151
McDermott et al. Circ Res. 2001 Aug. 31; 89(5):401-7, PMID: 11532900
Moatti et al. Blood 2001 Apr. 1; 97(7):1925-8, PMID: 11264153
McDermott, 2003; J. Clin. Invest. 111:1241, PMID: 12697743
Ghilardi, 2004, Stroke 35:1276, PMID: 15118174
Combadiere, 2003; Circulation, 107:1009, PMID: 12600915
Lesnik, 2003; J. Clin. Invest. 111:333, PMID: 12569158
Wakita et al. 2017, Mol. Pharmacol. 92: 502, PMID: 28842393
Tanaka et al. 2021, Modern Rheumatol 31783, PMID: 33427546
WO 2013/130381
Low et al. 2020, Mabs, 12(1):1709322, PMID: 31924119
Hamers-Casterman et al. Nature 363: 446-448, 1993
review article by Muyldermans (Reviews in Molecular Biotechnology 74: 277-302, 2001)
WO 94/04678
Ward et al (see for example WO 94/04678 and Riechmann, Febs Lett., 339:285-290, 1994 and Prot. Eng., 9:531-537, 1996)
Conrath et al., J. Biol. Chem., Vol. 276, 10. 7346-7350, 2001, as well as to for example WO 96/34103 and WO 99/23221
WO 2008/020079
Davies and Riechman (1994 and 1996)
Kabat et al. (“Sequence of proteins of immunological interest”, US Public Health Services, NIH Bethesda, MD, Publication No. 91)
Riechmann and Muyldermans, 2000 (J. Immunol. Methods 240 (1-2): 185-195
Kontermann and Dubel (Eds. 2010, Antibody Engineering, vol 2, Springer Verlag Heidelberg Berlin, Martin, Chapter 3, pp. 33-51)
WO 94/25591, Gasser et al. Biotechnol. Bioeng. 94: 535, 2006; Gasser et al. Appl. Environ. Microbiol. 73: 6499, 2007; or Damasceno et al. Microbiol. Biotechnol. 74: 381, 2007
Estep P. et al. 2013 MAbs. 5(2): 270-8. PMID: 23575269

1. A polypeptide comprising or consisting of at least three immunoglobulin single variable domains (ISVDs), wherein each of the at least three ISVDs has the structure FR1-CDR1-FR2-CDR2-FR3-CDR3-FR4 in which FR1 to FR4 refer to framework regions and CDR1 to CDR3 refer to complementarity determining regions; and wherein the polypeptide comprises;
(a) a first ISVD comprising (according to AbM numbering):
a CDR1 that is the amino acid sequence of SEQ ID NO: 10;
a CDR2 that is the amino acid sequence of SEQ ID NO: 12; and
a CDR3 that is the amino acid sequence of SEQ ID NO: 14; and
(b) a second ISVD comprising (according to AbM numbering):
a CDR1 that is the amino acid sequence of SEQ ID NO: 10;
a CDR2 that is the amino acid sequence of SEQ ID NO: 12; and
a CDR3 that is the amino acid sequence of SEQ ID NO: 14; and
(c) a third ISVD comprising (according to AbM numbering):
a CDR1 that is the amino acid sequence of SEQ ID NO: 53 or an amino acid sequence with 2 or 1 amino acid difference(s) with SEQ ID NO: 53;
a CDR2 that is the amino acid sequence of SEQ ID NO: 55 or an amino acid sequence with 2 or 1 amino acid difference(s) with SEQ ID NO: 55; and
a CDR3 that is the amino acid sequence of SEQ ID NO: 57 or an amino acid sequence with 2 or 1 amino acid difference(s) with SEQ ID NO: 57; and
wherein (a), (b), and (c) are linked directly or via a peptidic linker.

2. The polypeptide according to claim 1, wherein:
the ISVD in (a), (b), or (c) is a C-terminal ISVD of the polypeptide, and wherein the C-terminal ISVD carries a C-terminal extension of a single alanine (A) residue;
the order of (a), (b), and (c) indicates their relative position to each other within the polypeptide, considered from the N-terminus to the C-terminus of said polypeptide; or
a combination thereof.

3. (canceled)

4. The polypeptide according to claim 1, wherein in (a), in (b) and in (c) the ISVD is a VH domain or a VHH domain, optionally wherein in (a), in (b) and in (c) the ISVD is a camelized VH domain, a humanized VHH domain, a domain antibody, or a dAb.

5. (canceled)

6. The polypeptide according to claim 1, wherein in (a) and in (b), the ISVD comprises one or more selected from an aspartic acid (D) at amino acid position 1 according to Kabat numbering, a serine (S) at amino acid position 11 according to Kabat numbering, a proline (P) at amino acid position 14 according to Kabat numbering, a glycine (G) at amino acid position 16 according to Kabat numbering, a threonine (T) at amino acid position 32 according to Kabat numbering, a glutamine (Q) at position 44 according to Kabat numbering, a valine (V) at amino acid position 50 according to Kabat numbering, a serine (S) at amino acid position 52 according to Kabat numbering, a threonine (T) at amino acid position 53 according to Kabat numbering, an isoleucine (I) at amino acid position 56 according to Kabat numbering, an arginine (R) at amino acid position 83 according to Kabat numbering, a leucine (L) at amino acid position 89 according to Kabat numbering, an alanine (A) at amino acid position 96 according to Kabat numbering, and a leucine (L) at amino acid position 108 according to Kabat numbering.

7.-9. (canceled)

10. The polypeptide according to claim 1, wherein in (a) the ISVD consists of an amino acid sequence as shown in SEQ ID NO: 3 or an amino acid that has at least 90% sequence identity with SEQ ID NO:3 and in (b) the ISVD consists of an amino acid sequence as shown in SEQ ID NO: 2 or an amino acid that has at least 90% sequence identity with SEQ ID NO: 2, wherein for the purpose of determining the % sequence identity, the amino acid sequence of the CDR regions are disregarded, optionally wherein the order of (a), (b), and (c) indicates their relative position to each other within the polypeptide, considered from the N-terminus to the C-terminus of said polypeptide.

11. The polypeptide according claim 1, wherein in (a) the ISVD consists of an amino acid sequence as shown in SEQ ID NO: 3 or an amino acid sequence having two or one amino acid difference(s) with SEQ ID NO: 2 and in (b) the ISVD consists of an amino acid sequence as shown in SEQ ID NO: 2 or an amino acid sequence having two or one amino acid difference(s) with SEQ ID NO: 2, optionally wherein the order of (a), (b), and (c) indicates their relative position to each other within the polypeptide, considered from the N-terminus to the C-terminus of said polypeptide.

12.-13. (canceled)

14. The polypeptide according to claim 1, wherein in (c) the ISVD binds to human serum albumin, optionally wherein in (c):
the ISVD comprises (according to AbM numbering)
a CDR1 that is the amino acid sequence of SEQ ID NO: 53;
a CDR2 that is the amino acid sequence of SEQ ID NO: 55; and
a CDR3 that is the amino acid sequence of SEQ ID NO: 57;
the ISVD comprises or consists of an amino acid sequence as shown in SEQ ID NO: 37 or an amino acid sequence having a sequence identity of more than 90% identity or more than 95% identity with SEQ ID NO: 37; or
a combination thereof.

15.-17. (canceled)

18. The polypeptide according to claim 1, wherein in (a) and in (b) the ISVD binds to fractalkine receptor CX3CR1 and consists of an amino acid sequence as shown in SEQ ID NO: 2 or an amino acid sequence having two or one amino acid difference(s) with SEQ ID NO: 2 and in (c) the ISVD binds to albumin and consists of an amino acid sequence as shown in SEQ ID NO: 37.

19. (canceled)

20. The polypeptide according to claim 1, wherein the polypeptide comprises two peptidic linkers, wherein each peptidic linker is located between two ISVDs within the polypeptide; optionally wherein the two peptidic linkers comprise or consist of an amino acid sequence as shown in SEQ ID NO: 69.

21. The polypeptide according to claim 1, wherein the polypeptide comprises an amino acid sequence having a sequence identity of more than 90% identity with SEQ ID NO: 82, optionally wherein the polypeptide comprises or consists of an amino acid sequence as shown in SEQ ID NO: 82.

22. The polypeptide according to claim 1, wherein the polypeptide comprises or consists of an amino acid sequence as shown in SEQ ID NO: 82.

23. The polypeptide according to claim 1, wherein the first ISVD and/or the second ISVD binds to a single nucleotide polymorphism (SNP) variant of CX3CR1, wherein the single nucleotide polymorphism (SNP) variant of CX3CR1 comprises at least one SNP selected from the group consisting of SNPs rs938203, rs2669849, rs1050592, rs3732379, and rs3732378.

24. The polypeptide according to claim 1, wherein the polypeptide:
is characterized by an increased yield after expression in yeast Pichia pastoris as compared to a reference polypeptide consisting of an amino acid sequence as shown in SEQ ID NO: 83;
is characterized by at least one improved biophysical property as compared to a reference polypeptide consisting of an amino acid sequence as shown in SEQ ID NO: 83;
is characterized by a higher recovery after a purification step as compared to the recovery of a reference polypeptide consisting of an amino acid sequence as shown in SEQ ID NO: 83;
has a reduced amount of missing disulfide bridges after expression of the polypeptide in a suitable host compared to a reference polypeptide consisting of an amino acid sequence as shown in SEQ ID NO: 83;
binds cell surface glycoprotein MUC18 with a lower affinity compared to the binding by a reference polypeptide consisting of an amino acid sequence as shown in SEQ ID NO: 83;
or a combination thereof.

25.-28. (canceled)

29. A polynucleotide encoding a polypeptide according to claim 1.

30. A host or host cell comprising a polynucleotide according to claim 1.

31. A composition comprising a polypeptide according to claim 1 or a polynucleotide encoding the same; optionally wherein the composition is a pharmaceutical composition; and further optionally wherein the composition comprises at least one pharmaceutically acceptable carrier, diluent, excipient and/or adjuvant.

32. A method for producing a polypeptide according to claim 1 comprising the step of:
expressing the polypeptide in a suitable host cell or host organism or in another suitable expression system;
and further optionally
isolating and/or purifying the polypeptide.

33.-34. (canceled)

35. A method of diagnosing a disease, disorder, or condition comprising contacting a sample that has been obtained from a subject with a polypeptide according to claim 1; optionally wherein the disease, disorder, or condition is associated with a fractalkine receptor CX3CR1 or a single nucleotide polymorphism (SNP) variant of CX3CR1.

36. (canceled)

37. A method for inhibiting the binding of CX3CR1 to fractalkine in a mammalian cell, comprising administering to the cell a polypeptide according to claim 1, whereby signaling mediated by the fractalkine is inhibited.

38. A method of treating a disease or disorder, wherein the disease or disorder is associated with a fractalkine receptor CX3CR1 or a single nucleotide polymorphism (SNP) variant of CX3CR1, optionally wherein the disease or disorder is selected from inflammatory disease, cardio- and cerebrovascular atherosclerotic disorder, peripheral artery disease, myocardial infarction, restenosis, diabetic nephropathy, glomerulonephritis, human crescentic glomerulonephritis, IgA nephropathy, membranous nephropathy, lupus nephritis, vasculitis including Henoch-Schonlein purpura and Wegener's granulomatosis, rheumatoid arthritis, graft versus host disease, atopic dermatitis, inflammatory bowel disease, Crohn's disease, osteoarthritis, allograft rejection, systemic sclerosis, neurodegenerative disorder and demyelinating disease, multiple sclerosis (MS), Alzheimer's disease, pulmonary disease such as COPD, asthma, neuroinflammation, neuropathic pain, inflammatory pain, and cancer, including ovarian cancer, and wherein said method comprises administering, to a subject in need thereof, a pharmaceutically active amount of the polypeptide according to claim 1.
